SEQUENCE LISTING (16 sequences)

SEQ ID NO: 1 - protein, 541 aa, Artificial sequence
  CLTX NK-CAR protein
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 422 aa, Artificial sequence
  CLTX-T-CAR protein
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 498 aa, Artificial sequence
  IL-13 T-CAR protein
MLLLVTSLLLCELPHPAFLLIPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 25 bp, Artificial sequenece
  Positive genotyping primer1
ctgtttccccttcccaggcaggtcc

SEQ ID NO: 5 - DNA, 25 bp, Artificial sequence
  Positive genotyping primer2
tcgtcgcgggtggcgaggcgcaccg

SEQ ID NO: 6 - DNA, 20 bp, Artificial sequence
  Homozygous screening primer1
cggttaatgtggctctggtt

SEQ ID NO: 7 - DNA, 20 bp, Artificial sequence
  Homozygous screening primer2
gagagagatggctccaggaa

SEQ ID NO: 8 - protein, 387 aa, Artificial sequence
  CLTX CD32 Fc-garma-R-CAR amino acid sequence
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 9 - protein, 497 aa, Artificial sequence
  anti-PSMA nanobody T-CAR amino acid sequence
MLLLVTSLLLCELPHPAFLLIPEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 626 aa, Artificial sequence
  anti-PSMA J-591-scFV T-CAR amino acid sequence
MLLLVTSLLLCELPHPAFLLIPEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGSTSGGGSGGGSGGGGSSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFRVKFSRSADAPAYQQGQNQQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1626 bp, Artificial sequence
  DNA for protein SEQ ID NO 1
atgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaccattttttttctgctgcttcatcgctgtagccatgggaatccgtttcattattatggtaacatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 12 - DNA, 1269 bp, Artificial sequence
  DNA for protein SEQ ID NO 2
atgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatggccctgattgtgctggggggcgtcgccggcctcctgcttttcattgggctaggcatcttcttcagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 13 - DNA, 1497 bp, Artificial sequence
  DNA for protein SEQ ID NO 3
atgcttctcctggtgacaagccttctgctctgtgagttaccacacccagcattcctcctgatcccaggccctgtgcctccctctacagccctcaggtacctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatggccctgattgtgctggggggcgtcgccggcctcctgcttttcattgggctaggcatcttcttcagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 14 - DNA, 1164 bp, Artificial sequence
  DNA for protein SEQ ID NO 8
atgcttctcctggtgacaagccttctgctctgtgagttaccacacccagcattcctcctgatcccaatgtgcatgccttgcttcaccaccgaccaccagatggccagaaagtgcgatgactgctgcggcggcaagggcagaggcaagtgctacggcccccagtgtctgtgcagagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 15 - DNA, 1494 bp, Artificial sequence
  DNA for protein SEQ ID NO 9
atgcttctcctggtgacaagccttctgctctgtgagttaccacacccagcattcctcctgatcccagaagtgcagcttgtagagagcggcgggggcctagttcaacccggcggcagcctgaccctgagctgcgccgctagcagattcatgatcagcgagtacagcatgcactgggtgagacaagcccccggcaagggcctggagtgggtgagcaccatcaaccccgccggcaccaccgactacgccgagagcgtgaagggcagattcaccatcagcagagacaacgccaagaacaccctgtacctgcagatgaacagcctgaagcccgaggacaccgccgtgtactactgcgacggctacggctacagaggccaaggcacccaagtgaccgtgagcagcgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatggccctgattgtgctggggggcgtcgccggcctcctgcttttcattgggctaggcatcttcttcagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 16 - DNA, 1881 bp, Artificial sequence
  DNA for protein SEQ ID NO 10
atgcttctcctggtgacaagccttctgctctgtgagttaccacacccagcattcctcctgatcccagaagtgcagctggtgcagtctggcgccgaagtgaagaaacctggcgcctccgtgaagatctcctgcaagacctccggctacaccttcaccgagtacaccatccactgggtgaaacaggcctccggcaagggcctggaatggatcggcaacatcaaccctaacaacggcggcaccacctacaaccagaagttcgaggaccgggccaccctgaccgtggacaagtccacctccaccgcctacatggaactgtcctccctgcggtctgaggacaccgccgtgtactactgcgccgctggctggaacttcgactactggggccagggcaccacagtgacagtctcgagcggctctacctctggcggaggctctgggggaggaagcggcggaggcggctcctctgacatcgtgatgacccagtccccctcctccctgtctgcctccgtgggcgacagagtgaccatcacatgcaaggcctcccaggatgtgggcaccgccgtggactggtatcagcagaagcctggcaaggcccctaagctgctgatctactgggcctccaccagacacaccggcgtgcctgacagattcaccggctccggctctggcaccgacttcaccctgaccatctccagcctgcagcctgaggacttcgccgactacttctgccagcagtacaactcctaccctctgaccttcggcggaggcaccaagctggaaatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatggccctgattgtgctggggggcgtcgccggcctcctgcttttcattgggctaggcatcttcttcagagtgaagttcagcaggagcgcagacgcccccgcgtaccagcagggccagaaccagcagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

(19) United States
(12) Patent Application Publication
(10) Pub. No.: US 2024/0024473 A1
(43) Pub. Date: Jan. 25, 2024

(54) HUMAN CHIMERIC ANTIGEN RECEPTOR NEUTROPHILS, COMPOSITIONS, KITS AND METHODS OF USE

(71) Applicant: Purdue Research Foundation, West Lafayette, IN (US)

(72) Inventors: Xiaoping BAO, West Lafayette, IN (US); Qing DENG, West Lafayette, IN (US); Yun CHANG, West Lafayette, IN (US); Ramizah MOHD SABRI, West Lafayette, IN (US)

(21) Appl. No.: 18/256,916

(22) PCT Filed: Dec. 10, 2021
(86) PCT No.: PCT/US2021/062734

§ 371 Date: Jun. 9, 2023

Related U.S. Application Data
(60) Provisional application No. 63/124,125, filed on Dec. 11, 2020.

(51) Int. Cl. A61K 39/00; C12N 5/0787; C12N 15/85; A61P 35/00; C07K 14/435

(52) U.S. Cl. CPC A61K 39/461; C12N 5/0642; C12N 15/85; A61K 39/4631; A61P 35/00; C07K 14/43522; C12N 2800/107; C12N 2506/03; C12N 2501/22; C12N 2501/15; C12N 2501/165; C12N 2501/125; C12N 2501/26; C12N 2501/385; C12N 2501/2303; C12N 2501/2306

(57) ABSTRACT

The present disclosure relates to a stage-specific process for manufacturing a population of neutrophils, such as chimeric antigen receptor-expressing (CAR-expressing) neutrophils (e.g., T cells and natural killer (NK) cells), from human pluripotent stem cells (hPSCs) using defined media and related compositions, kits, and methods of use (e.g., targeted cancer immunotherapy). Stage-specific processes for generating neutrophils and chimeric antigen receptor (CAR) neutrophils from human pluripotent stem cells (hPSCs) using chemically defined, feeder-free platforms and stage-specific morphogens; cell lines; pharmaceutical compositions; a method of treating cancer; and a kit are within the scopes of this disclosure.

### CROSS REFERENCE TO RELATED APPLICATION

This application claims priority to U.S. provisional patent application no. 63/124,125 filed Dec. 11, 2020. The contents of the provisional patent application and the appendix are hereby incorporated by reference in their entireties.

### STATEMENT OF SEQUENCE LISTING

A computer-readable form (CRF) of the Sequence Listing is submitted concurrently with this application. The file, 69243-02_Seq_Listing_ST25_txt, is generated on Dec. 6, 2021, file size: 39 kilobytes, which is herein incorporated by reference in its entirety. Applicant states that the content of the computer-readable form is the same and the information recorded in computer readable form is identical to the written sequence listing.

### TECHNICAL FIELD

The disclosure relates to a stage-specific process for manufacturing a population of neutrophils, such as chimeric antigen receptor-expressing (CAR-expressing) neutrophils (e.g., T cells and natural killer (NK) cells), from human pluripotent stem cells (hPSCs) using defined media and related compositions, kits, and methods of use (e.g., targeted cancer immunotherapy).

### BACKGROUND

This section introduces aspects that may help facilitate a better understanding of the disclosure. Accordingly, these statements are to be read in this light and are not to be understood as admissions about what is or is not prior art.

Neutrophils, the most abundant circulating leukocytes in humans, accumulate in many types of tumors and represent a significant portion of tumor-infiltrating cells (Eruslanov et al., 2017; Ilie et al., 2012; Jaillon et al., 2020; Zhao et al., 2020). Due to their heterogeneity and plasticity in the tumor microenvironment (TME), neutrophils have demonstrated contradictory pro-tumor and anti-tumor effects during tumor evolution. For instance, tumor-associated neutrophils (TANs) present direct or antibody-dependent cytotoxicity against solid cancers (Kargl et al., 2019; Matlung et al., 2018), whereas they also facilitate angiogenesis, promote tumor cell migration, and suppress the anti-tumor function of other immune cells in the TME (Coffelt et al., 2015, 2016; Huo et al., 2019). The presence of pro-tumor neutrophils has limited the efficacy of many cancer therapies (Itatani et al., 2020), including emerging immunotherapies, leading to the development of suppressive neutrophil-targeted strategies for treating various cancers in both preclinical studies and clinical trials (Zhao et al., 2020). However, given the high heterogeneity of neutrophils in the complex TME (Lecot et al., 2019; Sagiv et al., 2015), general suppression with small molecule- or antibody-based approaches may eliminate both pro-tumor and anti-tumor neutrophils, and reduce the efficacy of neutrophil-targeted therapy. Furthermore, adverse effects, such as neutropenia (McDermott et al., 2010), may develop in cancer patients and increase their risk of infections (Xie et al., 2020). Thus, alternative neutrophil-targeting approaches are needed to realize their full potential in cancer treatment.

For the past decade, chimeric antigen receptors (CARs) have been developed and widely used in T and natural killer (NK) cells to boost their anti-tumor effects on various hematologic malignancies and solid tumors (Feins et al., 2019; June and Sadelain, 2018; June et al., 2018; Mehta and Rezvani, 2018; Zhu et al., 2018), revolutionizing the field of cancer immunotherapy. More recently, genetic engineering of primary macrophages with CARs has programed and maintained them as anti-tumor effector cells both in vitro and in vivo (Klichinsky et al., 2020). Given their similarity to macrophages and shared innate anti-tumor response (Yan et al., 2014), we hypothesized that human neutrophils may present enhanced tumoricidal activities in various ways after CAR engineering. Such an approach has not yet been applied to neutrophils, largely due to their short half-life and difficulty in genome editing. To provide a solution to these unmet medical needs, we are encouraged to search alternative cell sources for CAR-neutrophils.

In view of the foregoing, there remains a need for new materials and methods for targeted cancer immunotherapy, such as for the treatment of brain tumors. It is an object of the present disclosure to provide such materials (e.g., CAR neutrophils) and methods. This and other objects and advantages, as well as inventive features, will be apparent from the description and drawings provided herein.

### SUMMARY

A stage-specific process for manufacturing a population of neutrophils from human pluripotent stem cells (hPSCs) is provided. The method comprises the steps of (a) preparing hPSCs; (b) stimulating said hPSCs with a glycogen synthase kinase 3 β (GSK3β) inhibitor to produce a population of CD34+ hemogenic endothelium cells; (c) stimulating said CD34+ hemogenic endothelium cells with a transforming growth factor β (TGFβ) inhibitor to produce a population of CD45+ hematopoietic cells; and (d) stimulating said CD45+ hematopoietic cells with granulocyte macrophage colony-stimulating factor (GM-CSF) and a retinoic acid receptor agonist to afford a population of CD11b+/CD16+ neutrophils. The hPSCs can comprise human embryonic stem cells (hESCs) and induced pluripotent stem cells (iPSCs). The GSK3β inhibitor can be CHIR99021, CHIR98014, or a similar chemical. The TGFβ inhibitor can be SB431542, A83-01 or a similar chemical. The retinoic acid receptor agonist can be AM80, AM50, or a similar chemical. Step (b) can be carried out in the presence of vascular endothelial growth factor (VEGF). Step (c) can be carried out in the presence of stem cell factor (SCF) and FMS-like tyrosine kinase 3 ligand (FLT3L).

A stage-specific process for manufacturing a population of chimeric antigen receptor (CAR) neutrophils is also provided. The method comprises the steps of (a) knocking a CAR expression gene construct into the AAVS1 safe harbor locus of an adeno-associated virus S1 (AAVS1) plasmid in human pluripotent stem cells (hPSCs) via CRISPR/Cas9-mediated homologous recombination; (b) isolating successfully targeted single cell-derived hPSC colonies or hPSC cell mixture to afford a stable CAR-expressing hPSC cell line; (c) preparing hPSCs from said stable CAR-expressing hPSC cell line; (d) stimulating said hPSCs with a glycogen synthase kinase 3 β (GSK3β) inhibitor to produce a population of CD34+ hemogenic endothelium cells; (e) stimulating said CD34+ hemogenic endothelium cells with a transforming growth factor β (TGFβ) inhibitor to produce a population of CD45+ hematopoietic cells; and (f) stimulating said CD45+ hematopoietic cells with granulocyte macrophage colony-stimulating factor (GM-CSF) and a retinoic acid receptor agonist to afford a population of CAR neutrophils. The process can further comprise the initial steps of (a′) preparing a CAR expression gene construct; and (a″) constructing a AAVS1 plasmid. The CAR can comprise chlorotoxin, the transmembrane domain of CD4, and the intracellular domain of CD3. The CAR can have the amino acid sequence of SEQ ID NO: 2. The CCAR can have the amino acid sequence of SEQ ID NO: 1 or 3. The hPSCs can comprise hESCs, such as H9 or H1, and iPSCs, such as 6-9-9 or 19-9-11. The GSK3β inhibitor can be CHIR99021, CHIR98014, or a similar chemical. The TGFβ inhibitor can be SB431542, A83-01, or a similar chemical. The retinoic acid receptor agonist can be AM80, AM50 or a similar chemical. Step (b) can be carried out in the presence of VEGF. Step (c) can be carried out in the presence of SCF and FLT3L.

Further provided is a process for manufacturing a population of CAR neutrophils from hPSCs comprising the steps of (a) constructing a PiggyBac transposon plasmid comprising a CAR expression gene; (b) delivering the PiggyBac plasmid into a human pluripotent stem cell via nucleofection/electroporation; (c) isolating successfully targeted single cell-derived human pluripotent stem cell (hPSC) colonies or hPSC cell mixture for stable CAR-expressing hPSC lines; and (d) producing CAR-expressing neutrophils according to the above stage-specific process for manufacturing a population of neutrophils from hPSCs. The hPSCs can comprise hESCs and iPSCs. The hESCs can comprise H9, H1 or other human embryonic stem cells. The iPSCs can comprise 6-9-9, 19-9-11 or other induced pluripotent stem cells. The CAR can comprise chlorotoxin, the transmembrane domain of CD4, and the intracellular domain of CD3. The CAR can have the amino acid sequence of SEQ ID NO: 2. The CAR has an amino acid sequence of SEQ ID NO: 1 or 3.

Still further provided are engineered neutrophil cell lines from hPSCs. In an embodiment the engineered neutrophil cell line comprises a CAR having the amino acid sequence of SEQ ID NO: 1. In another embodiment the engineered neutrophil cell line comprises a CAR comprising chlorotoxin, the transmembrane domain of CD4, and the intracellular domain of CD3ζ, such as a CAR having the amino acid sequence of SEQ ID NO: 2. In yet another embodiment the engineered neutrophil cell line comprises a CAR having the amino acid sequence of SEQ ID NO: 3.

Even still further provided is a pharmaceutical composition. The pharmaceutical composition comprises a population of isolated CAR neutrophils obtained in accordance with an above-described method or a population of neutrophils from an above-described cell line. The pharmaceutical composition further comprises a pharmaceutically acceptable carrier.

A method of treating cancer in a subject in need thereof is also provided. The method comprises administering to the subject a therapeutically effective amount of (a) a population of neutrophils obtained in accordance with an above-described method or a pharmaceutical composition comprising same and a pharmaceutically acceptable carrier, or (b) a population of neutrophils from above-described cell line or a pharmaceutical composition comprising same and a pharmaceutically acceptable carrier. The cancer can be a brain tumor. The brain tumor can be a glioma. The glioma can be a glioblastoma. The cancer can express the protein matrix metallopeptidase 2. The population of neutrophils or the pharmaceutical composition comprising same can be administered systemically or intracranially.

Finally, a kit for preparing neutrophils from hPSCs is provided. The kit comprises: (a) a glycogen synthase kinase 3 β (GSK3β) inhibitor, optionally as part of a medium; (b) vascular endothelial growth factor (VEGF), optionally as part of a medium; (c) a transforming growth factor β (TGFβ) inhibitor, optionally as part of a medium; (d) granulocyte-macrophage colony-stimulating factor (GM-CSF), interleukin 3 (IL-3), and interleukin 6 (IL-6), optionally as part of a medium; and (e) granulocyte colony-stimulating factor (G-CSF) and a retinoic acid agonist, optionally as part of a medium. The kit can further comprise SCF and Flt3: FMS-like tyrosine kinase 3/fetal liver kinase 2 (Flt3) ligand in (c) and (d) and GlutaMAX and ExCyte in (e).

### BRIEF DESCRIPTION OF FIGURES

FIGS. 1A-1G: hPSC-derived neutrophils adopt a molecular and functional phenotype similar to primary neutrophils. (FIG. 1A) Schematic of optimized neutrophil differentiation from hPSCs under chemically-defined conditions. (FIG. 1B) Representative bright-field images of cellular morphology at the indicated days: D0 hPSCs, D3 mesoderm, D6 hemogenic endothelium, D12 hematopoietic stem and progenitor cells (HSPCs), D15 myeloid progenitor cells, and D21 neutrophils. Scale bars, 100 μm. (FIG. 1C) Flow cytometric analysis of generated neutrophils. Plots show unstained control (blue) and specific antibody (red) histograms. Primary peripheral blood (PB) neutrophils were used as a positive control. (FIG. 1D) Phagocytosis of pHrodo Green E. coli particles by hPSC-derived neutrophils. (FIG. 1E) Transwell migration analysis of the hPSC-derived neutrophils in the absence or presence of chemoattractant (10 nM and 100 nM of fMLP). Data are represented as mean±s.d. of three independent replicates, *p<0.05. (FIG. 1F) Representative tracks, mean velocity, and chemotaxis index of human PB and hPSC-derived neutrophils during chemotaxis are shown. (FIG. 1G) Reactive oxygen species (ROS) production of hPSC-derived and primary PB neutrophils with or without phorbol 12-myristate 13-acetate (PMA) treatment. Data are represented as mean±s.d. of three independent replicates, *p<0.05.

FIGS. 2A-2H: Neutrophils derived from chimeric antigen receptor (CAR) knock-in hPSCs display enhanced antitumor cytotoxicity. (FIG. 2A-2B) Schematic of CLTX-T-CAR construct and targeted knock-in strategy at the AAVS1 safe harbor locus are shown in (FIG. 2A). CLTX-T-CAR is composed of a signal peptide (SP), a glioblastoma-targeting extracellular domain chlorotoxin (CLTX), an Fc domain IgG4 (SmP), a transmembrane domain CD4-tm, and an intracellular signal transduction domain CD3ζ. Vertical arrow indicates the AAVS1 targeting sgRNA. Red and blue horizontal arrows indicate primers for assaying targeting efficiency and homozygosity, respectively. (FIG. 2B) PCR genotyping of hPSC clones after puromycin selection is shown, and the expected PCR product for correctly targeted AAVS1 site is 991 bp (red arrow) with an efficiency of 4 clones from a total of 5. A homozygosity assay was performed on the knock-in clones, and those without ˜240 bp PCR products were homozygous (blue arrow). (FIG. 2C-2D) Schematic of IL-13-T-CAR construct and targeted knock-in strategy at the AAVS1 safe harbor locus were shown in (FIG. 2C). IL-13-T-CAR is composed of a signal peptide (SP), an extracellular domain TQM-13, an Fc domain IgG4 (SmP), a transmembrane domain CD4-tm, and an intracellular signal transduction domain CD3ζ. (FIG. 2D) PCR genotyping of hPSC clones after puromycin selection is shown, and the expected PCR product for correctly targeted AAVS1 site is 991 bp (red arrow) with an efficiency of 15 clones from a total of 24. A homozygosity assay was performed on the knock-in clones, and those without ˜240 bp PCR products were homozygous (blue arrow). (FIG. 2E) Representative RT-PCR analysis of IL-13 and CLTX-IgG4 expression on wildtype and CAR knock-in hPSCs is shown. (FIG. 2F) Cytotoxicity assays against U87MG glioblastoma were performed at different ratios of neutrophil-to-tumor target using indicated neutrophils. Data are represented as mean±s.d. of three independent replicates, *p<0.05. (FIG. 2G) The reactive oxygen species (ROS) generation of different neutrophils co-cultured with or without U87MG cells was measured. (FIG. 2H) The cytotoxicity ability of CLTX-T-CAR hPSC-neutrophils against various tumor cells at a ratio of 10:1 are shown. Glioblastoma U87MG cell line, primary adult GBM43, and pediatric SJ-GBM2 cells were employed. Data are represented as mean±s.d. of three independent replicates. *p<0.05, glioblastoma versus non-glioblastoma tumor.

FIGS. 3A-3G: CLTX-T-CAR hPSC-neutrophils kill the glioblastoma cells by trogoptosis. (FIG. 3A-3C) Representative images of immunological synapses indicated by polarized F-actin accumulation at the interface between CAR-neutrophils and tumor cell were shown in (A) and the numbers of immunological synapses formed between indicated neutrophils and tumor cells were quantified in (FIG. 3B). Neu: neutrophils; Tu: tumor cells. Scale bars, 10 μm. (FIG. 3C) The numbers of immunological synapses formed between CLTX-T-CAR hPSC-neutrophils and indicated cells were quantified. Data are represented as mean±s.d. of three independent replicates. (FIG. 3D-3E) Trogocytosis of glioblastoma cells by neutrophils led to a reduction in cancer cell cytoplasmic labeling in a time-dependent manner Representative brightfield (bright) and fluorescent images of the trogoptotic disruption of Calcein-AM-labeled tumor cells (FIG. 3D), and flow cytometric analysis of CLTX-T-CAR hPSC-neutrophils during tumor trogocytosis (FIG. 3E) were shown. Scale bars, 10 μm. (FIG. 3F) Reactive oxygen species (ROS) generation during the trogoptotic disruption of tumor cells by CLTX-T-CAR hPSC-derived neutrophils was also quantified. (FIG. 3G) Schematic illustration of the trogoptotic disruption of tumor cells by CLTX-T-CAR hPSC-derived neutrophils.

FIGS. 4A-4F: CLTX-T-CAR hPSC-neutrophils specifically bind to glioblastoma via membrane protein MMP2. (FIG. 4A) Schematic of all-in-one Cas13d PiggyBac Transposon platform for inducible knockdown of targeted genes in tumor cells. Tumor cells transfected with CLCN3- (FIG. 4B), ANXA2- (FIG. 4C), and MMP2- (FIG. 4D) targeting Cas13d gRNAs were subjected to RT-PCR analysis with or without doxycycline (DOX) treatment. Anti-tumor cytotoxicity and relative ROS generation of CLTX-T-CAR hPSC-neutrophils against these modified tumor cells were quantified at a neutrophil-to-target ratio of 10:1 with or without DOX treatment. Wildtype tumor cells were used as a positive control. Data are represented as mean±s.d. of three independent replicates. (FIG. 4E) Total and phospho-protein analysis of the Syk-Erk signaling pathway in cell lysates of CLTX-T-CAR and CLTX-NK-CAR hPSC-neutrophils via western blot was performed with or without co-incubation of tumor cell. (FIG. 4F) Schematic illustration of activated cellular Syk-Erk signaling pathway in CAR-neutrophils after binding to MMP2.

FIGS. 5A-5H: Functional evaluation of CLTX-T-CAR hPSC-neutrophils using glioblastoma (GBM) microenvironment mimicking models in vitro. (FIG. 5A) Schematic of in vitro blood-brain-barrier (BBB) model. Transwell migration analysis of wildtype and CLTX-T-CAR hPSC-neutrophils with or without 100 nM of fMLP treatment (FIG. 5B), and their anti-GBM cytotoxicity (FIG. 5C) were assessed in the BBB model. Schematic (FIG. 5D) and quantification (FIG. 5E) of second migration of different hPSC-neutrophils through BBB were shown. (FIG. 5F) Schematic of neutrophil-infiltrated three-dimensional (3D) tumor model in vitro. (FIG. 5G) Representative fluorescent images and quantification of infiltrated wildtype and CLTX-T-CAR hPSC-neutrophils in the 3D tumor models were shown. DAPI was used to stain the cell nuclear and CD45 was used to stain neutrophils. Data are represented as mean±s.d. of three independent replicates, *p<0.05. (FIG. 5H) Live/dead staining of the 3D tumor model was performed after 24 hr of neutrophil infiltration and the corresponding tumor-killing efficiency was quantified. Data are represented as mean±s.d. of three independent replicates, *p<0.05. Scale bars, 200 μm.

FIGS. 6A-6E: In situ anti-tumor activity of CLTX-T-CAR hPSC-neutrophils and -natural killer (NK) cells evaluated via intratumoural injection. (FIG. 6A) Schematic of intratumoural injection of indicated hPSC-neutrophils or NK cells for in vivo anti-tumor cytotoxicity study. 5×10⁵ luciferase (Luci)-expressing U87MG cells were stereotactically implanted into the right forebrain of NSG mice. After 3 hr, PBS, 5×10⁶ wildtype or CLTX-T-CAR hPSC-neutrophils, 5×10⁶ wildtype or CLTX-NK-CAR hPSC-NK cells were injected to the same position. Time-dependent tumor burden was determined (FIG. 6B) and quantified (FIG. 6C) by bioluminescent imaging (BLI) at the indicated days. Data are mean±s.d. for the mice in (B). *n=3. (FIG. 6D) Body weights of indicated experimental mice were measured and recorded weekly. (FIG. 6E) Kaplan-Meier curve demonstrating survival of the experimental groups was shown.

FIGS. 7A-7H: In vivo anti-tumor activities of hPSC-derived CLTX-T-CAR neutrophils and CLTX-NK-CAR NK cells were assessed via intravenous injection. (FIG. 7A) Schematic of intravenous injection of CAR-neutrophils and/or CAR-NK cells for in vivo anti-tumor cytotoxicity study. 5×105 luciferase (Luci)-expressing U87MG cells were stereotactically implanted into the right forebrain of NSG mice. After 4 days, mice were intravenously treated with PBS, 5×106 wildtype neutrophil, CAR-neutrophils, wildtype NK cells, and/or CAR-NK cells weekly for about a month. Time-dependent tumor burden was determined (FIG. 7B) and quantified (FIG. 7C) by bioluminescent imaging (BLI) at the indicated days. Data are mean±s.d. for the mice in (B) (n=6). (FIG. 7D) Kaplan-Meier curve demonstrating survival of the experimental groups was shown. Organs harvested from dead mice were subjected for bioluminescent imaging (FIG. 7E) and quantified in (FIG. 7F). Data are mean±s.d. for the mice in (FIG. 7E) (n=6). (FIG. 7G) Levels of human tumor necrosis factor-α (TNF α) and IL-6 in mouse peripheral blood were measured by ELISA. (FIG. 7H) Wildtype and CAR-neutrophils were isolated from mouse blood 24 hour after systemic injection, and subjected for RT-PCR analysis of anti-tumor N1 and pro-tumor N2 markers.

FIGS. 8A-8J: Generation of aorta-like CD34+SOX17+ hemogenic endothelium (HE) and hematopoietic stem and progenitor cells (HSPCs). (FIG. 8A) Schematic of HE and HSPC generation from hPSCs. (FIG. 8B-8C) hPSC-derived day 5 cultures were subjected to flow cytometry analysis for CD34/SOX17 and quantified in (FIG. 8C). (FIG. 8D) Representative images of immunostaining for SOX17 and VEcad on day 5. Scale bars, 50 μm. (FIG. 8E-8H) hPSCs were differentiated as illustrated in (FIG. 8A). Representative flow plots of CD45/CD43 expression in H9 hPSCs at different time points were shown in (FIG. 8E) and quantified in (FIG. 8F). Representative flow plots of CD44/CD43 in H9 (FIG. 8G) cultures and CD34/RUNC1c (FIG. 8H) in H9 RUNX1c-GFP (Ng et al., 2016) cultures at the indicated days were shown. (FIG. 8I-8J) Cell viability before and after froze was assessed by flow cytometry with Calcein-AM stain.

FIGS. 9A-9I: Multipotent evaluation of hPSC-derived myeloid progenitors. (FIG. 9A-9C) Schematic of colony forming unit-macrophage (CFU-M) and -granulocyte-macrophage (CFU-GM) analysis at the indicated days during differentiation was shown in (FIG. 9A). Representative images of G and GM colonies formed by hPSC-derived myeloid progenitors collected at the indicated days were shown in (FIG. 9B) and quantified in (FIG. 9C). Data are represented as mean±s.d. of three independent replicates. Scale bars, 100 μm. (FIG. 9D-9E) Monocyte/Macrophage differentiation potential of hPSC-derived myeloid progenitor cells was assessed by applying GM-CSF treatment. Representative flow plots of CD14 and CD45 under different culture conditions were shown in (FIG. 9D) and quantified in (FIG. 9E). (FIG. 9F-9H) Schematic of neutrophil differentiation potential evaluation of hPSC-derived myeloid progenitors at the indicated days with G-CSF and AM580 treatment was shown in (FIG. 9F). The expressions of CD11b and CD16 in cell cultures collected at the indicated days were assessed by flow cytometry (FIG. 9G) and quantified in (FIG. 9H). Representative Wright-Giemsa staining of the hPSC-derived and primary peripheral blood (PB) neutrophils were shown in (FIG. 9I). Scale bars, 10 μm. Data are represented as mean±s.d. of three independent replicates.

FIGS. 10A-10F: Construction and characterization of CAR knockin hPSCs. (FIG. 10A-10B) Schematic of CLTX-NK-CAR construct and targeted knock-in strategy at the AAVS1 safe harbor locus (FIG. 10A). CLTX-NK-CAR is composed of a signal peptide (SP), a glioblastoma-targeting extracellular domain chlorotoxin (CLTX), an Fc domain IgG4 (SmP), a transmembrane domain NKG2D-tm, a co-stimulatory domain 2B4, and an intracellular signal transduction domain CD3. Vertical arrow indicates the AAVS1 targeting sgRNA. Red and blue horizontal arrows indicate primers for assaying targeting efficiency and homozygosity, respectively. (FIG. 10B) PCR genotyping of hPSC clones after puromycin selection is shown, and expected PCR product for correctly targeted AAVS1 site is 991 bp (red arrow) with an efficiency of 4 clones from a total of 13. A homozygosity assay was performed on the knock-in clones, and those without ˜240 bp PCR products were homozygous (blue arrow). (FIG. 10C-10F) Phenotypical and functional evaluation of CAR hPSCs was performed. Representative fluorescent images (FIG. 10C) and flow plots (FIG. 10D) of OCT4 and SSEA4 expression were shown. Scale bars, 50 μm. (FIG. 10E-10F) Wildtype hPSCs were differentiated into three germ layer lineages: mesoderm, endoderm, and ectoderm. Representative immunostaining images of cTnT, HNF4A and β-III tubulin were shown in (FIG. 10E). Scale bars, 100 μm. (FIG. 10F) CAR-neutrophils were derived from CLTX-T-CAR hPSCs, and incubated with wildtype H9 hPSCs, hPSC-derived mesoderm, endoderm and ectoderm at the indicated neutrophil-to-target ratios. The numbers of viable cells were quantified. Data are represented as mean±s.d. of three independent replicates.

FIGS. 11A-11C: Inducible gene knockdown in tumor cells using PiggyBac transposon-based Cas13d system. Glioblastoma U87MG cells were transfected with the indicated all-in-one Cas13d gRNA constructs along with the hyPBase plasmid. Representative brightfield and fluorescent images (FIG. 11A), and flow plots (FIG. 11B) of eGFP signal after doxycycline (DOX) treatment were shown. Scale bars, 100 μm. (FIG. 11C) CLTX-T-CAR hPSC-derived neutrophils were incubated with the indicated gene knockdown glioblastoma cells in the presence or absence of DOX Immune synapse formation between neutrophils and indicated tumor cells were quantified and shown in (FIG. 11C). Wildtype U87MG cells were used as a negative control.

FIGS. 12A-12G: In vivo anti-tumor activity of CLTX-T-CAR neutrophils and CLTX-NK-CAR NK cells was assessed via intravenous injection. (FIG. 12A) Schematic of intravenous injection of Cy5-labeled hPSC-neutrophils for in vivo cell tracking study. 5×105 luciferase (Luci)-expressing U87MG cells were stereotactically implanted into the right forebrain of NSG mice. After 4 days, mice were intravenously treated with PBS, 5×106 Cy5-labeled wildtype or CLTX-T-CAR hPSC-neutrophils. Time-dependent biodistribution of Cy5+ neutrophils in whole body (FIG. 12B), brain (FIG. 12C) and other organs (FIG. 12D) was determined and quantified by fluorescence imaging at the indicated hours. (FIG. 12E) Body weight of the experimental mice was measured weekly. (FIG. 12F) Representative brightfield and H&E staining images of glioblastoma xenografts isolated from indicated mice were shown. Tumor area were circulated in dashed line. (FIG. 12G) Wildtype and CAR-neutrophils, isolated from mouse blood 24 hour after systemic injection, were subjected for RTPCR analysis of anti-tumor N1 and pro-tumor N2 markers and quantified.

FIGS. 13A-13C: Anti-PSMA CAR-neutrophils derived from hPSCs specifically recognize and kill cancerous lines. (FIG. 13A) Schematic diagrams of PSMA-CAR design and knock-in strategy via Cas9-mediated homology-directed repair (HDR) at the endogenous AAVS1 safe harbor locus. PSMA-CAR is composed of signal peptide, anti-PSMA J591 scFV or nanobody, IgG4-Fc (EQ), CD4 transmembrane (tm) and CD3ζ (CD3z). (FIG. 13B) Genotyping of CAR knockin in hPSCs with a target efficiency of 12 clones from a total of 13 and 13 clones from a total of 15, respectively. (FIG. 13C) CAR-neutrophils were co-cultured with U87MG glioblastoma (GBM) and LNCaP prostate cancer cells at indicated cell ratios for 16 hr and the cytotoxicity of neutrophils was calculated.

FIG. 14 depicts the CLTX NK-CAR plasmid map.

FIG. 15 depicts the CLTX T-CAR plasmid map.

FIG. 16 depicts the IL-13 T-CAR plasmid map.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure seeks to provide materials and methods for more effective and rapid generation of neutrophils. The innovative platform employs a robust, stage-specific differentiation process, which enables the large-scale production of “off-the-shelf,” functional neutrophils, which can present superior and specific cytotoxicity against various tumor cells in vitro. Importantly, these neutrophils demonstrate enhanced brain tumor cell killing ability. Furthermore, chlorotoxin (CLTX) T-CAR modified neutrophils demonstrate minimal cytotoxicity against normal cells, thereby offering great potential for future clinical applications in glioblastoma therapy. The neutrophils can be employed in various clinical therapeutic applications including, but not limited to, the delivery of nanomedicines to tumor cells and the targeted immunotherapy of solid cancers (e.g., tumors) via chimeric antigen receptors (CARs).

### Definitions

The following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 20%, within 10%, within 5%, or within 1% of a stated value or of a stated limit of a range.

The term “substantially” can allow for a degree of variability in a value or range, for example, within 80%, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limited to the disclosure set forth under a given section heading. Thus, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies (for example, between sequences set forth in this document and those set forth in the Sequence Listing), the usage in this document controls.

Methods of Manufacturing a Population of Neutrophils

A stage-specific process for manufacturing a population of neutrophils from hPSCs is provided. The method comprises the steps of (a) preparing hPSCs; (b) stimulating said hPSCs with a glycogen synthase kinase 3 β (GSK3β) inhibitor to produce a population of CD34+ hemogenic endothelium cells; (c) stimulating said CD34+ hemogenic endothelium cells with a transforming growth factor β (TGFβ) inhibitor to produce a population of CD45+ hematopoietic cells; and (d) stimulating said CD45+ hematopoietic cells with granulocyte macrophage colony-stimulating factor (GM-CSF) and a retinoic acid receptor agonist to afford a population of CD11b+/CD16+ neutrophils. The hPSCs can comprise human embryonic stem cells (hESCs) and induced pluripotent stem cells (iPSCs). The GSK3β inhibitor can be CHIR99021, CHIR98014, or a similar chemical. The TGFβ inhibitor can be SB431542, A83-01 or a similar chemical. The retinoic acid receptor agonist can be AM80, AM50, or a similar chemical. Step (b) can be carried out in the presence of vascular endothelial growth factor (VEGF). Step (c) can be carried out in the presence of stem cell factor (SCF) and FMS-like tyrosine kinase 3 ligand (FLT3L).

A stage-specific process for manufacturing a population of chimeric antigen receptor (CAR) neutrophils is also provided. “CAR neutrophils” are neutrophils that have been modified through molecular biological methods to express a CAR on the surfaces of the neutrophils. The CAR is a polypeptide having a pre-defined binding specificity to a desired target, such as matrix metallopeptidase 2 (MMP2), e.g., MMP2 on a glioma, such as a glioblastoma. The CAR can include other domains, such as various signaling domains, costimulatory domains, spacers and/or hinges.

Use of terms and phrases with regard to CAR binding specificity, such as “binds with specificity,” “binds with high affinity,” or “specifically” or “selectively” binds, indicates a binding reaction between a CAR, such as a CAR comprising CLTX, on a neutrophil and a target molecule, such as a protein, e.g., a receptor, an enzyme (e.g., MMP2) or a cell-surface marker, that is present on a targeted cell, such as a cancerous cell, e.g., a cell of which a tumor is comprised, or other diseased cell. Thus, under binding conditions that are conducive to, facilitate or otherwise promote binding of a CAR neutrophil with a target molecule that is present on a targeted cell, such as a cancerous cell or other diseased cell, the CAR neutrophil does not bind significantly, if at all, to other molecules, such as proteins, e.g., receptors, enzymes, and cell-surface markers, present or normal, healthy cells. Specific binding or binding with high affinity can be at least 25% greater, more often at least 50% greater, most often at least 100% (2-fold) greater, normally at least ten times greater, more normally at least 20-times greater, and most normally at least 100-times greater than the binding of any other non-targeted molecule.

A CAR can be produced by any means known in the art, though preferably it is produced using recombinant DNA techniques. A nucleic acid sequence encoding the several regions of the CAR can be prepared and assembled into a complete coding sequence by standard techniques of molecular cloning (genomic library screening, PCR, primer-assisted ligation, site-directed mutagenesis, and gene editing techniques, such as CRISP, etc.). Such techniques are described in detail in Sambrook et al., “Molecular Cloning: A Laboratory Manual,” 3rd Edition, Cold Spring Harbor Laboratory Press, (2001), and Green and Sambrook, “Molecular Cloning: A Laboratory Manual,” 4th Edition, Cold Spring Harbor Laboratory Press, (2012), which are both incorporated herein by reference in their entireties (collectively, the “Protocols”).

The resulting coding region can be inserted into an expression vector for subsequent introduction into a recipient cell, such as a hPSC. The term “vector” means any nucleic acid that functions to carry, harbor, or express a nucleic acid of interest. Nucleic acid vectors can have specialized functions, such as expression, packaging, pseudotyping, or transduction. Vectors can also have manipulatory functions if adapted for use as a cloning or shuttle vector. The structure of the vector can include any desired form that is feasible to make and desirable for a particular use. Such forms can include, for example, circular forms such as plasmids and phagemids, as well as linear or branched forms. A nucleic acid vector can be composed of, or example, DNA or RNA, as well as contain partially or fully, nucleotide derivatives, analogs or mimetics. Such vectors can be obtained from natural sources, produced recombinantly or chemically synthesized.

CAR expression can be driven using any suitable promoter, such as exemplified herein. Examples of promoters include, but are not limited to, various constitutive and inducible promoters, such as a constitutive CAG promoter, an EF1a promoter, a UBC constitutive promoter, or a Teton-3G inducible promoter.

The placement of the recognition region in the fusion protein will generally be such that display of the region on the exterior of the cell is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD3ζ transmembrane domain), and a hinge and/or a spacer domain that imparts flexibility to the recognition region and allows strong binding to the targeting moiety.

Any suitable method as known in the art and exemplified herein can be used to deliver a CAR-encoding nucleic acid, such as a plasmid, into hPSCs. Examples of methods include, but are not limited to, nucleofection/electroporation, transfection via Lipofectamine Stem (ThermoFisher, STEM00001) or similar transfection reagents, or lentivirus, retrovirus, sleeping beauty, piggyback (transposon/transposase systems including a non-viral mediated CAR gene delivery system) or adeno-associated virus (AAV)-mediated delivery. Illustrated is a method using plasmids and CRISP/Cas9, and transposons, ribonucleoproteins and double-stranded DNAs also can be used to integrate a CAR into the hPSC genome, such as at an AAVS1 safe harbor locus or a CLYBL locus.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it is positioned to facilitate translation. Generally, “operably linked” means that the DNA sequences being linked are contiguous and, in the case of leader, contiguous and in a reading phase. However, enhancers do not necessarily have to be contiguous. Linking may be accomplished by ligation at convenient restriction sites. If such sites do not exist, synthetic oligonucleotide adaptors or linkers may be used in accordance with conventional practice.

The stage-specific process for manufacturing a population of chimeric antigen receptor (CAR) neutrophils comprises the steps of (a) knocking a CAR expression gene construct into the AAVS1 safe harbor locus of an adeno-associated virus S1 (AAVS1) plasmid in human pluripotent stem cells (hPSCs) via CRISPR/Cas9-mediated homologous recombination; (b) isolating successfully targeted single cell-derived hPSC colonies or hPSC cell mixture to afford a stable CAR-expressing hPSC cell line; (c) preparing hPSCs from said stable CAR-expressing hPSC cell line; (d) stimulating said hPSCs with a GSK3β inhibitor to produce a population of CD34+ hemogenic endothelium cells; (e) stimulating said CD34+ hemogenic endothelium cells with a transforming growth factor β (TGFβ) inhibitor to produce a population of CD45+ hematopoietic cells; and (f) stimulating said CD45+ hematopoietic cells with granulocyte macrophage colony-stimulating factor (GM-CSF) and a retinoic acid receptor agonist to afford a population of CAR neutrophils. The process can further comprise the initial steps of (a′) preparing a CAR expression gene construct; and (a″) constructing a AAVS1 plasmid. The CAR can comprise CLTX, the transmembrane domain of CD4, and the intracellular domain of CD3ζ. The CAR can have the amino acid sequence of SEQ ID NO: 2. The CCAR can have the amino acid sequence of SEQ ID NO: 1 or 3. The hPSCs can comprise hESCs, such as H9 or H1, and iPSCs, such as 6-9-9 or 19-9-11. The GSK3β inhibitor can be CHIR99021, CHIR98014, or a similar chemical. The TGFβ inhibitor can be SB431542, A83-01, or a similar chemical. The retinoic acid receptor agonist can be AM80, AM50 or a similar chemical. Step (b) can be carried out in the presence of VEGF. Step (c) can be carried out in the presence of SCF and FLT3L.

Further provided is a process for manufacturing a population of CAR neutrophils from hPSCs comprising the steps of (a) constructing a PiggyBac transposon plasmid comprising a CAR expression gene; (b) delivering the PiggyBac plasmid into a human pluripotent stem cell via nucleofection/electroporation; (c) isolating successfully targeted single cell-derived human pluripotent stem cell (hPSC) colonies or hPSC cell mixture for stable CAR-expressing hPSC lines; and (d) producing CAR-expressing neutrophils according to the above stage-specific process for manufacturing a population of neutrophils from hPSCs. The hPSCs can comprise hESCs and iPSCs. The hESCs can comprise H9, H1 or other human embryonic stem cells. The iPSCs can comprise 6-9-9, 19-9-11 or other induced pluripotent stem cells. The CAR can comprise CLTX, the transmembrane domain of CD4, and the intracellular domain of CD3ζ. The CAR can have the amino acid sequence of SEQ ID NO: 2. The CAR can have the amino acid sequence of SEQ ID NO: 1 or 3.

SEQ ID NO: 1 is CLTX NK-CAR amino acid sequence:
(SEQ ID NO: 1)
MetLeuLeuLeuValThrSerLeuLeuLeuCysGluLeuProHisProAlaPheLeuLeu
IleProMetCysMetProCysPheThrThrAspHisGlnMetAlaArgLysCysAspAsp
CysCysGlyGlyLysGlyArgGlyLysCysTyrGlyProGlnCysLeuCysArgGluSer
LysTyrGlyProProCysProProCysProAlaProGluPheLeuGlyGlyProSerVal
PheLeuPheProProLysProLysAspThrLeuMetIleSerArgThrProGluValThr
CysValValValAspValSerGlnGluAspProGluValGlnPheAsnTrpTyrValAsp
GlyValGluValHisAsnAlaLysThrLysProArgGluGluGlnPheAsnSerThrTyr
ArgValValSerValLeuThrValLeuHisGlnAspTrpLeuAsnGlyLysGluTyrLys
CysLysValSerAsnLysGlyLeuProSerSerIleGluLysThrIleSerLysAlaLys
GlyGlnProArgGluProGlnValTyrThrLeuProProSerGlnGluGluMetThrLys
AsnGlnValSerLeuThrCysLeuValLysGlyPheTyrProSerAspIleAlaValGlu
TrpGluSerAsnGlyGlnProGluAsnAsnTyrLysThrThrProProValLeuAspSer
AspGlySerPhePheLeuTyrSerArgLeuThrValAspLysSerArgTrpGlnGluGly
AsnValPheSerCysSerValMetHisGluAlaLeuHisAsnHisTyrThrGlnLysSer
LeuSerLeuSerLeuGlyLysProPhePhePheCysCysPheIleAlaValAlaMetGly
IleArgPheIleIleMetValThrTrpArgArgLysArgLysGluLysGlnSerGluThr
SerProLysGluPheLeuThrIleTyrGluAspValLysAspLeuLysThrArgArgAsn
HisGluGlnGluGlnThrPheProGlyGlyGlySerThrIleTyrSerMetIleGlnSer
GlnSerSerAlaProThrSerGlnGluProAlaTyrThrLeuTyrSerLeuIleGlnPro
SerArgLysSerGlySerArgLysArgAsnHisSerProSerPheAsnSerThrIleTyr
GluValIleGlyLysSerGlnProLysAlaGlnAsnProAlaArgLeuSerArgLysGlu
LeuGluAsnPheAspValTyrSerArgValLysPheSerArgSerAlaAspAlaProAla
TyrGlnGlnGlyGlnAsnGlnGlnLeuTyrAsnGluLeuAsnLeuGlyArgArgGluGlu
TyrAspValLeuAspLysArgArgGlyArgAspProGluMetGlyGlyLysProArgArg
LysAsnProGlnGluGlyLeuTyrAsnGluLeuGlnLysAspLysMetAlaGluAlaTyr
SerGluIleGlyMetLysGlyGluArgArgArgGlyLysGlyHisAspGlyLeuTyrGln
GlyLeuSerThrAlaThrLysAspThrTyrAspAlaLeuHisMetGlnAlaLeuProPro
Arg.
SEQ ID NO: 2 is CLTX T-CAR amino acid sequence:
(SEQ ID NO: 2)
MetLeuLeuLeuValThrSerLeuLeuLeuCysGluLeuProHisProAlaPheLeuLeu
IleProMetCysMetProCysPheThrThrAspHisGlnMetAlaArgLysCysAspAsp
CysCysGlyGlyLysGlyArgGlyLysCysTyrGlyProGlnCysLeuCysArgGluSer
LysTyrGlyProProCysProProCysProAlaProGluPheLeuGlyGlyProSerVal
PheLeuPheProProLysProLysAspThrLeuMetIleSerArgThrProGluValThr
CysValValValAspValSerGlnGluAspProGluValGlnPheAsnTrpTyrValAsp
GlyValGluValHisAsnAlaLysThrLysProArgGluGluGlnPheAsnSerThrTyr
ArgValValSerValLeuThrValLeuHisGlnAspTrpLeuAsnGlyLysGluTyrLys
CysLysValSerAsnLysGlyLeuProSerSerIleGluLysThrIleSerLysAlaLys
GlyGlnProArgGluProGlnValTyrThrLeuProProSerGlnGluGluMetThrLys
AsnGlnValSerLeuThrCysLeuValLysGlyPheTyrProSerAspIleAlaValGlu
TrpGluSerAsnGlyGlnProGluAsnAsnTyrLysThrThrProProValLeuAspSer
AspGlySerPhePheLeuTyrSerArgLeuThrValAspLysSerArgTrpGlnGluGly
AsnValPheSerCysSerValMetHisGluAlaLeuHisAsnHisTyrThrGlnLysSer
LeuSerLeuSerLeuGlyLysMetAlaLeuIleValLeuGlyGlyValAlaGlyLeuLeu
LeuPheIleGlyLeuGlyIlePhePheArgValLysPheSerArgSerAlaAspAlaPro
AlaTyrGlnGlnGlyGlnAsnGlnGlnLeuTyrAsnGluLeuAsnLeuGlyArgArgGlu
GluTyrAspValLeuAspLysArgArgGlyArgAspProGluMetGlyGlyLysProArg
ArgLysAsnProGlnGluGlyLeuTyrAsnGluLeuGlnLysAspLysMetAlaGluAla
TyrSerGluIleGlyMetLysGlyGluArgArgArgGlyLysGlyHisAspGlyLeuTyr
GlnGlyLeuSerThrAlaThrLysAspThrTyrAspAlaLeuHisMetGlnAlaLeuPro
ProArg.
SEQ ID NO: 3 is IL-13 T-CAR amino acid sequence:
(SEQ ID NO: 3)
MetLeuLeuLeuValThrSerLeuLeuLeuCysGluLeuProHisProAlaPheLeuLeu
IleProGlyProValProProSerThrAlaLeuArgTyrLeuIleGluGluLeuValAsn
IleThrGlnAsnGlnLysAlaProLeuCysAsnGlySerMetValTrpSerIleAsnLeu
ThrAlaGlyMetTyrCysAlaAlaLeuGluSerLeuIleAsnValSerGlyCysSerAla
IleGluLysThrGlnArgMetLeuSerGlyPheCysProHisLysValSerAlaGlyGln
PheSerSerLeuHisValArgAspThrLysIleGluValAlaGlnPheValLysAspLeu
LeuLeuHisLeuLysLysLeuPheArgGluGlyArgPheAsnGluSerLysTyrGlyPro
ProCysProProCysProAlaProGluPheLeuGlyGlyProSerValPheLeuPhePro
ProLysProLysAspThrLeuMetIleSerArgThrProGluValThrCysValValVal
AspValSerGlnGluAspProGluValGlnPheAsnTrpTyrValAspGlyValGluVal
HisAsnAlaLysThrLysProArgGluGluGlnPheAsnSerThrTyrArgValValSer
ValLeuThrValLeuHisGlnAspTrpLeuAsnGlyLysGluTyrLysCysLysValSer
AsnLysGlyLeuProSerSerIleGluLysThrIleSerLysAlaLysGlyGlnProArg
GluProGlnValTyrThrLeuProProSerGlnGluGluMetThrLysAsnGlnValSer
LeuThrCysLeuValLysGlyPheTyrProSerAspIleAlaValGluTrpGluSerAsn
GlyGlnProGluAsnAsnTyrLysThrThrProProValLeuAspSerAspGlySerPhe
PheLeuTyrSerArgLeuThrValAspLysSerArgTrpGlnGluGlyAsnValPheSer
CysSerValMetHisGluAlaLeuHisAsnHisTyrThrGlnLysSerLeuSerLeuSer
LeuGlyLysMetAlaLeuIleValLeuGlyGlyValAlaGlyLeuLeuLeuPheIleGly
LeuGlyIlePhePheArgValLysPheSerArgSerAlaAspAlaProAlaTyrGlnGln
GlyGlnAsnGlnGlnLeuTyrAsnGluLeuAsnLeuGlyArgArgGluGluTyrAspVal
LeuAspLysArgArgGlyArgAspProGluMetGlyGlyLysProArgArgLysAsnPro
GlnGluGlyLeuTyrAsnGluLeuGlnLysAspLysMetAlaGluAlaTyrSerGluIle
GlyMetLysGlyGluArgArgArgGlyLysGlyHisAspGlyLeuTyrGlnGlyLeuSer
ThrAlaThrLysAspThrTyrAspAlaLeuHisMetGlnAlaLeuProProArg.
SEQ ID NO: 8 is CLTX CD32 FcγR-CAR amino acid sequence:
(SEQ ID NO: 8)
MetLeuLeuLeuValThrSerLeuLeuLeuCysGluLeuProHisProAlaPheLeuLeu
IleProMetCysMetProCysPheThrThrAspHisGlnMetAlaArgLysCysAspAsp
CysCysGlyGlyLysGlyArgGlyLysCysTyrGlyProGlnCysLeuCysArgGluSer
LysTyrGlyProProCysProProCysProAlaProGluPheLeuGlyGlyProSerVal
PheLeuPheProProLysProLysAspThrLeuMetIleSerArgThrProGluValThr
CysValValValAspValSerGlnGluAspProGluValGlnPheAsnTrpTyrValAsp
GlyValGluValHisAsnAlaLysThrLysProArgGluGluGlnPheAsnSerThrTyr
ArgValValSerValLeuThrValLeuHisGlnAspTrpLeuAsnGlyLysGluTyrLys
CysLysValSerAsnLysGlyLeuProSerSerIleGluLysThrIleSerLysAlaLys
GlyGlnProArgGluProGlnValTyrThrLeuProProSerGlnGluGluMetThrLys
AsnGlnValSerLeuThrCysLeuValLysGlyPheTyrProSerAspIleAlaValGlu
TrpGluSerAsnGlyGlnProGluAsnAsnTyrLysThrThrProProValLeuAspSer
AspGlySerPhePheLeuTyrSerArgLeuThrValAspLysSerArgTrpGlnGluGly
AsnValPheSerCysSerValMetHisGluAlaLeuHisAsnHisTyrThrGlnLysSer
LeuSerLeuSerLeuGlyLysIleIleValAlaValValIleAlaThrAlaValAlaAla
IleValAlaAlaValValAlaLeuIleTyrCysArgLysLysArgIleSerAlaAsnSer
ThrAspProValLysAlaAlaGlnPheGluProProGlyArgGlnMetIleAlaIleArg
LysArgGlnLeuGluGluThrAsnAsnAspTyrGluThrAlaAspGlyGlyTyrMetThr
LeuAsnProArgAlaProThrAspAspAspLysAsnIleTyrLeuThrLeuProProAsn
AspHisValAsnSerAsnAsn.
SEQ ID NO: 9 is anti-PSMA nanobody T-CAR amino acid sequence:
(SEQ ID NO: 9)
MetLeuLeuLeuValThrSerLeuLeuLeuCysGluLeuProHisProAlaPheLeuLeu
IleProGluValGlnLeuValGluSerGlyGlyGlyLeuValGlnProGlyGlySerLeu
ThrLeuSerCysAlaAlaSerArgPheMetIleSerGluTyrSerMetHisTrpValArg
GlnAlaProGlyLysGlyLeuGluTrpValSerThrIleAsnProAlaGlyThrThrAsp
TyrAlaGluSerValLysGlyArgPheThrIleSerArgAspAsnAlaLysAsnThrLeu
TyrLeuGlnMetAsnSerLeuLysProGluAspThrAlaValTyrTyrCysAspGlyTyr
GlyTyrArgGlyGlnGlyThrGlnValThrValSerSerGluSerLysTyrGlyProPro
CysProProCysProAlaProGluPheLeuGlyGlyProSerValPheLeuPheProPro
LysProLysAspThrLeuMetIleSerArgThrProGluValThrCysValValValAsp
ValSerGlnGluAspProGluValGlnPheAsnTrpTyrValAspGlyValGluValHis
AsnAlaLysThrLysProArgGluGluGlnPheAsnSerThrTyrArgValValSerVal
LeuThrValLeuHisGlnAspTrpLeuAsnGlyLysGluTyrLysCysLysValSerAsn
LysGlyLeuProSerSerIleGluLysThrIleSerLysAlaLysGlyGlnProArgGlu
ProGlnValTyrThrLeuProProSerGlnGluGluMetThrLysAsnGlnValSerLeu
ThrCysLeuValLysGlyPheTyrProSerAspIleAlaValGluTrpGluSerAsnGly
GlnProGluAsnAsnTyrLysThrThrProProValLeuAspSerAspGlySerPhePhe
LeuTyrSerArgLeuThrValAspLysSerArgTrpGlnGluGlyAsnValPheSerCys
SerValMetHisGluAlaLeuHisAsnHisTyrThrGlnLysSerLeuSerLeuSerLeu
GlyLysMetAlaLeuIleValLeuGlyGlyValAlaGlyLeuLeuLeuPheIleGlyLeu
GlyIlePhePheArgValLysPheSerArgSerAlaAspAlaProAlaTyrGlnGlnGly
GlnAsnGlnGlnLeuTyrAsnGluLeuAsnLeuGlyArgArgGluGluTyrAspValLeu
AspLysArgArgGlyArgAspProGluMetGlyGlyLysProArgArgLysAsnProGln
GluGlyLeuTyrAsnGluLeuGlnLysAspLysMetAlaGluAlaTyrSerGluIleGly
MetLysGlyGluArgArgArgGlyLysGlyHisAspGlyLeuTyrGlnGlyLeuSerThr
AlaThrLysAspThrTyrAspAlaLeuHisMetGlnAlaLeuProProArg.
SEQ ID NO: 10 is anti-PSMA J-591-scFV T-CAR amino acid sequence:
(SEQ ID NO: 10)
MetLeuLeuLeuValThrSerLeuLeuLeuCysGluLeuProHisProAlaPheLeuLeu
IleProGluValGlnLeuValGlnSerGlyAlaGluValLysLysProGlyAlaSerVal
LysIleSerCysLysThrSerGlyTyrThrPheThrGluTyrThrIleHisTrpValLys
GlnAlaSerGlyLysGlyLeuGluTrpIleGlyAsnIleAsnProAsnAsnGlyGlyThr
ThrTyrAsnGlnLysPheGluAspArgAlaThrLeuThrValAspLysSerThrSerThr
AlaTyrMetGluLeuSerSerLeuArgSerGluAspThrAlaValTyrTyrCysAlaAla
GlyTrpAsnPheAspTyrTrpGlyGlnGlyThrThrValThrValSerSerGlySerThr
SerGlyGlyGlySerGlyGlyGlySerGlyGlyGlyGlySerSerAspIleValMetThr
GlnSerProSerSerLeuSerAlaSerValGlyAspArgValThrIleThrCysLysAla
SerGlnAspValGlyThrAlaValAspTrpTyrGlnGlnLysProGlyLysAlaProLys
LeuLeuIleTyrTrpAlaSerThrArgHisThrGlyValProAspArgPheThrGlySer
GlySerGlyThrAspPheThrLeuThrIleSerSerLeuGlnProGluAspPheAlaAsp
TyrPheCysGlnGlnTyrAsnSerTyrProLeuThrPheGlyGlyGlyThrLysLeuGlu
IleLysGluSerLysTyrGlyProProCysProProCysProAlaProGluPheLeuGly
GlyProSerValPheLeuPheProProLysProLysAspThrLeuMetIleSerArgThr
ProGluValThrCysValValValAspValSerGlnGluAspProGluValGlnPheAsn
TrpTyrValAspGlyValGluValHisAsnAlaLysThrLysProArgGluGluGlnPhe
AsnSerThrTyrArgValValSerValLeuThrValLeuHisGlnAspTrpLeuAsnGly
LysGluTyrLysCysLysValSerAsnLysGlyLeuProSerSerIleGluLysThrIle
SerLysAlaLysGlyGlnProArgGluProGlnValTyrThrLeuProProSerGlnGlu
GluMetThrLysAsnGlnValSerLeuThrCysLeuValLysGlyPheTyrProSerAsp
IleAlaValGluTrpGluSerAsnGlyGlnProGluAsnAsnTyrLysThrThrProPro
ValLeuAspSerAspGlySerPhePheLeuTyrSerArgLeuThrValAspLysSerArg
TrpGlnGluGlyAsnValPheSerCysSerValMetHisGluAlaLeuHisAsnHisTyr
ThrGlnLysSerLeuSerLeuSerLeuGlyLysMetAlaLeuIleValLeuGlyGlyVal
AlaGlyLeuLeuLeuPheIleGlyLeuGlyIlePhePheArgValLysPheSerArgSer
AlaAspAlaProAlaTyrGlnGlnGlyGlnAsnGlnGlnLeuTyrAsnGluLeuAsnLeu
GlyArgArgGluGluTyrAspValLeuAspLysArgArgGlyArgAspProGluMetGly
GlyLysProArgArgLysAsnProGlnGluGlyLeuTyrAsnGluLeuGlnLysAspLys
MetAlaGluAlaTyrSerGluIleGlyMetLysGlyGluArgArgArgGlyLysGlyHis
AspGlyLeuTyrGlnGlyLeuSerThrAlaThrLysAspThrTyrAspAlaLeuHisMet
GlnAlaLeuProProArg.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages that are synthetic, naturally occurring, and non-naturally occurring, have similar binding properties as the reference nucleic acid, and are metabolized in a manner similar to the reference nucleotides.

The DNA sequences (SEQ ID Nos: 11-16) that encode corresponding proteins of SEQ ID NOs: 1˜3 and 8˜10 are claimed and fell within the scopes of this disclosure.

The terms “polypeptide,” “peptide,” and “protein” are used interchangeably herein (unless expressly stated otherwise) to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

Further, while the present disclosure illustrates the introduction of CAR into hPSCs followed by differentiation into neutrophils, it will be understood and appreciated by one of ordinary skill in the art that hPSC-derived hemogenic endothelial, hematopoietic progenitor cells, and neutrophils, such as those generated in accordance with a method described herein, can be directly targeted to make CAR-neutrophils.

Human peripheral blood mononuclear cells (PBMCs) are routinely used to drive monocytes for many areas of biomedical research. The logistics of granulocyte collection, however, are complicated. For example, donors need to be pre-treated with granulocyte colony-stimulating factor (G-CSF) or steroid (Gea-Banacloche, Granulocyte transfusions: A concise review for practitioners. Cytotherapy (2017). doi:10.1016/j.jcyt.2017.08.012; Adrover et al., Immunity 50: 390-402.e10 (2019); and Gurlek Gokcebay et al., Granulocyte transfusions in the management of neutropenic fever: A pediatric perspective. Transfusion and Apheresis Science (2018). doi:10.1016/j.transci.2018.02.009). In addition, it is difficult to collect a sufficient number of good quality cells, and storage time is limited to around 24 hours (Ginhoux et al., Developmental pathways and tissue homeostasis, Nature Reviews Immunology (2014). doi:10.1038/nri3671). These factors hamper the utility of granulocyte transfusion for correcting neutropenia and often contribute to the inconclusive results observed in clinical trials (Brok-Volchanskaya et al., Stem Cell Reports 13: 1099-1110 (2019); and Cao et al., Stem Cell Reports 12: 1281-1297 (2019)).

Human pluripotent stem cells (hPSCs) offer the potential to serve as an alternative and scalable source of granulocytes (Seaki et al., A feeder-free and efficient production of functional neutrophils from human embryonic stem cells, Stem Cells (2009). doi:10.1634/stemcells.2007-0980). Although prior studies have demonstrated the feasibility of neutrophil generation from hPSCs, the employment of serum and feeder or embryoid body formation has limited their broader applications (Trump et al., Stem Cells Transl Med 8: 557-567 (2019)).

Previous studies have shown that hPSCs can differentiate into hemogenic endothelium (HE), marked by vascular endothelial cadherin+(VECad+) and CD34+ cells and early hematopoietic progenitors expressing the hematopoietic markers CD43 and CD45. These hematopoietic cells can give rise to erythro-myeloid progenitor (EMP)-like cells with broad erythroid and myeloid differentiation capacity (Ivanovs et al., Development 144: 2323-2337 (2017); Dou et al., Medial HOXA genes demarcate haematopoietic stem cell fate during human development, Nat Cell Biol (2016). doi:10.1038/ncb3354; and Hwang et al., Controlled differentiation of stem cells. Adv Drug Deliv Rev (2008). doi:10.1016/j.addr.2007.08.036). The myeloid progenitors could be subsequently differentiated into mature neutrophils in the presence of G-CSF and the retinoic acid agonist Am 580.

Engineered Neutrophil Cell Lines

Still further provided are engineered neutrophil cell lines from hPSCs. In an embodiment the engineered neutrophil cell line comprises a CAR having the amino acid sequence of SEQ ID NO: 1. In another embodiment the engineered neutrophil cell line comprises a CAR comprising CLTX, the transmembrane domain of CD4, and the intracellular domain of CD3ζ, such as a CAR having the amino acid sequence of SEQ ID NO: 2. In yet another embodiment the engineered neutrophil cell line comprises a CAR having the amino acid sequence of SEQ ID NO: 3.

SEQ ID NOs: 1-3 can be considered reference polypeptide sequences. In other words, some variation (e.g., up to 10%, such as 9%, 8%, 7%, 6%, 5%, 4%, 3%, 2%, or 1%) in the amino acid sequences (e.g., conservative and neutral amino acid substitutions) may be tolerated and achieve similar results. In some instances, some variation in the amino acid sequences may achieve better results. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity and not considering any conservative substitutions as part of the sequence identity. Alignment for purposes of determining percent amino acid sequence identity can be achieved in various ways that are within the skill of the art, for instance, using publicly available computer software. Those skilled in the art can determine appropriate parameters for aligning sequences, including any algorithms needed to achieve maximal alignment over the full length of the sequences being compared.

### Pharmaceutical Compositions

Even still further provided is a pharmaceutical composition. The pharmaceutical composition comprises a population of isolated CAR neutrophils obtained in accordance with an above-described method or a population of neutrophils from an above-described cell line. The pharmaceutical composition further comprises a pharmaceutically acceptable carrier.

The term “isolated” means that the material is removed from its original environment, e.g., the natural environment if it is naturally occurring. For example, a naturally occurring neutrophil present within a living organism is not isolated, but the same neutrophil separated from some or all the coexisting materials in the natural system is isolated.

The term “pharmaceutically acceptable” and grammatical variations thereof, as they refer to compositions, carriers, diluents, reagents, and the like, are used interchangeably and indicate that the materials can be administered to or upon a mammal without undue toxicity, irritation, allergic response, and/or the production of undesirable physiological effects, such as nausea, dizziness, gastric upset, and the like as is commensurate with a reasonable benefit/risk ratio. In other words, it is a material that is not biologically or otherwise undesirable—i.e., the material may be administered to an individual along with CAR neutrophils, for example, without causing any undesirable biological effects or interacting in a significantly deleterious manner with any of the other components of the pharmaceutical composition.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting a composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethanol; (20) phosphate buffered solutions; and (21) other non-toxic, compatible substances employed in pharmaceutical formulations.

The particular formulation employed will depend, at least in part, on the particular route of administration. For example, a formulation suitable for systemic, e.g., intravenous, administration, may differ from a formulation suitable for intracranial administration. Such modifications are within the ordinary skill in the art.

### Methods of Treatment

A method of treating cancer in a subject in need thereof is also provided. The method comprises administering to the subject a therapeutically effective amount of (a) a population of the neutrophils obtained in accordance with an above-described method or a pharmaceutical composition comprising same and a pharmaceutically acceptable carrier, or (b) a population of neutrophils from above-described cell line or a pharmaceutical composition comprising same and a pharmaceutically acceptable carrier.

The cancer can be any cancer. “Cancer” can include, but is not limited to, a group of diseases involving abnormal cell growth with the potential to invade or spread (i.e., metastasize) to other parts of the body. Examples include, but are not limited to, a cancer of the brain, thyroid, lung, pancreas, kidney, stomach, gastrointestinal stroma, endometrium, breast, cervix, ovary, colon, prostate, leukemias, lymphomas, other blood-related cancers, or head and neck cancer. In certain embodiments, the cancer being treated is a tumor. In certain embodiments, the cancer is malignant. The cancer can be a brain tumor. The brain tumor can be a glioma. The glioma can be a glioblastoma. The cancer can express the protein matrix metallopeptidase 2. The population of neutrophils or the pharmaceutical composition comprising same can be administered systemically or intracranially.

The method can reduce, even substantially reduce, systemic and off-target toxicity. By “off-target toxicity” is meant organ or tissue damage or a reduction in the subject's weight that is not desirable to the physician or other individual treating the subject, or any other effect on the subject that is a potential adverse indicator to the treating physician (e.g., B cell aplasia, a fever, a drop in blood pressure, or pulmonary edema).

The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated,” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

The term “patient” or “subject” includes human and non-human animals, such as companion animals (dogs and cats and the like) and livestock animals Livestock animals are animals raised for food production. The subject to be treated is preferably a mammal, in particular a human being.

As used herein, the term “administering” includes all means of introducing the neutrophils, and pharmaceutical compositions comprising same, to the patient. Examples include, but are not limited to, oral (po), parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like. Routes of administration to the brain include, but are not limited to, intraparenchymal, intraventricular, intracranial, and the like.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-known to those skilled in the art.

The term “therapeutically effective amount” as used herein, refers to that amount of engineered neutrophils that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the engineered neutrophils may be decided by the attending physician within the scope of sound medical judgment. The specific therapeutically effective dose level for any particular patient will depend upon a variety of factors, including the disorder being treated and the severity of the disorder; the specific composition employed; the age, body weight, general health, gender and diet of the patient; the time and route of administration; the duration of the treatment; drugs used in combination or coincidentally with the engineered neutrophils; and like factors well-known to the researcher, veterinarian, medical doctor or other clinician of ordinary skill. Thus, the absolute amount of engineered neutrophils included in a given unit dosage form can vary widely, and depends upon factors such as the age, weight and physical condition of the subject, as well as the method of administration.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Administered dosages for the engineered neutrophils for treating cancer, such as a brain tumor, a glioma, a glioblastoma, or a cancer expressing matrix metallopeptidase 2 (MMP2), or other disease or disorder are in accordance with dosages and scheduling regimens practiced by those of skill in the art. Typically, doses >10⁹ cells/patient are administered to patients receiving adoptive cell transfer therapy. Determining an effective amount or dose is well within the capability of those skilled in the art, especially in light of the detailed disclosure provided herein.

The engineered neutrophils administered to a subject can comprise about 1×10⁵ to about 1×10¹⁵ or 1×10⁶ to about 1×10¹⁵ transduced CAR-T cells. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ cells.

The engineered neutrophils administered to a subject can comprise about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, or about 15 million cells. The cells can be administered as a single dose or multiple doses. The engineered neutrophils can be administered in numbers of CAR-expressing neutrophils per kg of subject body weight.

Kits

Finally, a kit for preparing neutrophils from hPSCs is provided. The kit comprises: (a) a glycogen synthase kinase 3 β (GSK3β) inhibitor, optionally as part of a medium; (b) vascular endothelial growth factor (VEGF), optionally as part of a medium; (c) a transforming growth factor β (TGFβ) inhibitor, optionally as part of a medium; (d) granulocyte-macrophage colony-stimulating factor (GM-CSF), interleukin 3 (IL-3), and interleukin 6 (IL-6), optionally as part of a medium; and (e) granulocyte colony-stimulating factor (G-CSF) and a retinoic acid agonist, optionally as part of a medium. The kit can further comprise SCF and Flt3: FMS-like tyrosine kinase 3/fetal liver kinase 2 (Flt3) ligand in (c) and (d) and GlutaMAX and ExCyte in (e).

Examples of GSK3β inhibitor-containing medium include, but are not limited to, DMEM and LaSR basal. Examples of a VEGF-containing medium include, but are not limited to, LaSR basal and Stemline II. Examples of a TGFβ inhibitor-containing medium include, but are not limited to, LaSR basal and Stemline II. Examples of a medium containing GM-CSF, IL-3 and IL-6 include, but are not limited to, Stemline II and StemSpan H3000. Examples of a medium containing G-CSF and AM580 include, but are not limited to, Stemline II and StemSpan H3000. The kit can further comprise SCF and FLT3L (e.g., for use in steps (c) and (d) as set forth above) and/or GlutaMAX and ExCyte (e.g., for use in step (e) as set forth above).

Results

Chemically-Defined Condition Allows Robust Generation of Functional Neutrophils

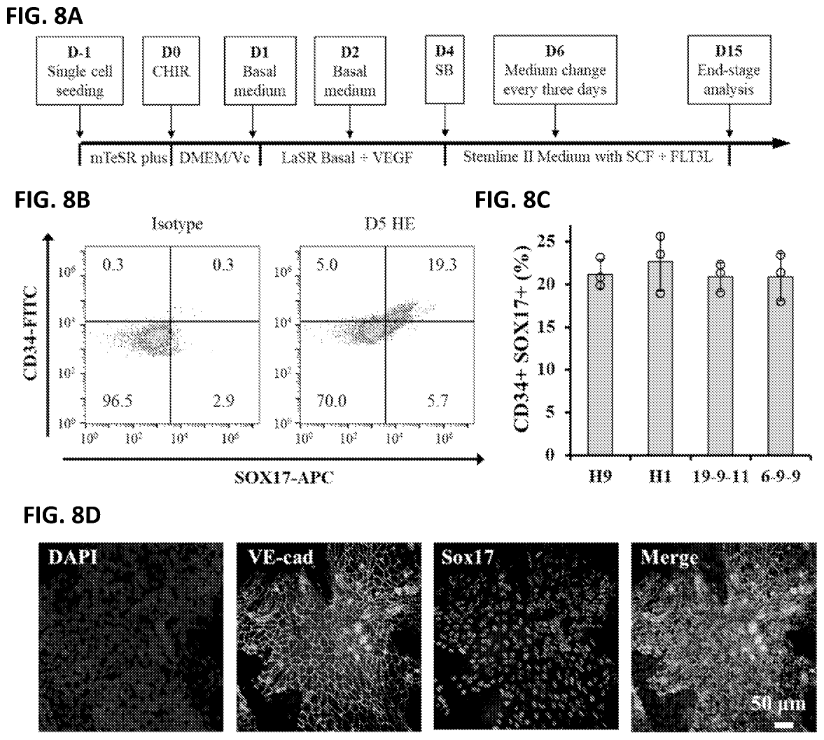

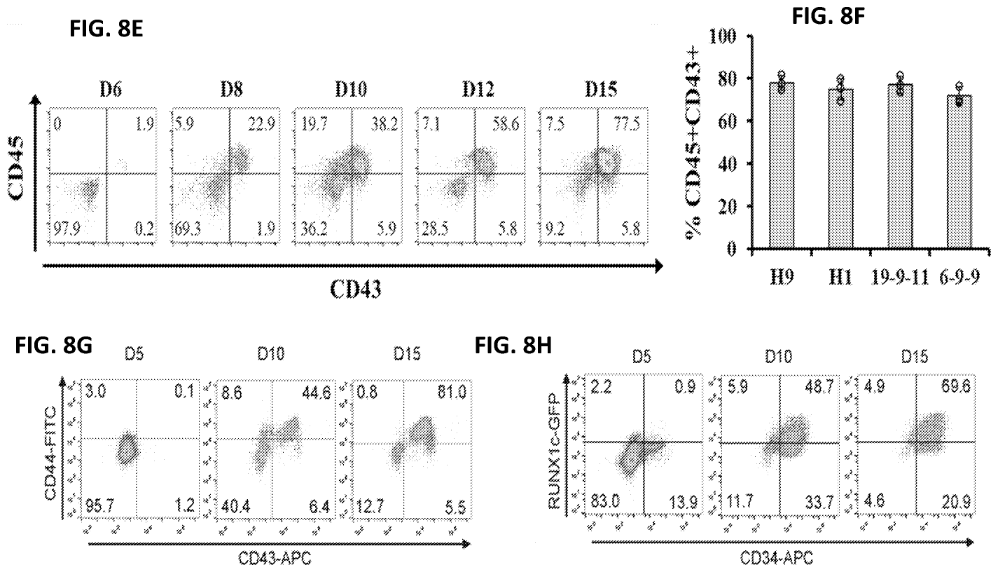

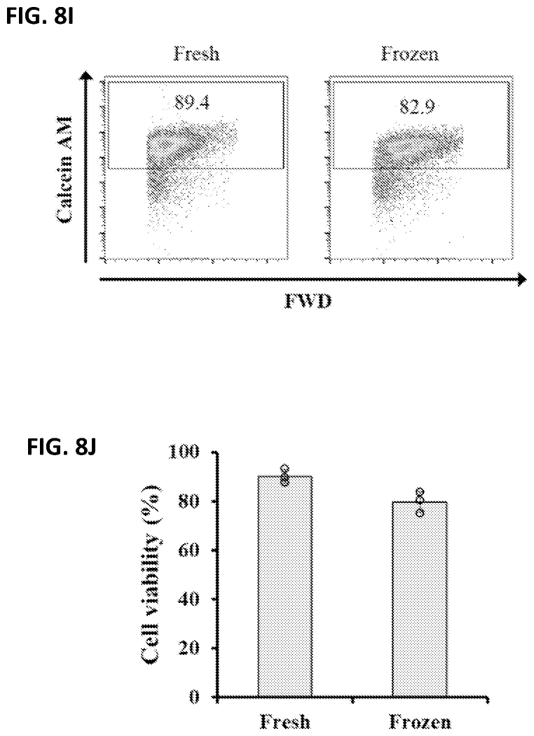

In vitro hematopoietic progenitor induction is the first step to generate neutrophils from hPSCs (Brok-Volchanskaya et al., 2019a; Lachmann et al., 2015; Saeki et al., 2009; Sweeney et al., 2016; Trump et al., 2019). Multipotent hematopoietic stem and progenitor cells (HSPCs) arise from the arterial vasculature in the aorta-gonad-mesonephros (AGM) region through endothelial-to-hematopoietic transition (EHT) (Bertrand et al., 2010; Boisset et al., 2010; Kissa and Herbomel, 2010). We previously induced homogenous CD34+CD31+ hemogenic endothelium (HE) from hPSCs via small-molecule activation of Wnt signaling (FIG. 8A-8B) (Bao et al., 2015; Lian et al., 2014). Importantly, the resulting HE also expressed SOX17 (FIG. 8B-8D), a transcription factor expressed in vascular structures of the AGM and required for HSC generation from AGM (Clarke et al., 2013; Kim et al., 2007; Ng et al., 2016). The employment of TGFβ inhibitor SB431542 (SB) (Bertrand et al., 2010; Boisset et al., 2010; Kissa and Herbomel, 2010) promoted EHT process for the generation of CD45+CD43+ HSPCs (FIG. 8E-8F) that co-expressed definitive hematopoiesis markers CD44 (Fidanza et al., 2019; Oatley et al., 2020) (FIG. 8G) and RUNX1c (Ng et al., 2016) (FIG. 8H). The resulting day 15 hPSC-derived HSPCs also maintained high viability after freeze-thaw (FIG. 8I-8J).

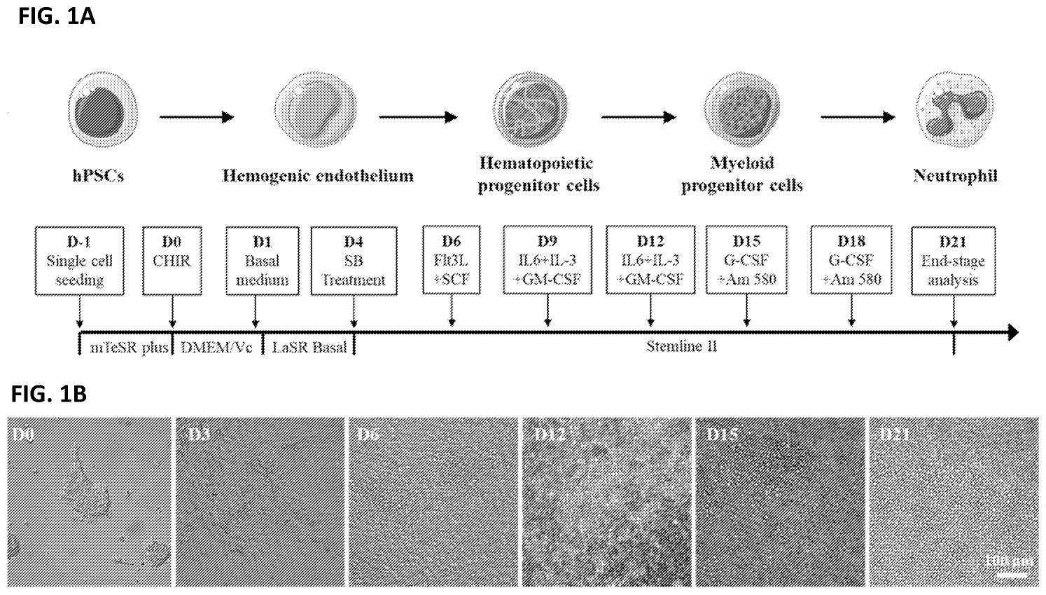

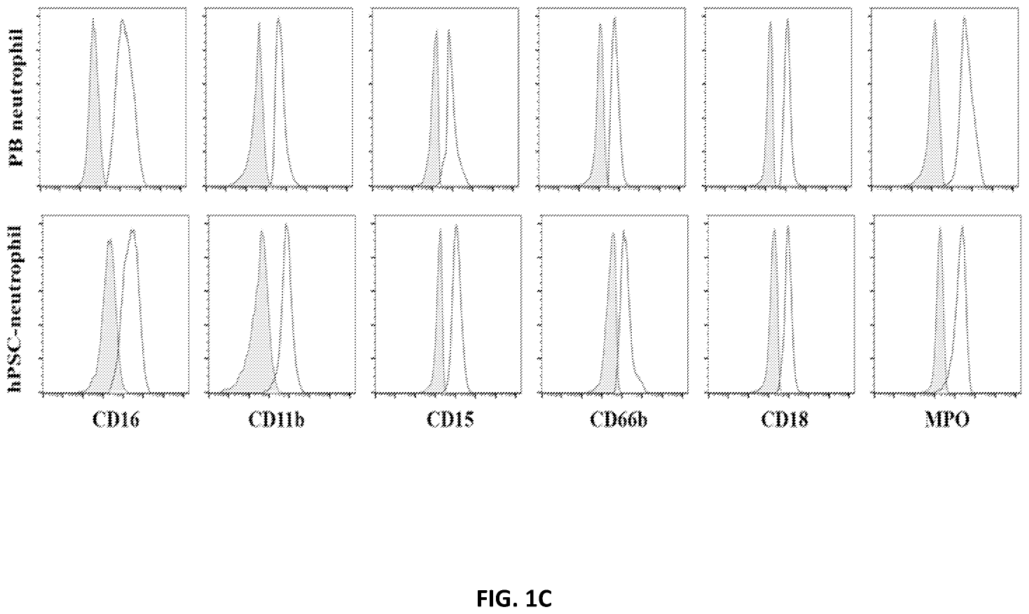

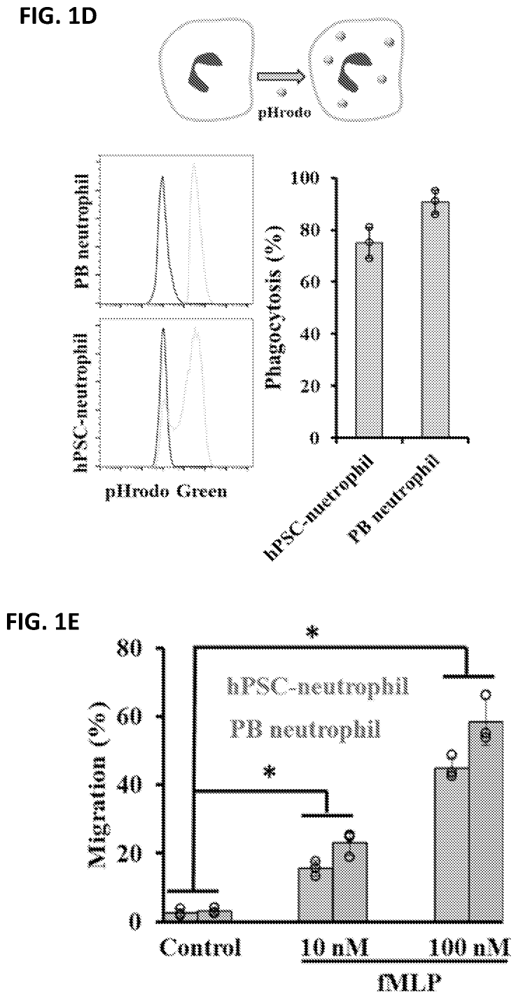

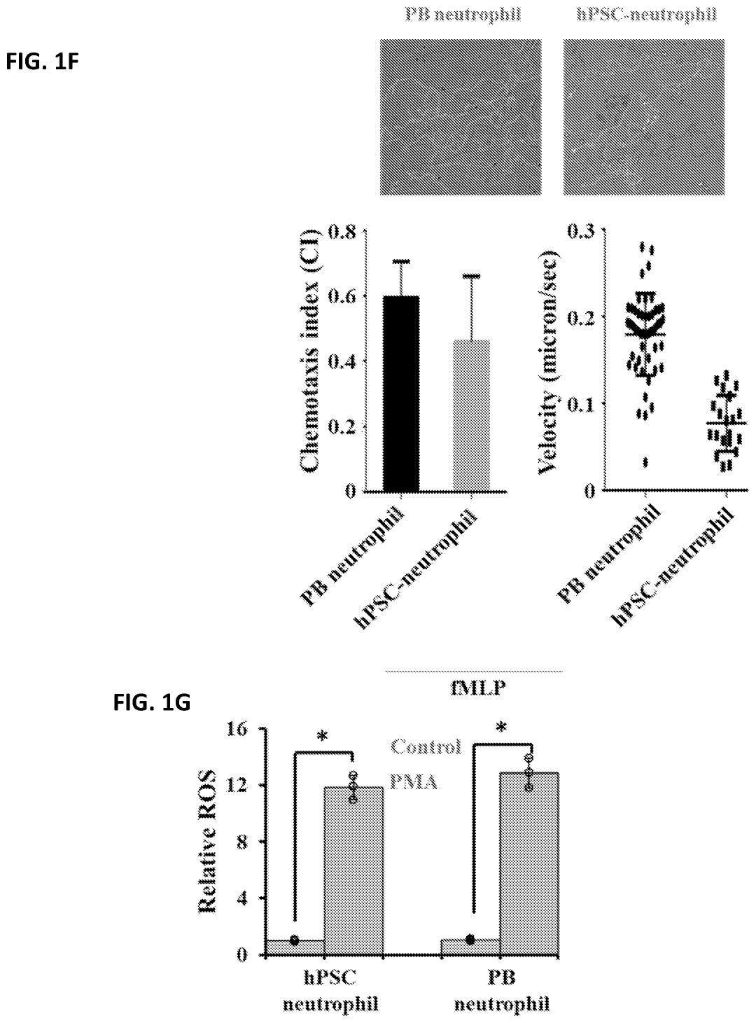

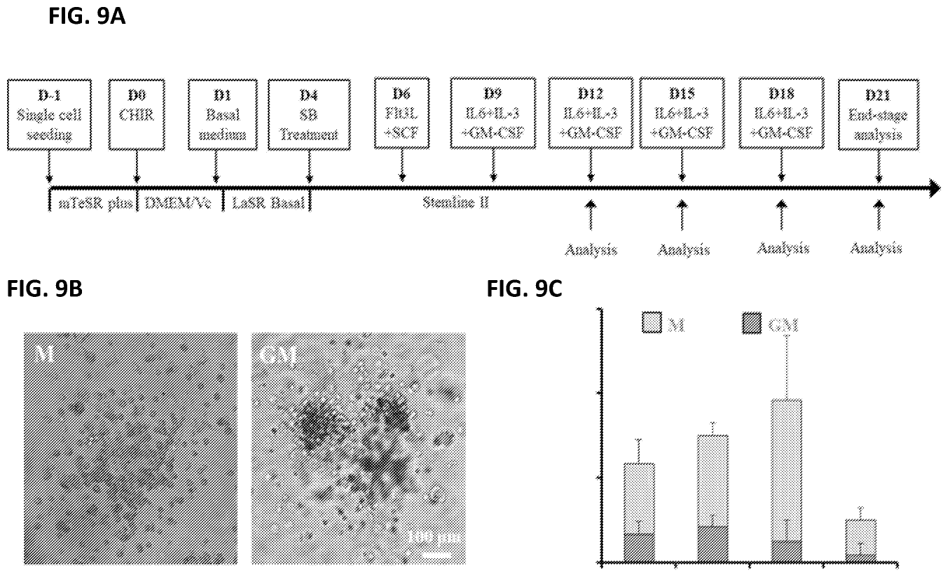

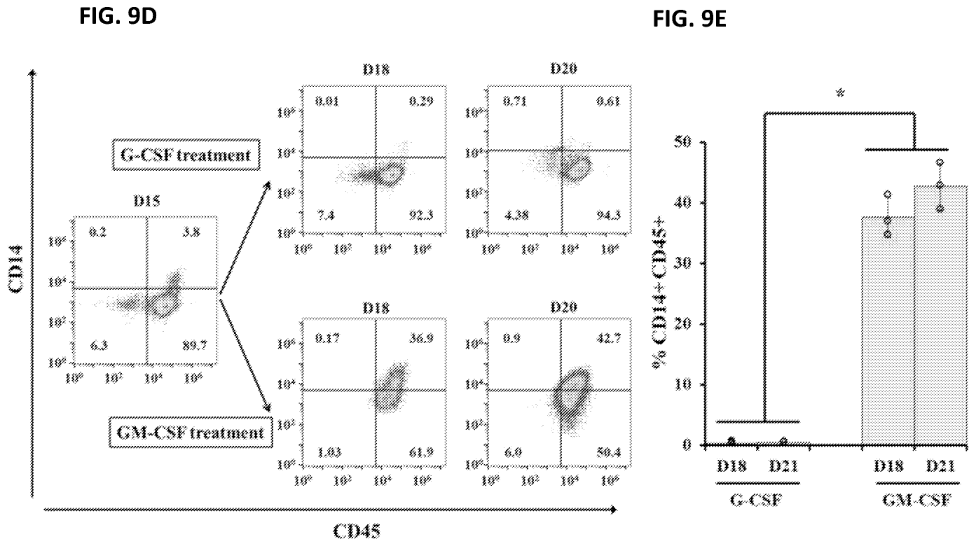

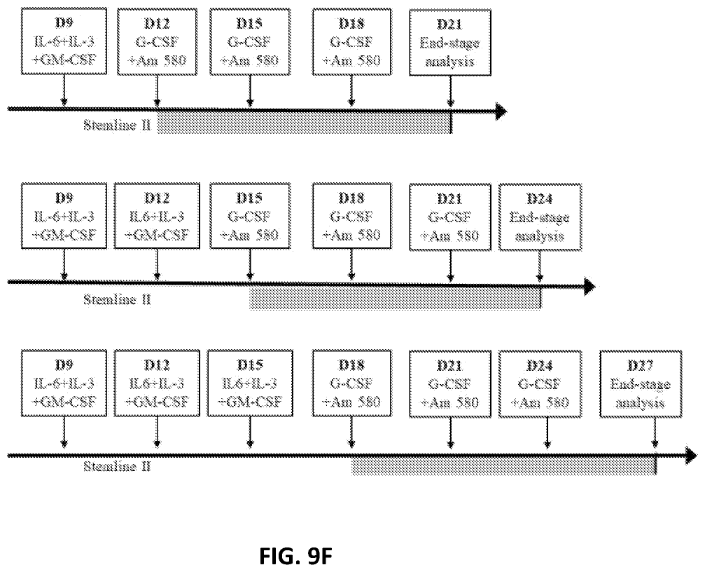

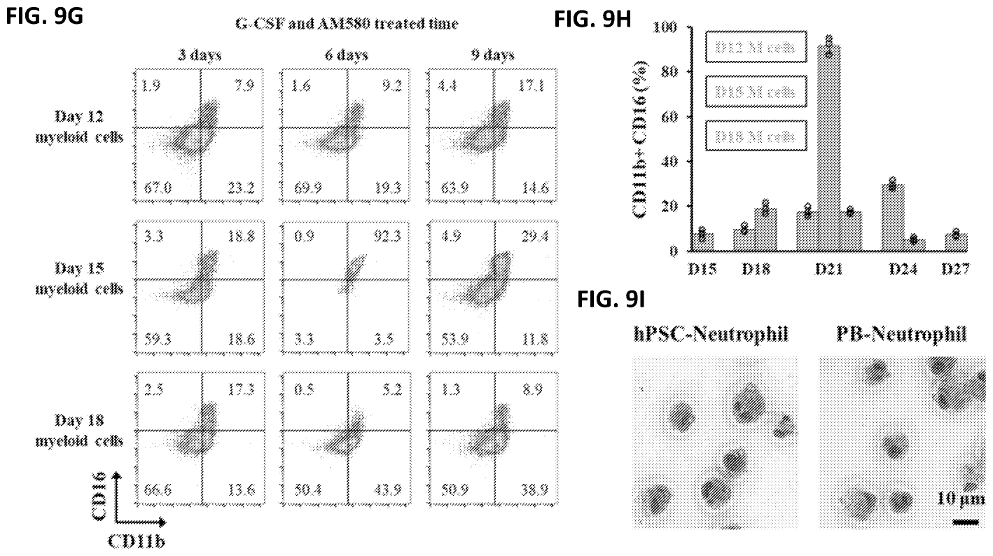

To induce myeloid progenitor and neutrophil differentiation, hPSC-derived HSPC cultures were treated with granulocyte-macrophage colony-stimulating factor (GM-CSF), IL-3, and IL-6 (Cao et al., 2019) from day 9 (FIG. 9A). Floating myeloid progenitor cells collected at different days presented both granulocyte-macrophage (GM) and macrophage (M)-colony-forming potential (FIG. 9B-9C), which increased from day 12 to day 18 and decreased afterwards. To promote neutrophil specification, day 15 myeloid progenitors were treated with granulocyte-CSF (G-CSF). As expected, G-CSF significantly decreased the number of CD14+ monocytes/macrophages as compared to GM-CSF (FIG. 9D-9E). To identify the optimal myeloid progenitor for neutrophil differentiation, floating cells collected at days 12, 15 and 18 were subjected for G-CSF treatment along with AM580 (FIG. 9F), a retinoic acid agonist that promotes neutrophil production from human CD34+ cells (Brok-Volchanskaya et al., 2019b; Li et al., 2016). The efficiency of neutrophil differentiation increased from day 15 to day 21 and significantly decreased afterward, which may be due to the short life-span of neutrophils (FIG. 9G-9H). We also identified day 15 myeloid progenitors with 6-day treatment of G-CSF and AM580 as the optimal condition for neutrophil differentiation, which was thus employed in our subsequent experiments (FIG. 1A). Dynamic morphological changes along with the emergence of hematopoietic clusters from day 12 were observed (FIG. 1B). The resulting day 21 neutrophils displayed a typical neutrophil morphology (FIG. 9I) and manifested high expression levels of neutrophil-specific markers (FIG. 1C), including CD16, CD11b, CD15, CD66b, CD18 and MPO, as compared to their counterparts isolated from human peripheral blood (PB).

To evaluate the function of hPSC-derived neutrophils, we performed phagocytosis and chemotaxis assays. Similar to primary PB neutrophils, hPSC-derived neutrophils effectively phagocytosed pHrodo E. coli bioparticles (FIG. 1D) and displayed excellent transmigration ability in chemotaxis models using transwells (FIG. 1E) and microfluidics (FIG. 1F) (Afonso et al., 2013). We also measured the production of reactive oxygen species (ROS) from hPSC-derived neutrophils. In response to the phorbol 12-myristate 13-acetate (PMA), hPSC-derived neutrophils generated comparable ROS to PB neutrophils (FIG. 1G). Collectively, we established a novel chemically-defined, feeder-free platform for robust production of functional neutrophils from hPSCs with a yield of ˜20 neutrophils per hPSC, thus highlighting its potential applications in studying neutrophil biology and treating neutropenia.

AAVS1 Targeted CAR Knockin Improves Anti-Tumor Cytotoxicity of hPSC-Derived Neutrophils

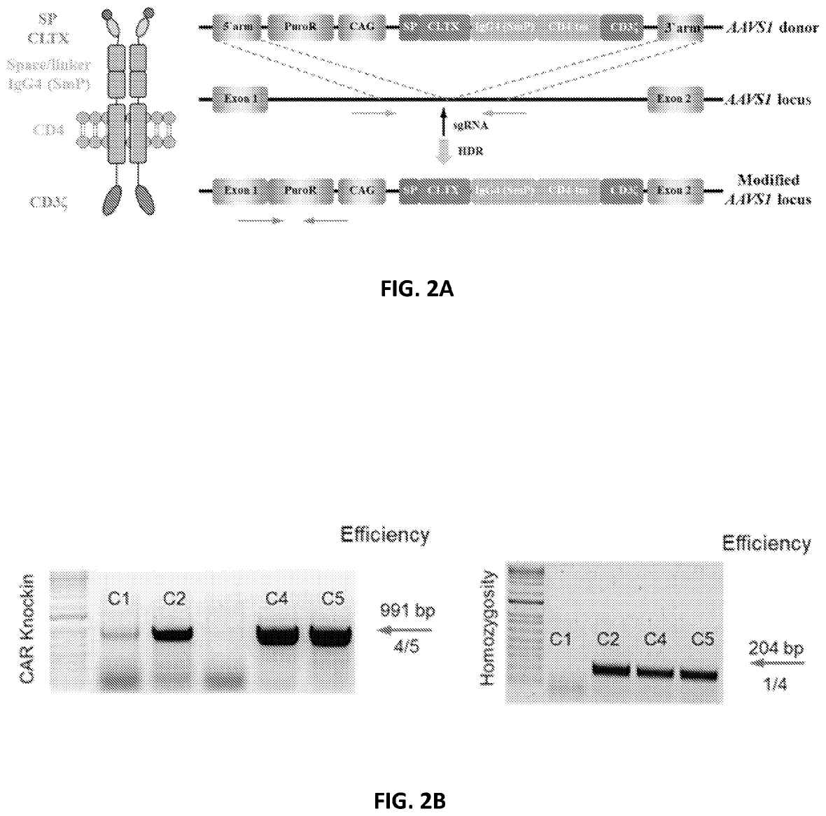

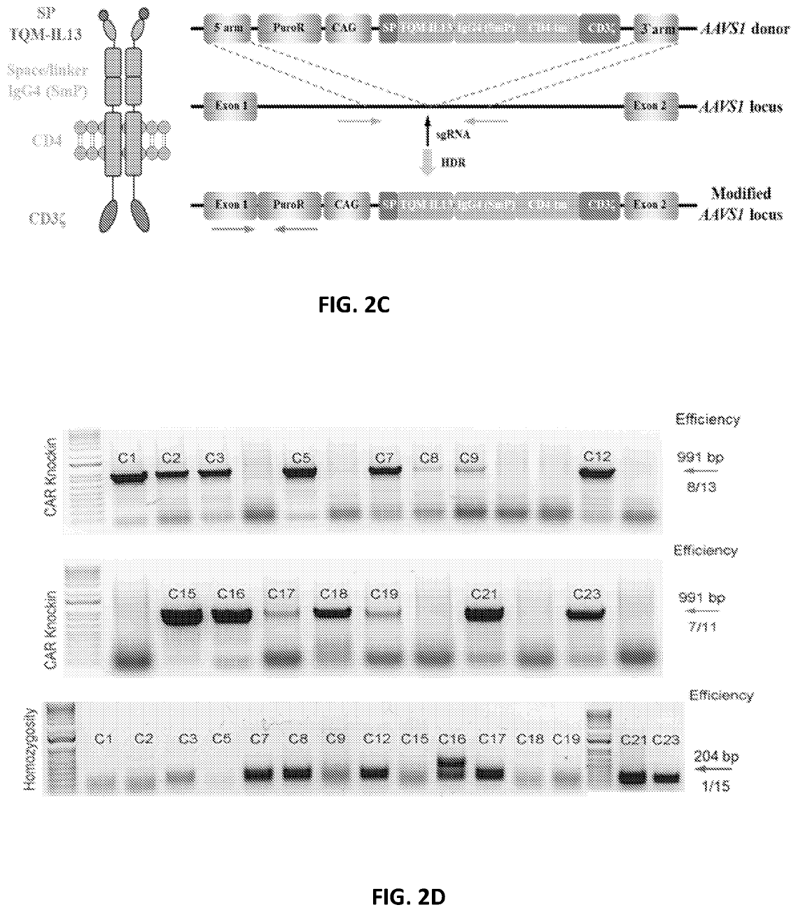

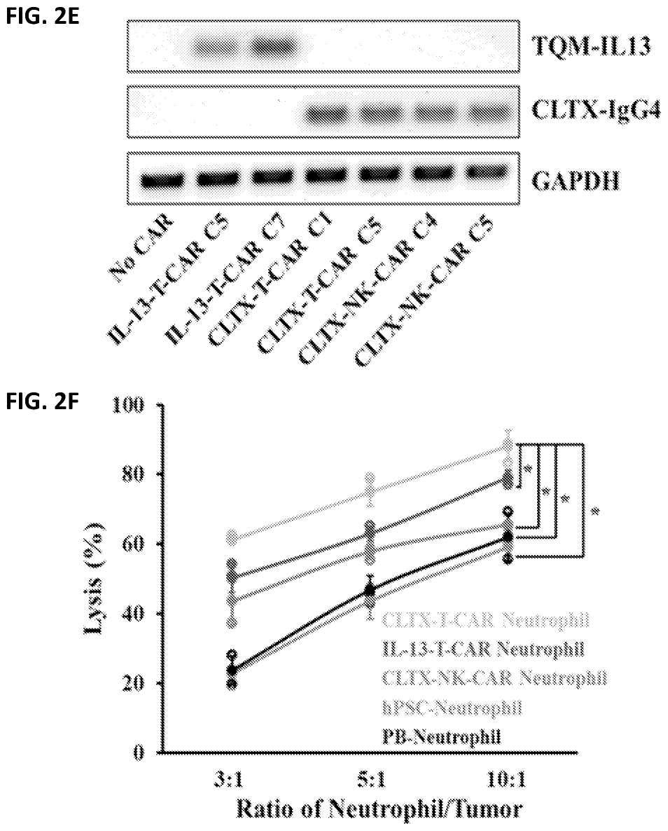

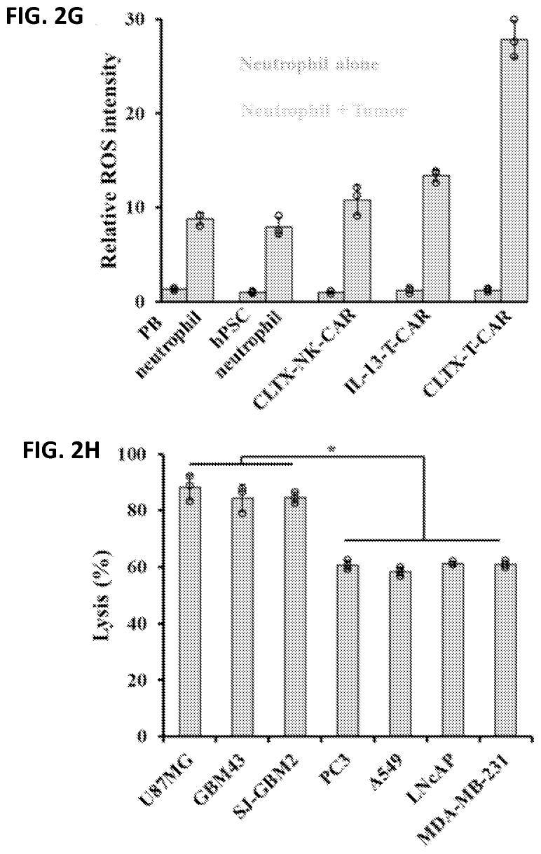

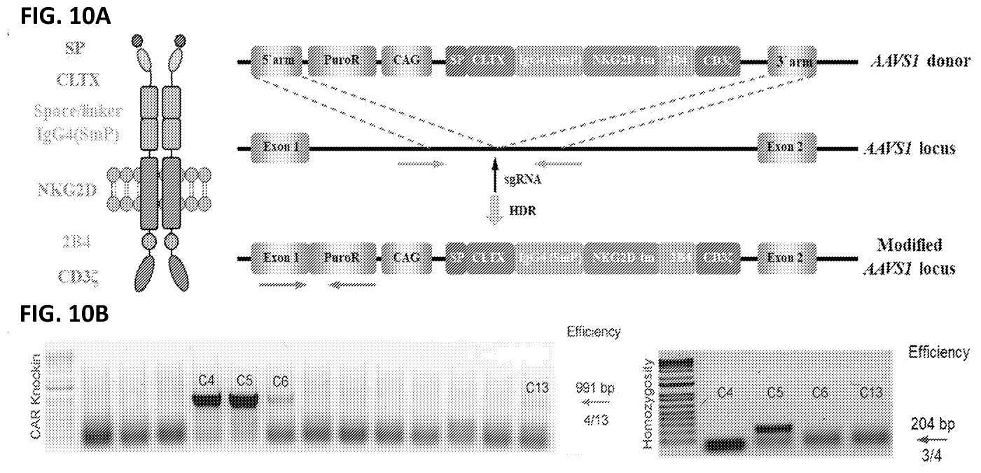

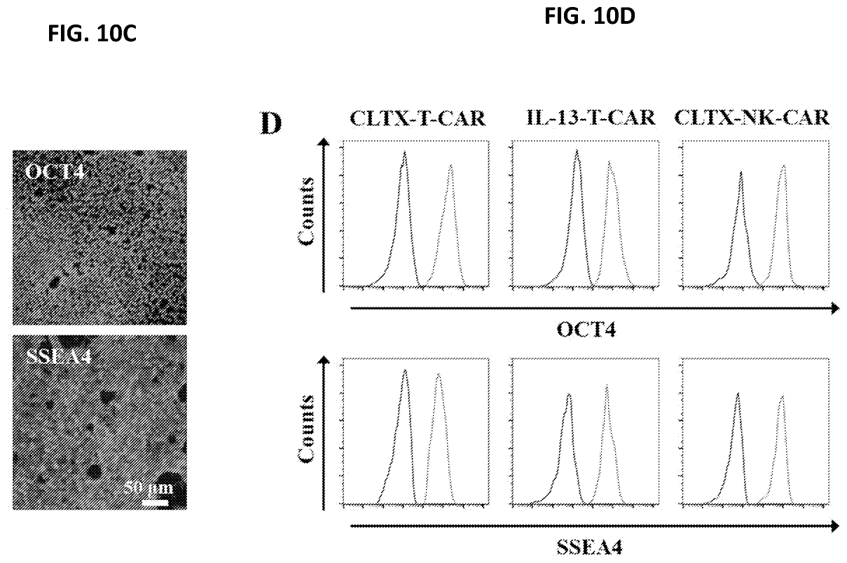

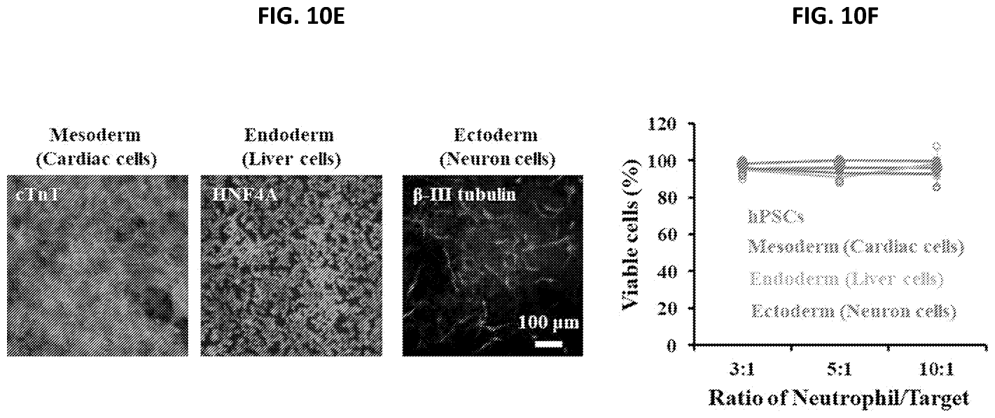

Primary neutrophils from healthy donors present potent cancer-killing activity against various human cancer cell lines in vitro (Yan et al., 2014). Thus, we sought to determine whether de novo neutrophils derived from hPSCs are able to directly kill tumor cells, and whether chimeric antigen receptor (CAR) expression could enhance their anti-tumor cytotoxicity for targeted immunotherapy. To achieve stable and uniform expression of CARs on neutrophils, we directly knocked CAR constructs into the endogenous AAVS1 safe harbor locus of H9 hPSCs, a widely-used site for constitutive transgene expression in human cells (Smith et al., 2008), via CRISPR/Cas9-mediated homologous recombination (FIGS. 2A-2H, FIGS. 10A-10F). Three different CAR constructs were designed using T or natural killer (NK) cell-specific transmembrane and intracellular domains: CLTX-T-CAR (FIG. 2A-B), IL-13-T-CAR (FIG. 2C-2D), and CLTX-NK-CAR (FIG. 10A-10B). After nucleofection, puromycin-resistant (PuroR) single cell-derived hPSC clones were isolated and subjected for PCR genotyping. Approximately 60% (3 out of 5), 58.3% (14 out of 24), and 7.7% (1 out of 13) of the clones were targeted in one allele (heterozygous), and approximately 20% (1 out of 5), 4.2% (1 out of 24) and 23.1% (3 out of 13) in both alleles (homozygous) for CLTX-T-CAR (FIG. 2A-B), IL-13-T-CAR (FIG. 2C-2D), and CLTX-NK-CAR (FIG. 10A-10B), respectively. The stable expression of synthetic CARs on the genetically-modified hPSCs were confirmed by RT-PCR analysis of CLTX-IgG4 and IL-13 fragments (FIG. 2E). Importantly, CAR-expressing hPSCs retained high expression levels of pluripotent markers SSEA-4 and OCT-4 (FIG. 10C-10D).

To determine the effects of CAR expression on the anti-tumor cytotoxicity of neutrophils, CAR-expressing hPSCs were differentiated into CAR-neutrophils (FIG. 1A), which were then co-cultured with glioblastoma (GBM) U87MG cells in vitro at different effector-to-target ratios. As compared to wildtype or other CAR-expressing neutrophils, CLTX-T-CAR hPSC-neutrophils displayed superior tumor-killing activities (FIG. 2F). Neutrophils could also release cytotoxic ROS to kill target cells (Yan et al., 2014), and the kinetics of ROS production in different neutrophils coincided well with their increased tumor killing abilities (FIG. 2G), indicating the potential involvement of ROS in neutrophil-mediated cytotoxicity against GBM cells. In addition, enhanced anti-tumor cytotoxicity was only observed in the co-incubation of CLTX-T-CAR hPSC-neutrophils and GBM cells, including U87MG cell line, primary adult GBM43, and pediatric SJ-GBM2 cells (FIG. 2H), suggesting their high specificity towards GBM. Importantly, CLTX-T-CAR neutrophils did not kill normal hPSCs or hPSC-derived cells (FIG. 10E-10F), consistent with a previous report that primary neutrophils do not kill healthy epithelial cells (Yan et al., 2014). Collectively, hPSC-derived CAR-neutrophils presented enhanced anti-tumor cytotoxicity against target cancer cells and produced more ROS in vitro, highlighting their potential in targeted immunotherapy.

CLTX-T-CAR hPSC-Neutrophils Kill Glioblastoma Cells by Trogoptosis

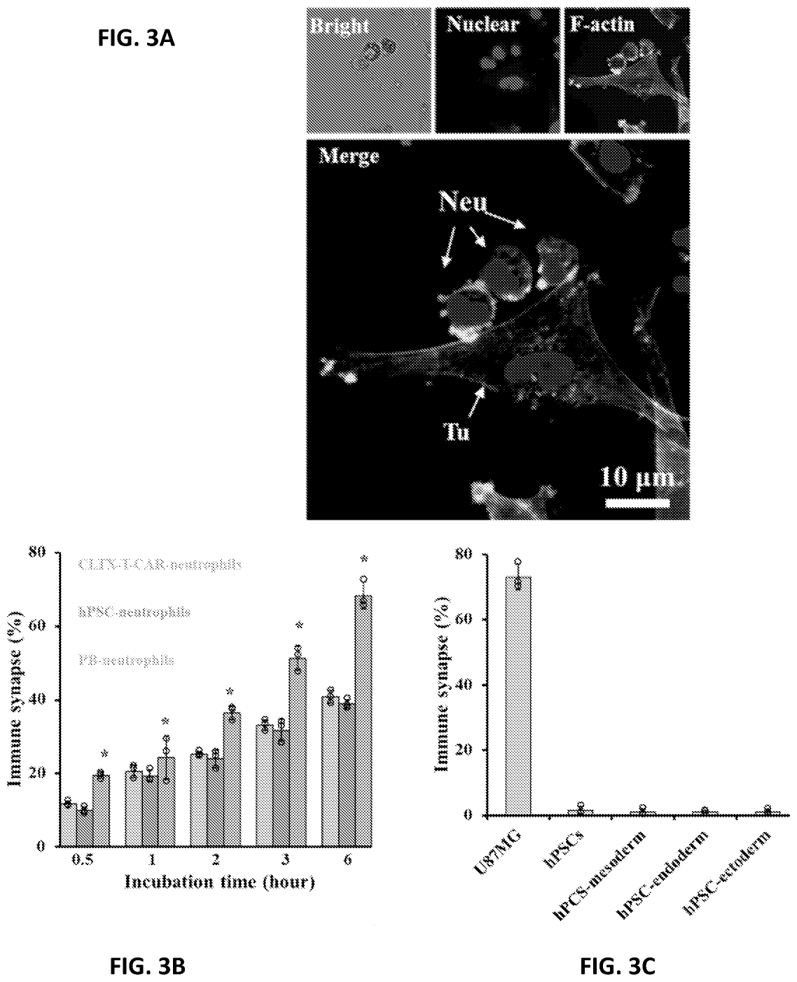

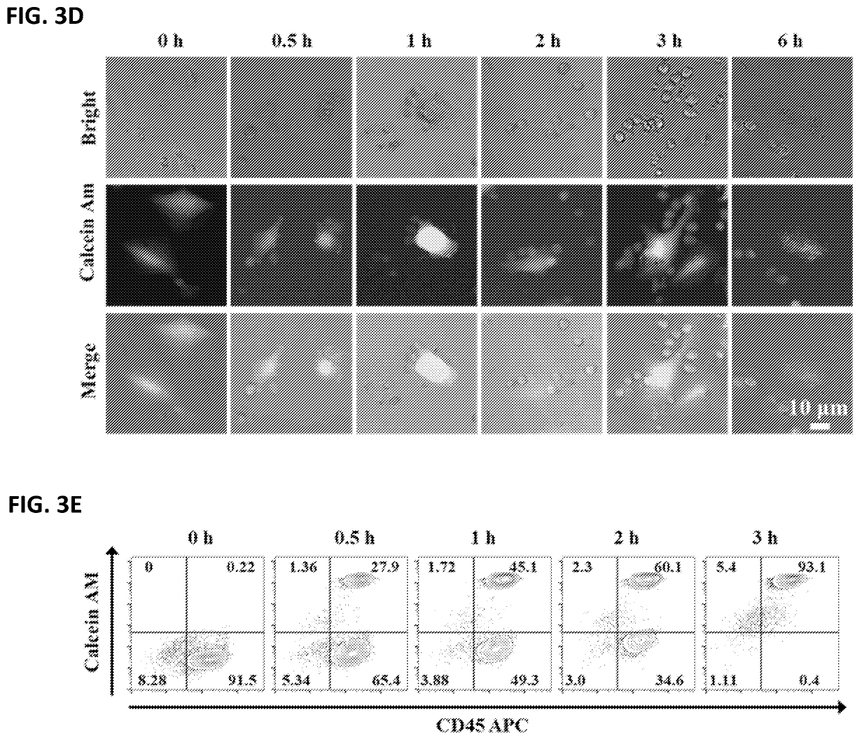

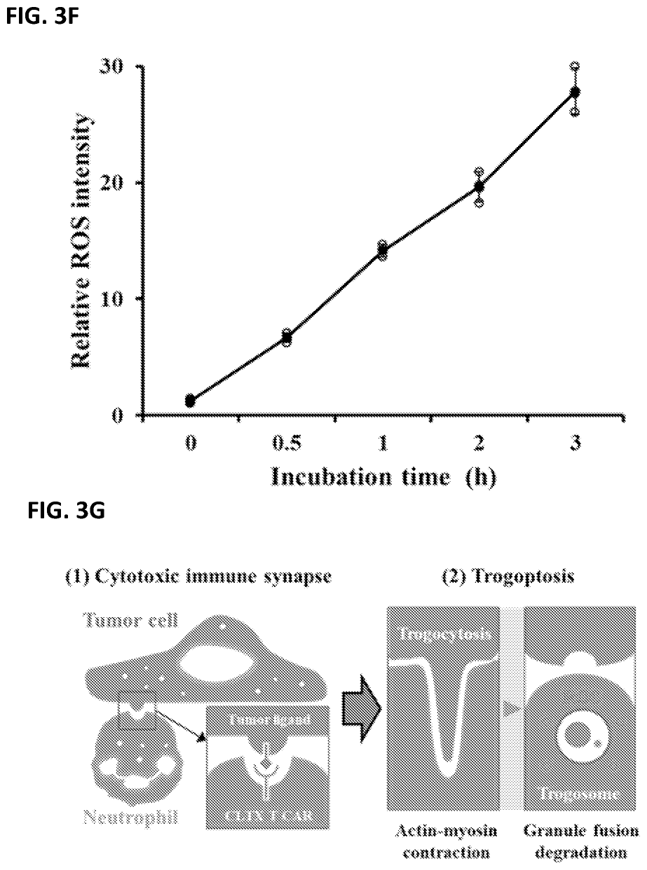

To explore underlying mechanisms of CAR-neutrophil-mediated anti-tumor cytotoxicity, direct effector-target interactions were investigated since intimate neutrophil-tumor conjugate formation was required for cytotoxicity by neutrophils (Matlung et al., 2018) Immunological synapses between neutrophils and targeted tumor cells formed after half-an-hour co-culture and increased proportionally with incubation time (FIG. 3A-3B). As expected, more effector-target interactions were observed between CLTX-T-CAR hPSC-neutrophils and tumor cells as compared to primary PB- and hPSC-neutrophils (FIG. 3B), whereas no immunological synapses formed between CAR-neutrophils and normal hPSCs or hPSC-derived cells (FIG. 3C), highlighting their specificity against the tumor cells. Live cell imaging revealed that CAR-neutrophils actively migrating toward tumor cells and disruption of target cell plasma membrane, as indicated by the disrupted cell appearance and neutrophil uptake of pre-loaded cytosolic dye Calcein-AM as early as half an hour following co-incubation (FIG. 3D-3E). Neutrophils phagocytosed tumor cell membrane fragments after conjugating with target cells and induced tumor cell death (FIG. 3D-3E), consistent with previously reported neutrophil trogoptosis, a trogocytosis-related tumor necrosis involved in the antibody-dependent cellular cytotoxicity (ADCC) of neutrophils (Matlung et al., 2018). Furthermore, the dynamics of ROS release agreed well with the kinetics of neutrophil trogocytosis (FIG. 3F), suggesting a prominent role of ROS and trogocytosis in neutrophil-mediated tumor cell killing. Collectively, our results demonstrated that hPSC-derived neutrophils could kill the tumor cells via trogoptosis and ROS release after forming immunological synapses with target cells (FIG. 3G), consistent with previous reports (Matlung et al., 2018; Yan et al., 2014).

CLTX-T-CAR hPSC-Neutrophils Specifically Bind to Glioblastoma Via MMP2

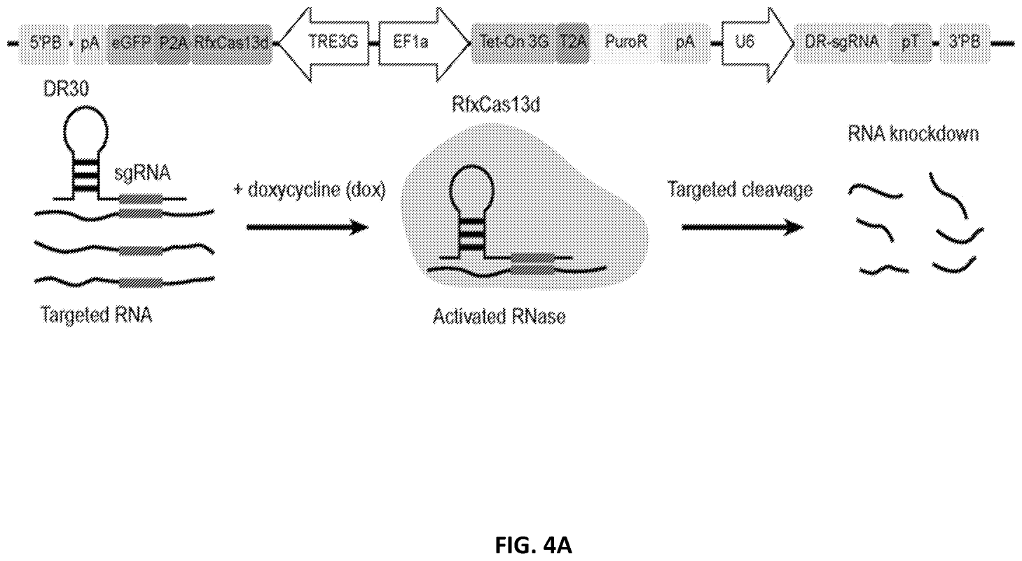

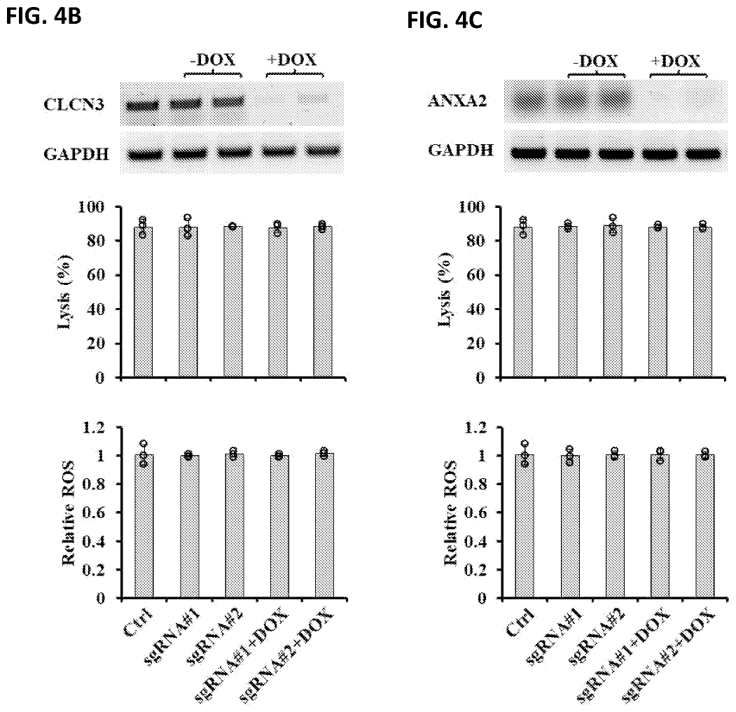

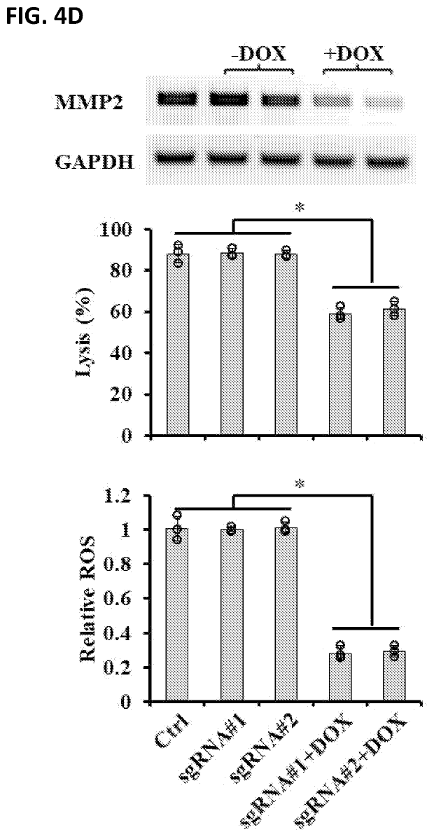

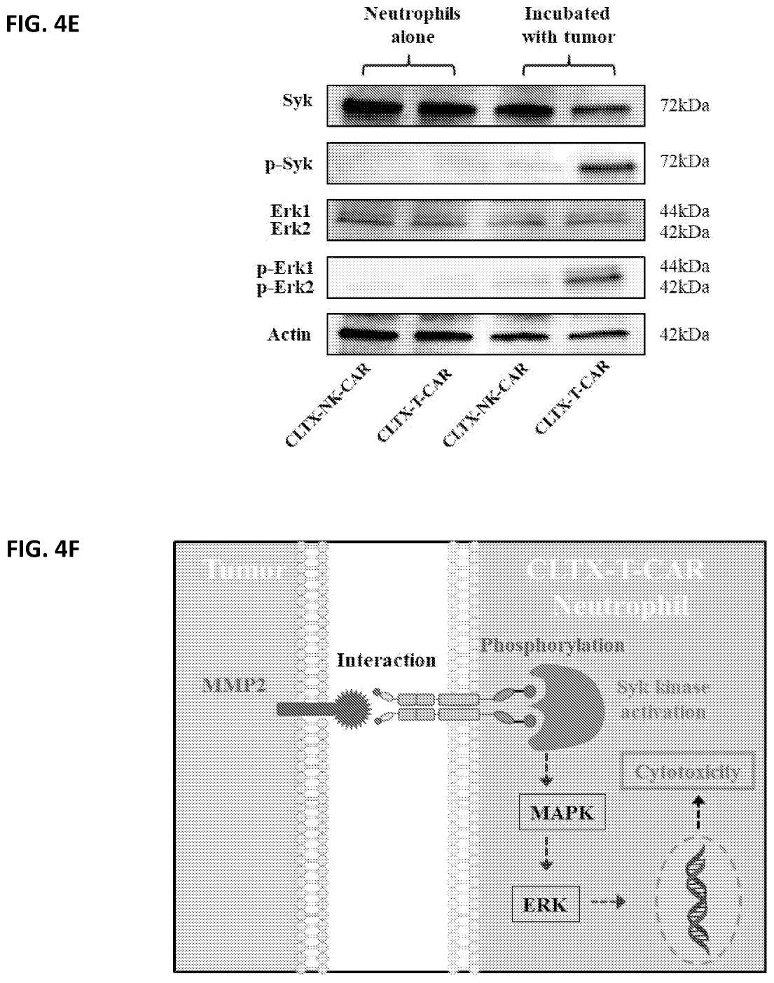

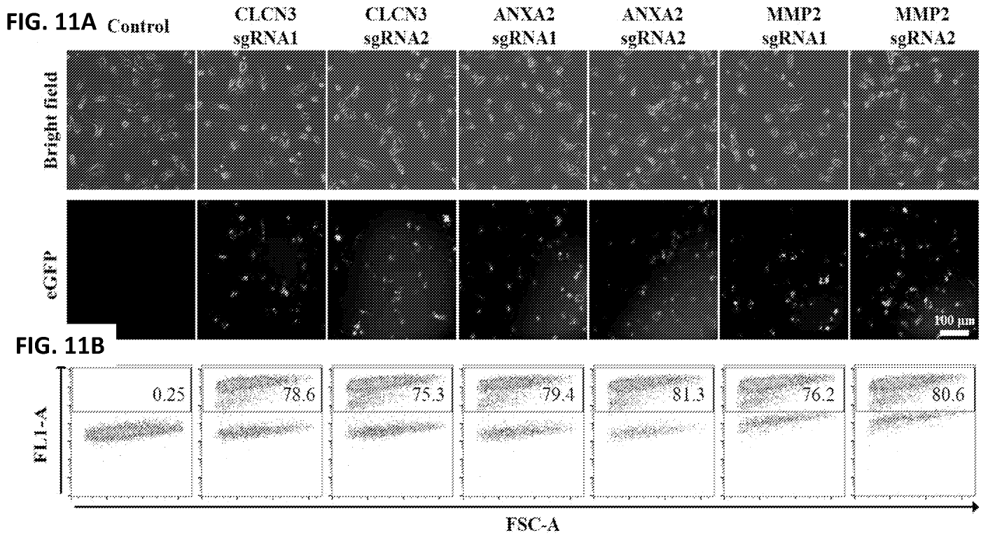

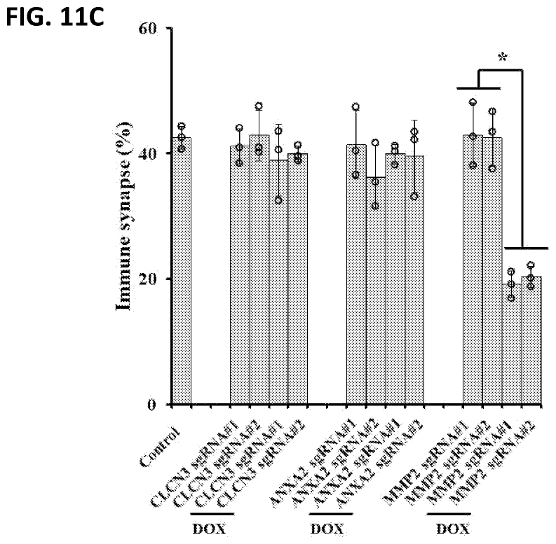

To further explore molecular mechanisms underlying CLTX-T-CAR-enhanced cytotoxicity against tumor cells, we implemented an all-in-one inducible Cas13d-mediated gene knockdown platform (FIG. 4A) to determine the membrane protein associated with CLTX binding, including chloride channels (CLCN3), phospholipid protein annexin A2 (ANXA2), and matrix metalloproteinase 2 (MMP2) (Wang et al., 2020a). After puromycin selection, approximately 78% of the transfected glioblastoma cells expressed Cas13d as indicated by the eGFP expression (FIG. 11A-11B) in the presence of doxycycline (DOX). RT-PCR analysis confirmed the successful knockdown of CLCN3 (FIG. 4B), ANXA2 (FIG. 4C), and MMP2 (FIG. 4D) in U87MG GBM cells. Notably, knockdown of MMP2, but not CLCN3 or ANXA2, significantly reduced CLTX-T-CAR neutrophil-mediated tumor cell killing and ROS production (FIG. 4B-4D). Substantially decreased formation of immunological synapses between CAR-neutrophils and MMP2 defective tumor cells was also observed (FIG. 11C). These findings demonstrate that membrane protein MMP2 is required for CLTX-T-CAR recognition and activation of neutrophil to kill tumor cells. This suggests the safety of CLTX-T-CAR neutrophils in future clinical application, given the negligible MMP2 expression on human normal tissues (Itoh, 2015).

We next investigated downstream intracellular signaling in activated neutrophils after binding to MMP2-expressing tumor cells. Primary neutrophils display antibody-dependent cellular cytotoxicity (ADCC) toward tumor cells via trogoptosis, which is mediated by Fcγ receptor and its downstream signaling pathways, including tyrosine kinase Syk (Matlung et al., 2018). CLTX-T-CAR hPSC-neutrophils displayed stronger phosphorylated activation of Syk (p-Syk) upon GBM stimulation, as compared to their counterparts with CLTX-NK-CAR (FIG. 4E). Notably, significantly increased ratio of extracellular signal-regulated kinase (Erk) 1/2 (p-Erk1/2), a key signaling mediator involved in lymphoid-mediated cytotoxicity (Li et al., 2018), was also observed in our GBM-stimulated CLTX-T-CAR hPSC-neutrophils. This indicates potential activation of Syk-vav1-Erk pathway in activated neutrophils (FIG. 4F), reminiscent of intracellular signaling transduction in CAR hPSC-NK cells (Li et al., 2018).

CLTX-T-CAR hPSC-Neutrophils Display High Transmigration and Anti-Tumor Cytotoxicity Activities in Biomimetic Tumor Models In Vitro

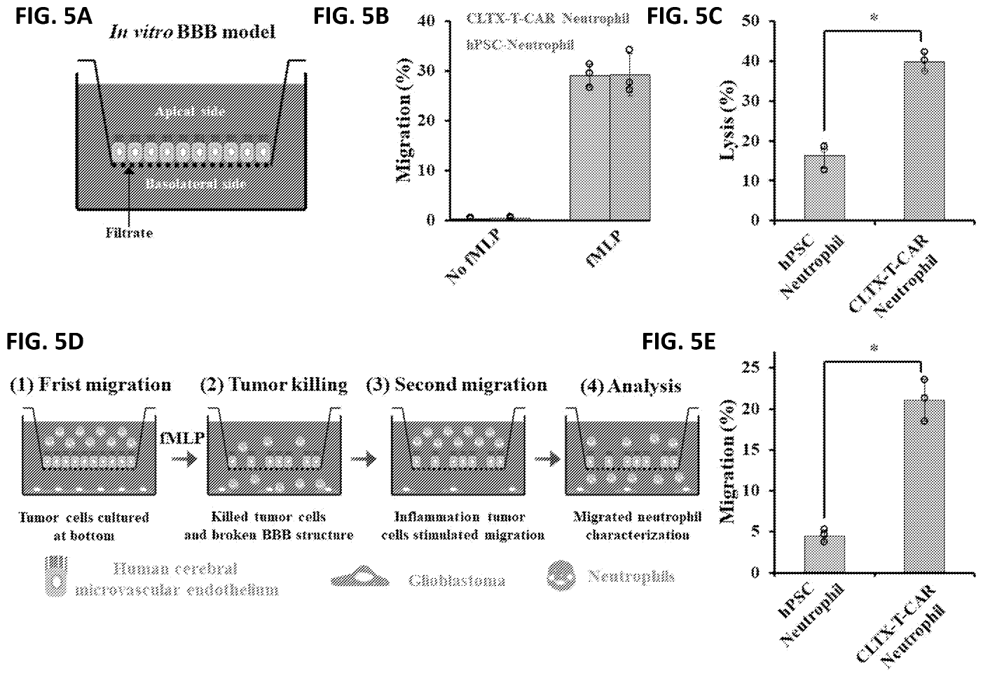

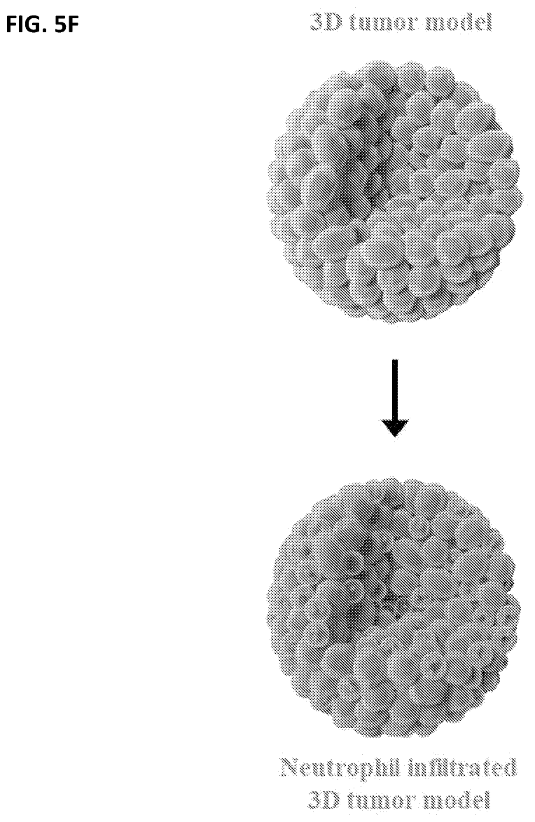

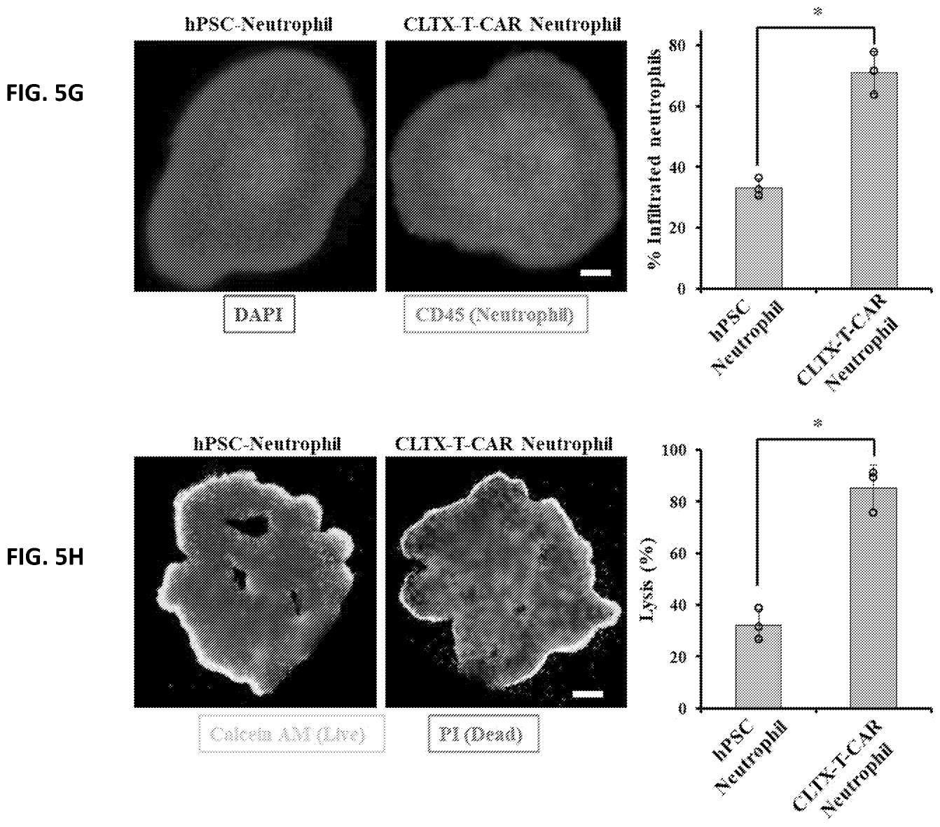

To further evaluate the activities of CAR-neutrophils, we implemented a transwell-based blood brain barrier (BBB) model (FIG. 5A) using human cerebral microvascular endothelial cells. While CAR-expressing and wildtype hPSC-neutrophils displayed similar transmigration activity across the BBB in response to N-Formylmethionine-leucyl-phenylalanine (fMLP) (FIG. 5B), CAR-neutrophils demonstrated higher tumor killing ability after migration (FIG. 5C). Furthermore, CLTX-T-CAR hPSC-neutrophils retained high transmigration ability during their second trafficking across the BBB in response to the inflammatory tumor cells (FIG. 5D-5E), recapitulating many aspects of in vivo inflammation and cancer. A simple three-dimensional (3D) GBM model was also implemented to construct an in vivo tumor niche-like microenvironment that contains a dense extracellular matrix network and heterogeneous tumor cell subtypes (FIG. 5F). As compared to the wildtype control, CLTX-T-CAR hPSC-neutrophils exhibited higher tumor-infiltrating (FIG. 5G) and tumor-killing (FIG. 5H) activities in the 3D tumor model. Taken together, CLTX-T-CAR hPSC-neutrophils retained high transmigration ability and anti-tumor cytotoxicity under tumor-niche mimicking conditions, highlighting their potential application in targeted immunotherapy.

CLTX-T-CAR hPSC-Neutrophils Display Enhanced Activity Against Glioblastoma In Vivo

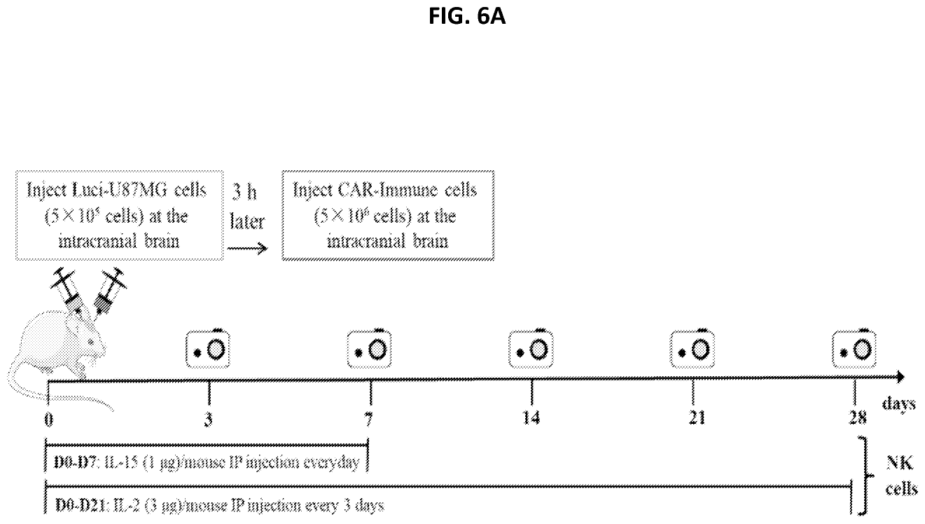

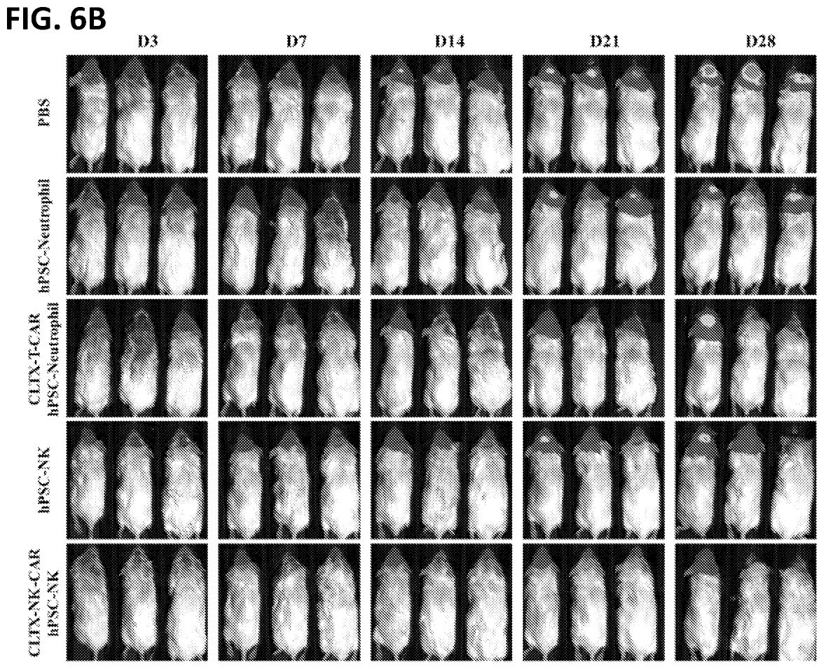

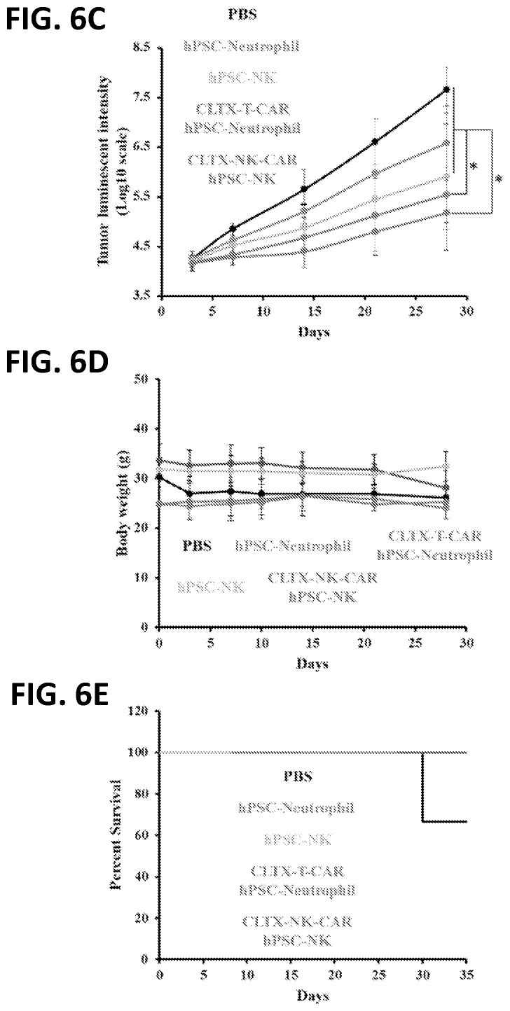

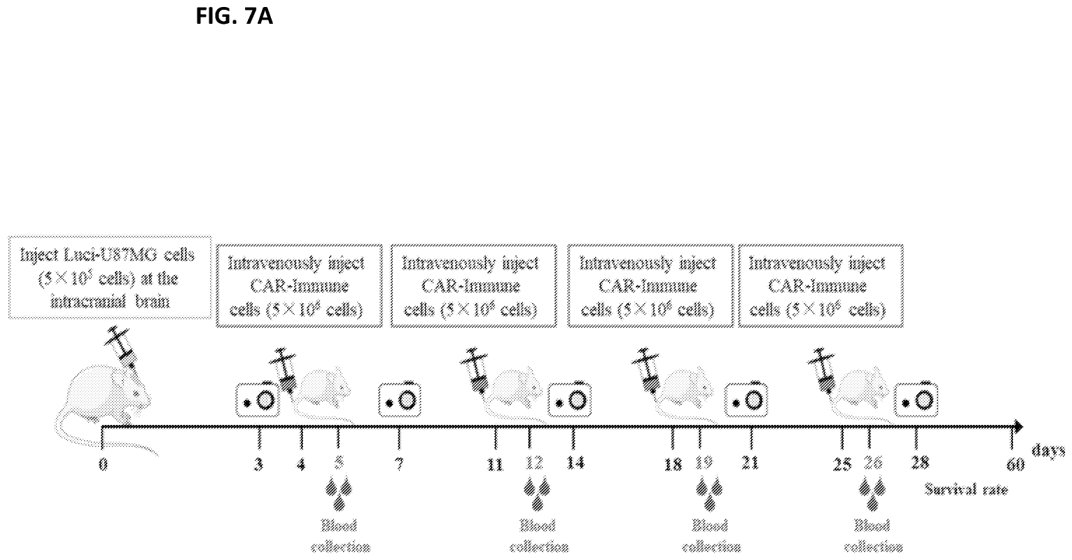

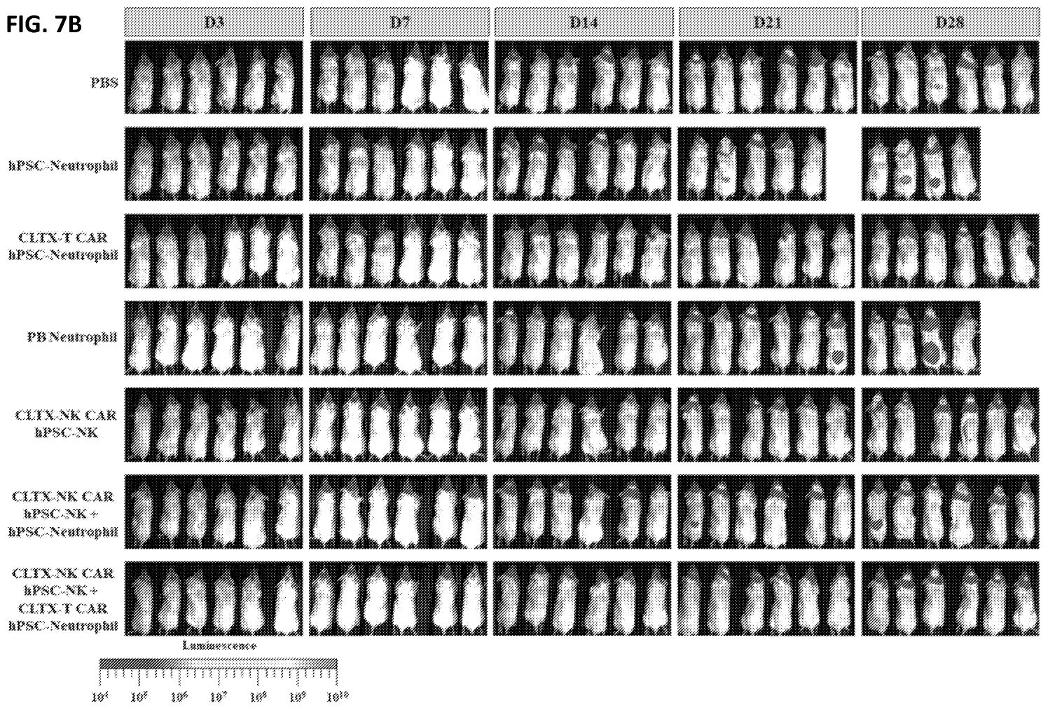

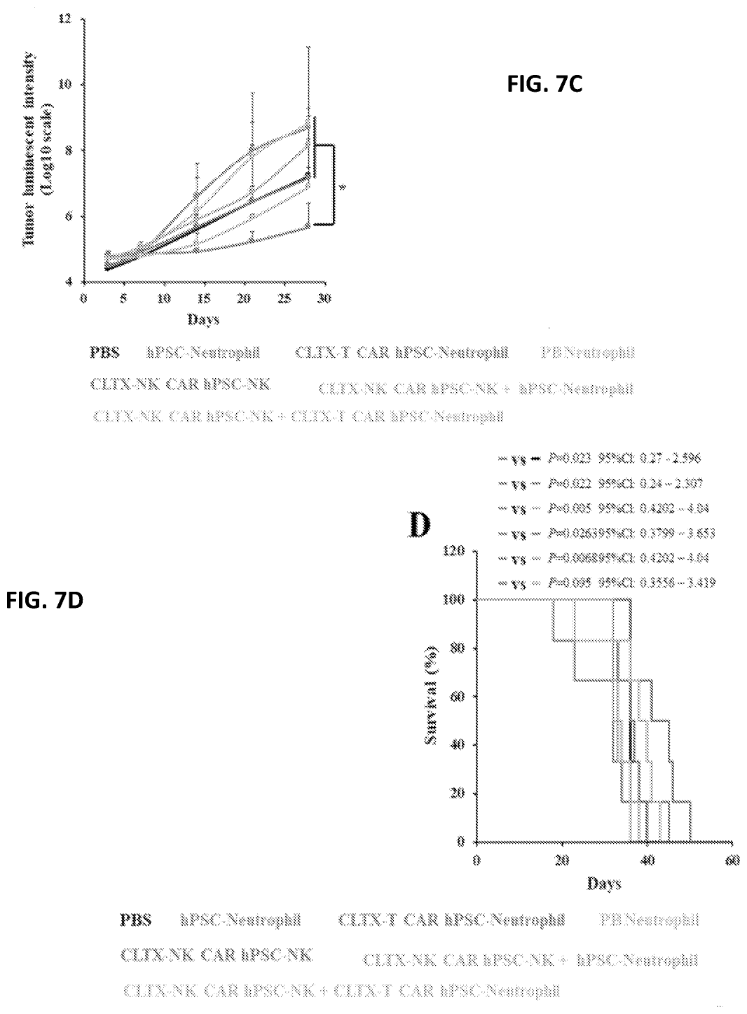

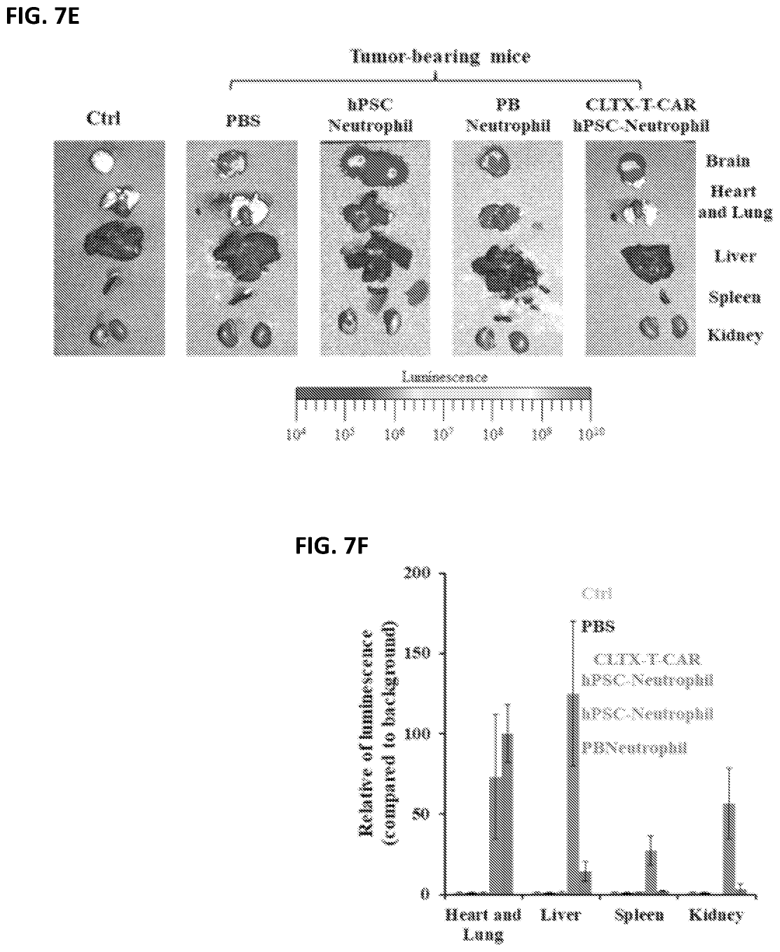

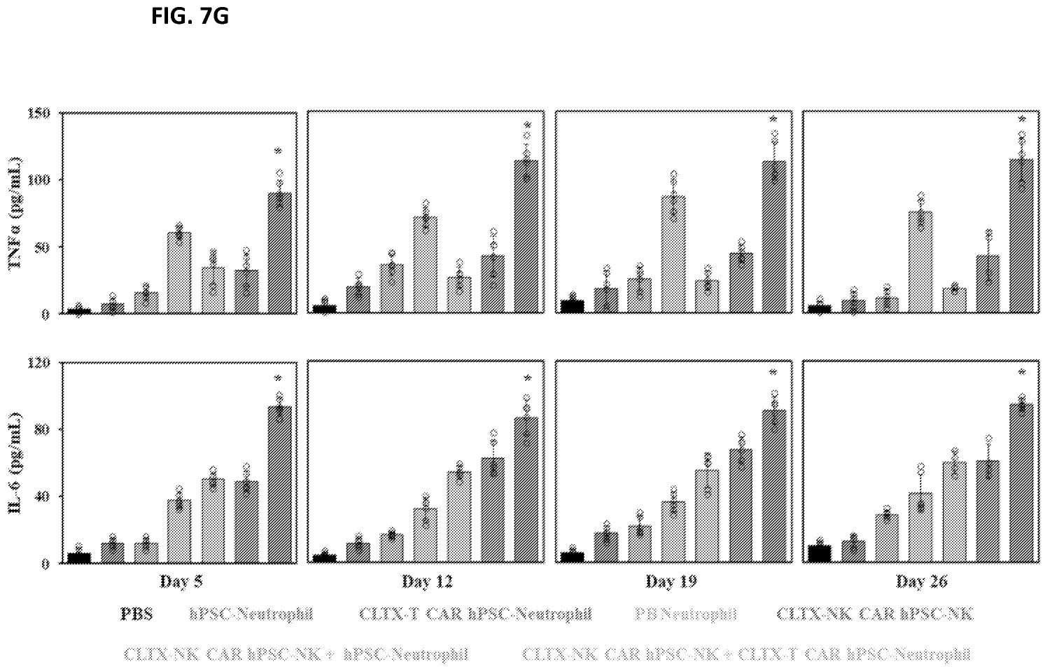

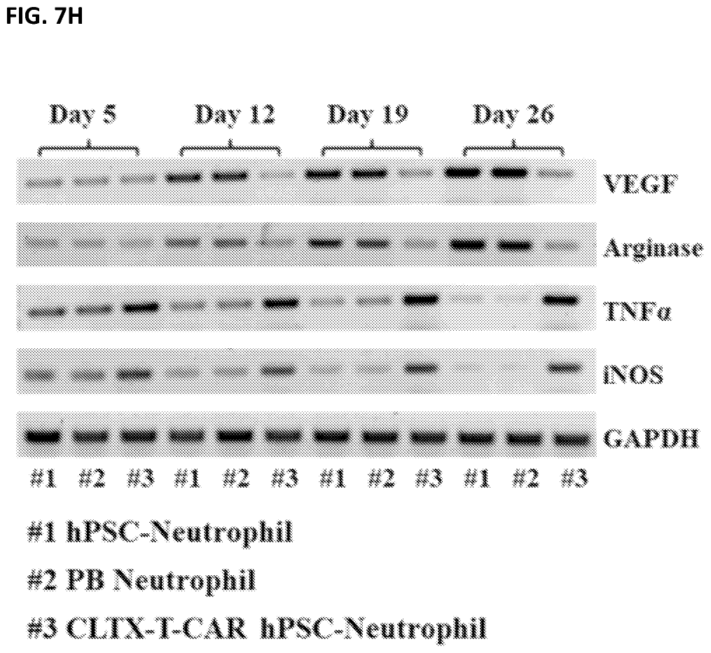

To determine the function of CLTX-T-CAR hPSC-neutrophils in vivo, we implemented an in situ xenograft model via intracranial injection of 5×10⁵ luciferase-expressing GBM cells into the brain of immunodeficient mice (Wang et al., 2020b). Neutrophils were administrated intratumorally (FIGS. 6A-6E) or intravenously (FIGS. 7A-7H) to investigate their in vivo tumor-killing activities. In the intratumoral injection experiment, tumor-bearing mice were intratumorally administrated with a single dose of PBS, 5×10⁶ wildtype or CLTX-T-CAR hPSC-neutrophils or hPSC-NK cells 3 hour following tumor cell inoculation (FIG. 6A). Bioluminescent imaging (BLI) was performed to monitor tumor growth weekly after initial imaging on day 3 (FIG. 6B). As compared to PBS-treated mice, treatment with neutrophils or NK cells significantly reduced tumor burden (FIG. 6B-6C). As expected, CLTX-T-CAR hPSC-NK cells and neutrophils displayed higher anti-tumor cytotoxicity than the wildtype controls in the mice that maintained a stable body weight (FIG. 6D). Notably, one of the PBS-treated tumor-bearing mice died at day 30 due to the overgrowth of tumor in the recipient brain (FIG. 6B, 6E).

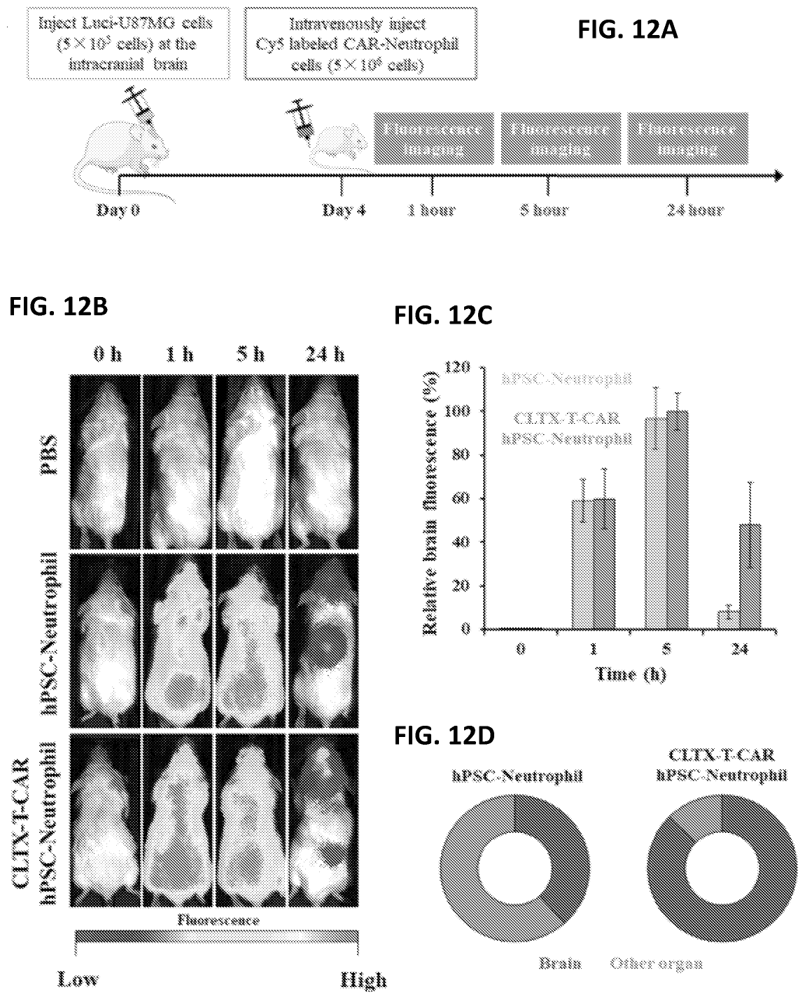

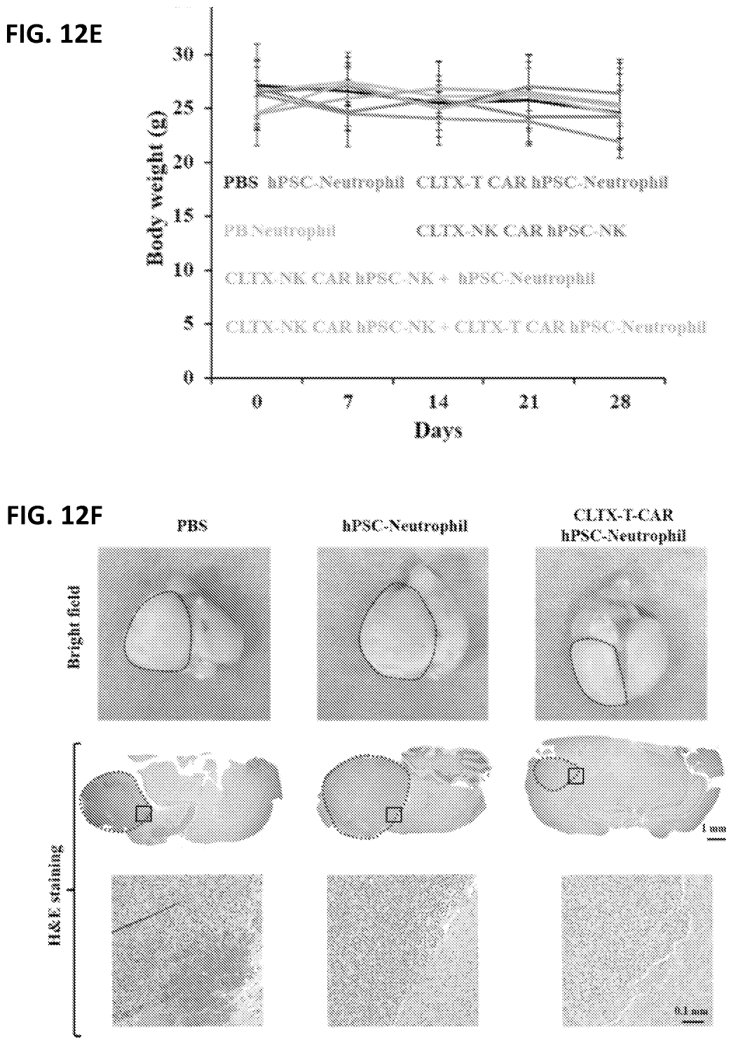

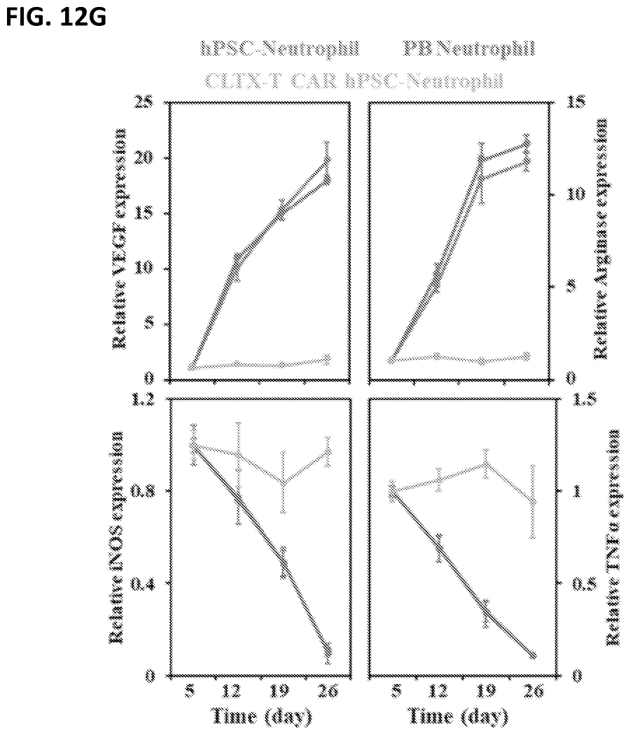

We next investigated in vivo activities of CAR-neutrophils via weekly intravenous administration of 5×106 neutrophils into tumor-bearing mice (FIG. 7A). To track in vivo biodistribution and trafficking of CAR-neutrophils, we labeled neutrophils with Cy5 before systemic injection and performed fluorescence imaging 1, 5 and 24 hours after systemic administration (FIG. 12A). Neutrophils trafficked to the whole mouse body in an hour and retained a similar biodistribution in 5 hours after neutrophil injection (FIG. 12B). Compared to wildtype neutrophils, CAR-neutrophils effectively crossed BBB and trafficked to GBM xenograft in the mouse brain after 24 hours (FIG. 12C-12D). No significant changes of body weight were observed across the experimental mouse groups during the intravenous administration study (FIG. 12E). Consistent with the intratumoral administration study, CAR-neutrophils displayed higher anti-tumor cytotoxicity than PBS and wildtype controls in mice according to BLI analysis (FIG. 7B-7C) as well as brightfield and H&E staining images of GBM xenografts (FIG. 12F).

Notably, tumor-bearing mice treated with CLTX-T-CAR hPSC-neutrophils demonstrated a significantly reduced tumor burden as compared to those treated with CAR-NK cells, suggesting the superior ability of neutrophils in crossing BBB and penetrating GBM xenograft in mice. In contrast to CAR-neutrophils, weekly administration of wildtype hPSC-neutrophils or peripheral blood (PB) neutrophils significantly promoted the growth of tumor in the brain with or without CARNK cells, and resulted in the death of tumor-bearing mice as early as day 21 (FIG. 7D). Despite the rarity of extraneural metastasis of GBM (˜200 reported cases (Rosen et al., 2018)) and unknown pathogenesis (Seo et al., 2012), systemic metastasis occurred in the tumor-bearing mice treated with wildtype hPSC-derived or PB-neutrophils as determined by BLI images of various ex vivo organs and/or tissues (FIG. 7E-7F), suggesting a potential role of neutrophils in the extracranial metastasis of human patient GBM (Liang et al., 2014; Wang et al., 2020c).

We next measured human cytokine production release in plasma of different experimental mouse groups, including TNF α and IL-6. All non-PBS experimental groups produced detectable TNF α and IL-6 in plasma from day 5 to day 26, and CAR-neutrophils maintained highest levels of both cytokines (FIG. 7G). To further explore underlying mechanism of neutrophil-mediated metastasis, we harvested human neutrophils from mouse blood and subjected for anti-tumor (N1) or pro-tumor (N2) phenotype analysis (Fridlender et al., 2009; Shaul et al., 2016). Tumor xenografts significantly decreased expression of N1-specific markers, including iNOS and TNFα, and increased N2-specific markers, including VEGF and Arginase (Shaul et al., 2016), in wildtype hPSC- or PB-neutrophils (FIG. 7H, FIG. 12G). On the contrary, CLTX-T-CAR hPSC-neutrophils retained high expression levels of N1 markers, which is consistent with their strong anti-tumor cytotoxicity and cytokine release in tumor-bearing mice. Collectively, our findings clearly demonstrated that hPSC-derived CAR-neutrophils can sustain an anti-tumor phenotype and efficiently kill tumor cells under various tumor niche-like conditions tested in this study, highlighting their potential application in targeted immunotherapy.

Maintenance and Differentiation of hPSCs

H9 was obtained from WiCell and maintained on Matrigel- or iMatrix 511-coated plates in mTeSR plus medium according to a previously published method. For differentiation, hPSCs were dissociated with 1 mM EDTA and seeded onto iMatrix 511-coated, 24-well plates at a cell density between 10,000 and 80,000 cells/cm² in mTeSR plus medium with 5 μM Y27632 for 24 hours (day −1). At day 0, cells were treated with 6 μM CHIR99021 (CHIR) in DMEM medium supplemented with 100 μg/mL ascorbic acid (DMEM/Vc), followed by a medium change with LasR basal medium at day 1, day 2, and day 3 and the addition of 50 ng/mL VEGF to the medium from day 2 to day 4. At day 4, medium was replaced by Stemline II medium (Sigma) supplemented with 10 μM SB431542. After 2 days, SB431542-containing medium was aspirated, and cells were maintained in Stemline II medium with 50 ng/mL SCF and FLT3L. At day 9 and day 12, the medium was aspirated and changed to Stemline II medium containing 50 ng/mL SCF and FLT3L and 25 ng/mL GM-CSF. At day 15, floating cells were gently harvested and used for terminal neutrophil differentiation. The floating cells were cultured in Stemline II medium supplemented with GlutaMAX 100X, ExCyte (0.2%), human G-CSF (150 ng/mL), and Am580 retinoic acid agonist (2.5 μM). After three days, the same medium with all components and cytokines was added to the top of the existing culture. Mature neutrophils were gently harvested from the supernatant after five days of culture.

Separately, homogeneous CD34+CD31+ hemogenic endothelium was induced from hPSCs via small molecule activation of Wnt signaling, as shown in FIGS. 8A-8C. FIG. 8A is a schematic diagram showing an overview of the differentiation of neutrophils from human induced pluripotent stem cells (iPSCs). FIG. 8B shows flow cytometry analysis of hPSC-derived day 5 cultures for CD34/SOX8 expression. The resulting hemogenic endothelium expressed SOX17, which is a transcription factor expressed in vascular structure of the AGM and required for human stem cell generation from AGM. FIG. 8C is a bar graph of H9 human embryonic stem cells (hESCs), H1 hESCs, 19-9-11 induced pluripotent stem cells (iPSCs), and 6-9-9 induced iPSCs vs. CD34+SOX17+(%).

The addition of the TGFβ inhibitor SB431542 promoted endothelial-to-hematopoietic transition, resulting in the generation of CD45+CD43+ HSPCs, which co-expressed definitive hematopoiesis markers CD44 and RUNX1c, as shown in FIGS. 8D-8H. FIG. 8A is a schematic diagram showing an overview of the differentiation of neutrophils from human induced pluripotent stem cells (iPSCs). FIG. 8B shows flow cytometry analysis of hPSC-derived day 5 cultures for CD34/SOX17 expression. FIG. 8C is a bar graph of H9 human embryonic stem cells (hESCs), H1 hESCs, 19-9-11 induced pluripotent stem cells (iPSCs), and 6-9-9 induced iPSCs vs. CD34+SOX17+(%). FIG. 8D shows representative flow cytometry plots of CD45/CD43 expression in H9 hPSCs (differentiated in accordance with FIG. 8A) on different days (D). FIG. 8E shows a bar graph of quantification of % CD45+CD43+ for H9 hESCs, H1 hESCs, 19-9-11 iPSCs, and 6-9-9 iPSCs. FIG. 8F shows representative flow cytometry plots of CD44/CD43 expression in H9 cell cultures on the indicated days (D). FIG. 8G shows representative flow cytometry plots of CD34/RUNX1c expression in H9 RUNX1c-GFP cell cultures on the indicated days (D).

The resulting day-15 hPSC-derived HSPCs also maintained high viability after freezing and thawing as shown in FIGS. 8I and 8J. FIG. 8H shows representative flow cytometry plots of cell viability before (fresh) and after freezing (frozen) as assessed with calcein-AM stain. FIG. 8I is a bar graph of cell viability (%) for fresh and frozen H9 hPSCs.

Myeloid progenitor and neutrophil differentiation were induced by treating hPSC-derived HSPC cultures with GM-CSF, IL-3 and IL-6 from day 9 as shown in FIG. 9A. FIG. 9A is a schematic diagram of colony forming unit-macrophage (CFU-M) and CFU-granulocyte-macrophage (CFU-GM) analysis at the indicated days during differentiation.

Floating myeloid progenitor cells were collected at different days and presented granulocyte-macrophage and macrophage colony-forming potential, which increased from day 12 to day 18 and decreased thereafter. Neutrophil specification was promoted by treated myeloid progenitors on day 15 with G-CSF. G-CSF treatment significantly decreased the number of CD14+ monocytes/macrophages as compared to GM-CSF as shown in FIGS. 9B-9C. FIG. 9B shows representative flow cytometry plots of CD14/CD45 expression in cells treated with granulocyte-colony stimulating factor (G-CSF) or granulocyte-macrophage CSF (GM-CSF). FIG. 9C is a bar graph of quantification of % CD14+CD45+ expression for day 18 (D18) and day 21 (D21) cultures treated with G-CSF or GM-CSF.

Optimal myeloid progenitors for neutrophil differentiation were identified by collecting floating cells at days 12, 15 and 18 and treating them with G-CSF and AM580, a retinoic acid agonist that promotes neutrophil production from human CD34+ cells as shown in FIG. 9D. FIG. 9D is a schematic diagram of neutrophil differentiation potential evaluation of hPSC-derived myeloid progenitors at the indicated days (D) with G-CSF and AM580 treatment.

The efficiency of neutrophil differentiation increased from day 15 to day 21 and significantly decreased thereafter, perhaps due to the short life-span of neutrophils, as shown in FIGS. 9E-9F. FIG. 9E shows representative flow cytometry plots of CD11b/CD16 expression in myeloid cell cultures treated with G-CSF and AM580 for three, six or nine days and collected on day 12, 15 or 18. FIG. 9F is a bar graph of quantification of % CD11b+CD16+ expression for the myeloid cells collected on day 12, 15 or 18 and analyzed on day (D) 15, 18, 21, 24, or 27.

Day 15 myeloid progenitors treated with G-CSF and AM580 for six days were identified as optimal for neutrophil differentiation. The phenotype of sorted CD16− cells (see FIG. 9G, which shows representative flow cytometry images of CD16 on neutrophils before and after sorting) in the neutrophil differentiation culture was assessed and determined to be mainly composed of ˜20% FcεR1α+ basophils and ˜74% EPX+ eosinophils as shown in FIG. 9H, which shows flow cytometry analysis of CD16− cells sorted for expression of CD10, CD14, FcεR1α, and EPX. Dynamic morphological changes along with the emergence of hematopoietic clusters from day 12 were observed.

The resulting day 21 neutrophils displayed a typical neutrophil morphology (FIGS. 9I-9J) and manifested high expression levels of neutrophil-specific markers, including CD16, CD11b, CD15, CD66b, CD18 and MPO, as compared to their counterparts isolated from human peripheral blood. FIG. 9I shows representative Wright-Giemsa staining of the hPSC-derived and primary peripheral blood neutrophils. Scale bar=5 μm. FIG. 9J shows representative fluorescent staining of DAPI and CD16 of the hPSC-derived and primary peripheral blood neutrophils. Scale bar=10 μm.

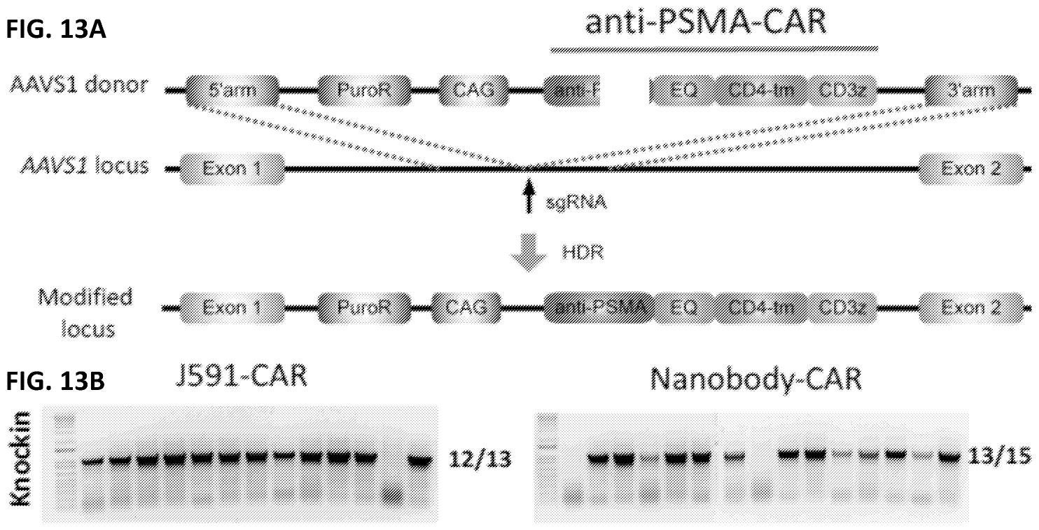

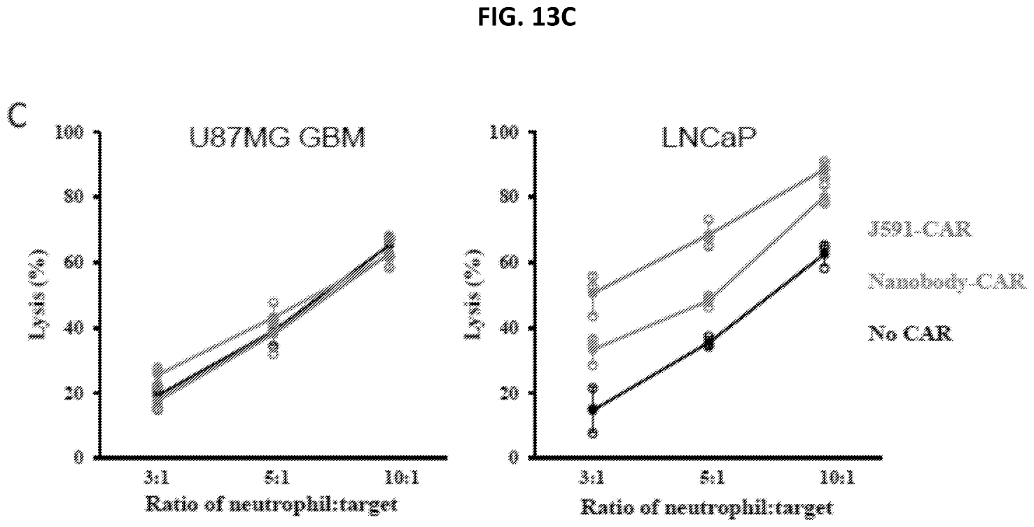

FIGS. 13A-13C: Anti-PSMA CAR-neutrophils derived from hPSCs specifically recognize and kill cancerous lines. (FIG. 13A) Schematic diagrams of PSMA-CAR design and knock-in strategy via Cas9-mediated homology-directed repair (HDR) at the endogenous AAVS1 safe harbor locus. PSMA-CAR is composed of signal peptide, anti-PSMA J591 scFV or nanobody, IgG4-Fc (EQ), CD4 transmembrane (tm) and CD3ζ (CD3z). (FIG. 13B) Genotyping of CAR knockin in hPSCs with a target efficiency of 12 clones from a total of 13 and 13 clones from a total of 15, respectively. (FIG. 13C) CAR-neutrophils were co-cultured with U87MG glioblastoma (GBM) and LNCaP prostate cancer cells at indicated cell ratios for 16 hr and the cytotoxicity of neutrophils was calculated.

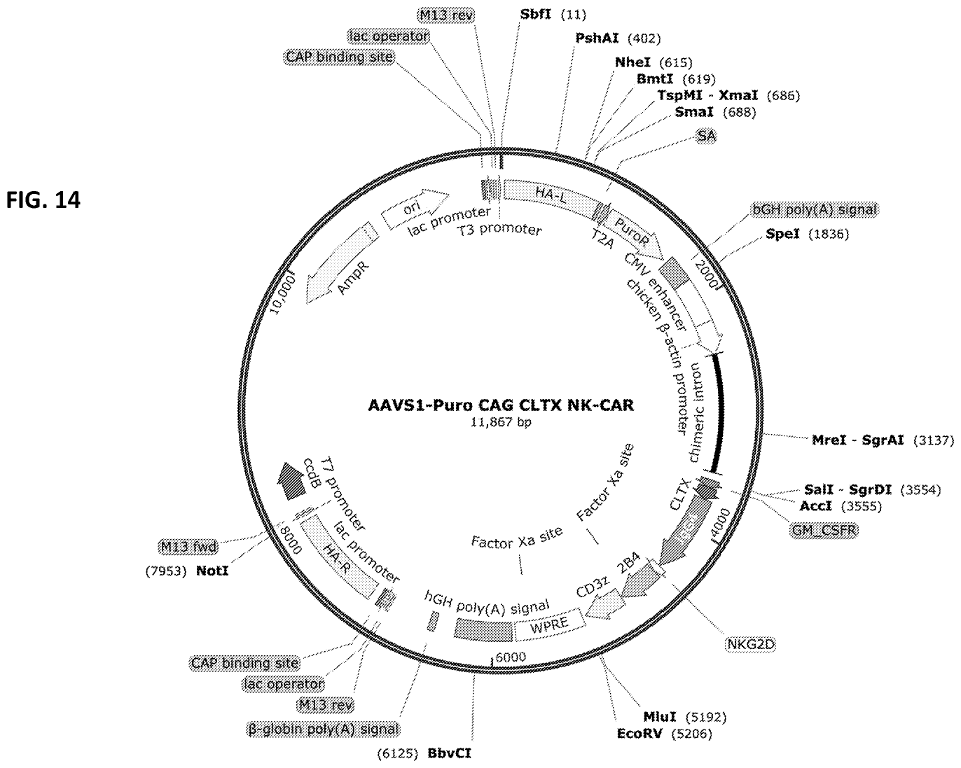

Furthermore, for explanation, FIG. 14 depicts the CLTX NK-CAR plasmid map shown as an illustrative embodiment.

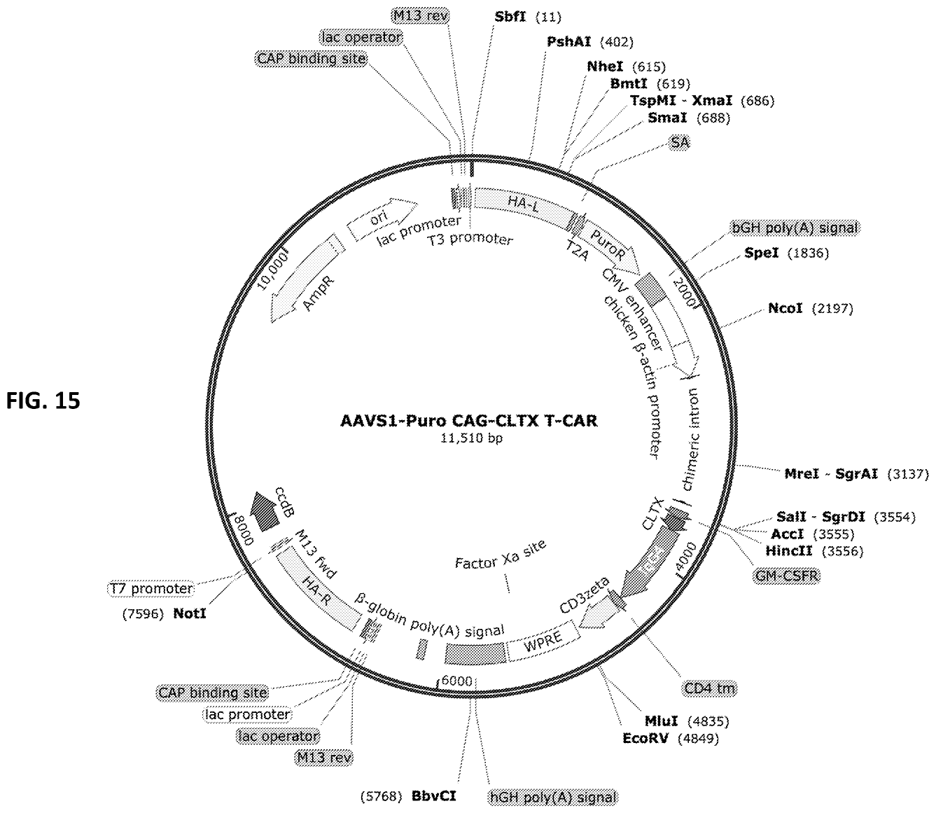

FIG. 15 depicts the CLTX T-CAR plasmid map. The CLTX T-CAR encodes CLTX, the transmembrane domain of CDR, and the intracellular domain of CD3ζ This construct was better than the other two at enhancing anti-tumor cytotoxicity of hPSC-derived neutrophils. The resulting CLTX T-CAR neutrophils presented a typical neutrophil phenotype and killed the targeted tumor cells through specific binding to glioblastoma via MMP2.

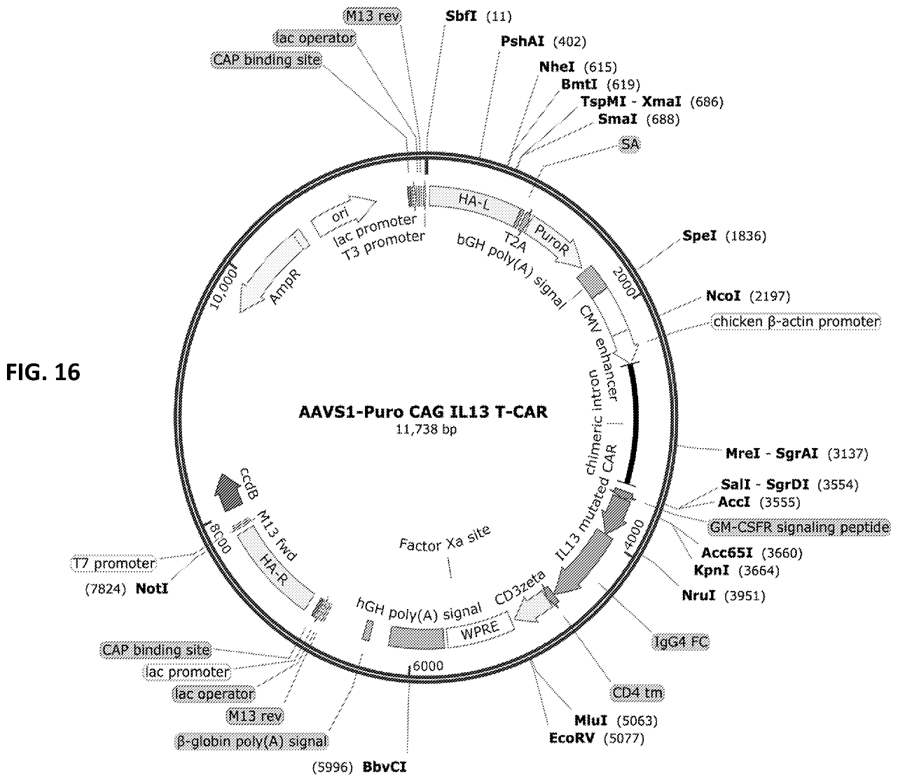

FIG. 16 depicts the IL-13 T-CAR plasmid map shown as an illustrative embodiment for explanation.

While immunotherapy has been developed to cure hematologic malignancies, de novo and acquired resistance to targeted cancer therapy is commonly observed in solid tumors due to the complex and dynamic tumor microenvironment (TME), in which neutrophils are key players (Devlin et al., 2020; Kalafati et al., 2020; Ponzetta et al., 2019). Increased understanding of neutrophil contributions to the TME has increased interest in reprograming and/or depleting pro-tumor neutrophils as an alternative approach to treat cancer (Kalafati et al., 2020). As proof-of-concept, we demonstrate here the feasibility of using synthetic CARs to program and maintain neutrophils as anti-tumor effector cells both in vitro and in vivo, representing a novel neutrophil-targeted immunotherapy that may complement current cancer treatments and boost their efficacy.

Due to the short-life of primary neutrophils and their resistance to genome editing, engineering hPSCs with synthetic CARs would be an ideal approach to produce off-the-shelf CAR-neutrophils. To achieve this goal, here we first implemented an innovative chemically-defined, feeder-free platform for robust production of neutrophils from hPSCs, using stage-specific employment of signaling pathway modulators. We next designed and evaluated three different CAR constructs with NK or T cell-specific transmembrane and intracellular activation domains in enhancing neutrophil-mediated tumor killing activities. CLTX-T-CAR, that contains a GBM-binding peptide chlorotoxin (Qin et al., 2014) and T cell-specific signaling domains, markedly improved antigen-specific tumor cytotoxicity of hPSC-neutrophils both in vitro and in vivo. In future studies, it will be interesting to investigate whether neutrophil-specific transmembrane and activation domains can be harnessed to establish neutrophil-specific CAR constructs. Using an inducible gene knockdown system, we identified membrane protein MMP2 on GBM cells as the target of CLTX-binding and recognition that triggers CAR activation in neutrophils. Molecular mechanism investigation revealed that CLTX-T-CAR triggers known downstream intracellular signaling pathways and gene expression profiles in neutrophils that mediate trogoptosis activity against tumor cells and sustain their anti-tumor N1 phenotype under tumor niche-like conditions.

In summary, the CAR-neutrophil engineering platform described in this study can serve as a scalable strategy to make off-the-shelf neutrophils as potential standardized cellular products for clinical applications in cancer and neutropenia treatment. Given the relative ease of genome editing in hPSCs, other genetic modifications, such as multiple CAR expression and/or inhibitory receptor deletions, can also be performed to achieve optimal therapeutic effects in CAR-neutrophils. Importantly, stable CAR-expressing hPSC lines can be also used to produce off-the-shelf CAR-T and -NK cells that are currently used in clinical trials (Li et al., 2018).

### EXAMPLES

The following examples serve to illustrate the present disclosure. The examples are not intended to limit the scope of the claimed invention in any way.

### Abbreviations

HSPCs: Haematopoietic stem and progenitor cells
AGM: aorta-gonad-mesonephros
NK cells: natural killer cells
HSCs: hematopoietic stem cells
EHT: endothelial-to-hematopoietic transition
HE: hemogenic endothelium
hESCs: human embryonic stem cells
hPSCs: human pluripotent stem cells
BMP4: bone morphogenetic protein 4
VEGF: vascular endothelial growth factor
EPO: erythropoietin
FGF2: fibroblast growth factor 2
CSF3: colony-stimulating factor 3
IL-6: interleukin 6
TPO: thrombopoietin
PVA: polyvinyl alcohol
SCF: stem cell factor
Flt3: FMS-like tyrosine kinase 3/fetal liver kinase 2
HEP: hemogenic endothelium progenitor
VE-cadherin: vascular endothelial cadherin
OP9-DLL4: OP9-Notch ligand delta-like 4
CFU-E: colony forming unit-erythroid
CFU-GM: colony forming unit-granulocyte/macrophage
CFU-M: colony forming unit-macrophage
CFU-GEMM: colony forming unit-multipotential progenitors
GSK3: glycogen synthase kinase 3
DMEM: Dulbecco's modified Eagle medium
TGFβ: transforming growth factor β
G-CSF: granulocyte colony stimulating factor
GM-CSF: granulocyte-macrophage colony stimulating factor
IL-3: interleukin 3
IL-6: interleukin 6
IL-13: interleukin 13
CAR: chimeric antigen receptor
CLTX: chlorotoxin
BSA: bovine serum albumin
PBS: phosphate-buffered saline
FBS: fetal bovine serum
fMLP: formylmethionyl-leucyl-phenylalanine
CFCs: colony-forming cells
GBM: glioblastoma
MMP2: matrix metallopeptidase 2
SOX17: SRY-box transcription factor 17

Statistical analysis. Data are presented as mean±standard error of the mean (s.e.m). Statistical significance was determined by Student's t-test (two-tail) between two groups, and three or more groups were analyzed by one-way analysis of variance (ANOVA). P<0.05 was considered statistically significant.

### Example 1: Cell Culture Medium

The following medium was used to culture and maintain stem cells:
(a) stem cell culture and maintenance medium. mTeSR1 (Stemcell Technologies, 05825), mTeSR Plus (Stemcell Technologies, 85850), E8 (ThermoFisher, A1517001), StemFlex (ThermoFisher, A3349401), etc.
(b) DMEM+Ascorbic acid (used day 0 to day 1). DMEM (ThermoFisher, 11965 or Corning, 10-017-CM) was supplemented with 50-100 μg/ml ascorbic acid (Sigma, A8960)
(c) LaSR basal or similar (used day 0 to day 4, day 0 to day 6, day 0 to day 15, day 1 to day 4, day 1 to day 6, or day 1 to day 15). LaSR basal consists of Advanced DMEM/F12 (ThermoFisher, 12634), 2.5 mM GlutaMAX (ThermoFisher, 35050061), and 50-100 μg/ml ascorbic acid (Sigma, A8960)
(d) Stemline II (Sigma, S0192), StemSpan-XF (Stemcell Technologies, #100-0073) or similar medium (used day 0 to day 20, day 2 to day 20, day 3 to day 20, or day 4 to day 20)

The following small molecules and growth factors were used:
(a) Y27632 (5-10 μM): human pluripotent stem cell culture Cayman Chem, 10005583; Sigma, Y0503; Selleckchem, 51049;

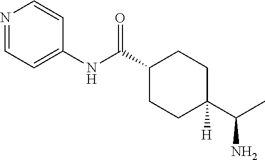 [SMILES: C[C@@H](N)[C@H]1CC[C@H](C(=O)Nc2ccncc2)CC1]

(b) CHIR99021 (1-12 μM): GSK3 inhibitor for meso-endoderm specification Cayman Chem, 13122; Sigma, SML1046; Tocris, 4423;

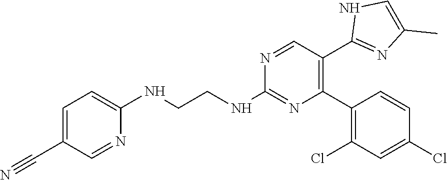 [SMILES: Cc1c[nH]c(-c2cnc(NCCNc3ccc(C#N)cn3)nc2-c2ccc(Cl)cc2Cl)n1]

(c) SB431542 (1-20 μM): TGFβ inhibitor for endothelial-to-hematopoietic transition Cayman Chem, 13031; Selleckchem, 51067; Tocris, 1614;

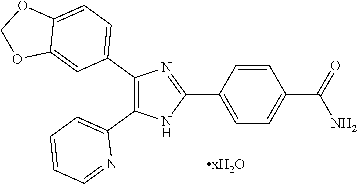 [SMILES: NC(=O)c1ccc(-c2nc(-c3ccc4c(c3)OCO4)c(-c3ccccn3)[nH]2)cc1]

(d) A83-01 (1-20 μM): TGFβ inhibitor for endothelial-to-hematopoietic transition Cayman Chem, 9001799; Sigma, SML0788; Tocris, 2939;

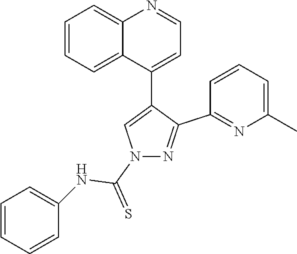 [SMILES: Cc1cccc(-c2nn(C(=S)Nc3ccccc3)cc2-c2ccnc3ccccc23)n1]

(e) Recombinant human VEGF165 (1-100 ng/mL) for endothelial specification Peprotech #100-20; R&D systems, 293-VE; Sigma, H9166;
(f) Recombinant human SCF (1-100 ng/mL) for hematopoietic specification Peprotech #300-07; R&D systems, 255-SC; Sigma, 57901;
(g) Recombinant human Flt3 ligand (1-100 ng/mL) for hematopoietic specification Peprotech #300-19; R&D systems, 308-FK; ThermoFisher, PHC9415;
(h) Recombinant human G-CSF (1-200 ng/ml) for neutrophil specification Peprotech #300-23; R&D systems, 214-CS; Sigma, G0407;
(i) AM580 or AM80 (1-10 μM): Retinoic acid receptor agonist for neutrophil specification
AM580: Cayman Chem, 15261; Selleckchem, 52933; Tocris, 0760;
AM80: Cayman Chem, 71770; Tocris, 3507;

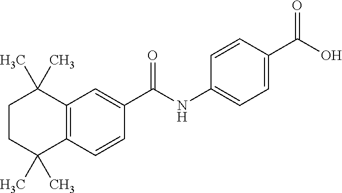 [SMILES: CC1(C)CCC(C)(C)c2cc(C(=O)Nc3ccc(C(=O)O)cc3)ccc21]

(j) Recombinant human granulocyte macrophage-colony stimulating factor (GM-CSF) (1-100 ng/mL) for hematopoietic and neutrophil specification
Peprotech #300-03; BioLegend, 572902; Sigma, G5035;
(k) Recombinant human IL-3 (1-100 ng/mL) for hematopoietic and neutrophil specification
Peprotech #200-03; BioLegend, 578002; Sigma, SRP4134;
(l) Recombinant human IL-6 (1-100 ng/mL) for hematopoietic and neutrophil specification
Peprotech #200-06; BioLegend, 715104; Sigma, GF430-M;

### Example 2: Generation, Maintenance and Differentiation of hPSCs with Different CAR Constructs

CLTX, a peptide containing 36 amino acids and 4 disulfide bonds with a relative molecular mass of 3,996, was originally isolated from Leiurus quinquestriatus scorpion venom (Qin et al., Inhibition of metastatic tumor growth and metastasis via targeting metastatic breast cancer by chlorotoxin-modified liposomes. Mol Pharm (2014). doi:10.1021/mp400691z). CLTX binds selectively to glioblastomas and other tumors (DeBin et al., Purification and characterization of chlorotoxin, a chloride channel ligand from the venom of the scorpion, Am J. Physiol-Cell Physiol 264 (1993)). CLTX is highly toxic to invertebrates but nontoxic to mammals CLTX is a potential tool for tumor-targeting therapy because of its specific binding and other natural properties. CLTX has been reported for tumor-specific delivery of cytotoxic agents, and CLTX has been used to coat a variety of vehicles for delivery of chemotherapeutics and small interfering RNAs for preclinical applications. Recently, Brown et al. reported that CLTX-directed CAR-T cells specifically and effectively targeted glioblastoma. Based on the above investigations, it is believed that CLTX-directed CAR neutrophils can target tumor after migration into inflammatory tumor regions (Wang et al. (2020), supra).

Cas9 was used to engineer hPSCs with three different CAR constructs (IL-13 T-CAR (Kim et al., Bioact Mater 5: 624-635 (2020)), which is an IL-13 receptor α2 (IL13Rα2)-targed quadruple mutant IL-13 (TQM13) T-CAR, CLTX T-CAR (Wang et al. (2020), supra), and CLTX NK-CAR), and differentiate them into CAR-neutrophils for improved immunotherapy. To avoid gene silencing, the CAR constructs were knocked into the adeno-associated virus integration site 1 (AAVS1) safe harbor locus of hPSCs as previously done to express cell-cycle reporters (Chang et al., Fluorescent indicators for continuous and lineage-specific reporting of cell-cycle phases in human pluripotent stem cells. Biotechnol Bioeng bit.27352 (2020). doi:10.1002/bit.27352).

Donor Plasmid Construction

The donor plasmids targeting the AAVS1 locus were constructed as previously described (Chang et al. (2020), supra). Briefly, to generate the CAG-IL13 T-CAR plasmid, the TQM-IL13 CAR fragment (Kim et al. (2020), supra) was amplified from Addgene plasmid #154054 and then cloned into the AAVS1-Puro CAG-FUCCI donor plasmid (Addgene; #136934), replacing the FUCCI. For the CAG-CLTX T-CAR plasmid, the CLTX sequence containing a signal peptide was directly synthesized (GeneWiz) and used to replace the IL-13 sequence in the CAG-IL13 T-CAR. For the CAG-CLTX NK-CAR plasmid, the conjugated NKG2D, 2B4 and CD3-ζ sequences were directly synthesized and used to replace the CD4tm and CD3-ζ sequences in the CAG-CLTX T-CAR. All CAR constructs were sequenced and submitted to Addgene (#157742, #157743 and #157744).

### Example 3: In Vitro Analysis

Nucleofection and Genotyping of hPSCs

To increase cell viability, 10 μM Y27632 was used to treat hPSCs 3-4 hr before nucleofection or overnight. Cells were then singularized by Accutase for 8-10 min, and 1-2.5×10⁶ hPSCs were nucleofected with 3 μg AAVS1 gRNA T2 (Addgene; #41818), 4.5 μg pCas9 GFP (Addgene; #44719), and 6 μg CAR donor plasmids in 100 μl human stem cell nucleofection solution (Lonza; #VAPH-5012) or 200 μl room temperature PBS−/− using program B-016 in a Nucleofector 2b. The nucleofected cells were seeded into one well of a Matrigel-coated, 6-well plate in 3 ml pre-warmed mTeSR plus or mTeSR1 with 10 μM Y27632. Twenty-four hours later, the medium was changed with fresh mTeSR plus or mTeSR1 containing 5 μM Y27632, followed by a daily medium change. When cells were more than 80% confluent, drug selection was performed with 1 puromycin (Puro) for approximately one week, and individual clones were picked using a microscope inside a tissue culture hood and expanded for 2-5 days in each well of a 96-well plate pre-coated with Matrigel, followed by PCR genotyping. The genomic DNA of single clone-derived hPSCs was extracted by scraping cells into 40 μl QuickExtract™ DNA Extraction Solution (Epicentre; #QE09050). 2×GoTaq Green Master Mix (Promega; #7123) was used to perform the genomic DNA PCR. For positive genotyping, the following primer pair with an annealing temperature (Tm) of 65° C. was used: CTGTTTCCCCTTCCCAGGCAGGTCC [SEQ ID NO: 4] and TCGTCGCGGGTGGCGAGGCGCACCG [SEQ ID NO: 5]. For homozygous screening, the following set of primer sequences with a Tm of 60° C. was used: CGGTTAATGTGGCTCTGGTT [SEQ ID NO: 6] and GAGAGAGATGGCTCCAGGAA [SEQ ID NO: 7].

Comparison of bulk RNA sequencing analysis of hPSC-derived neutrophils with the transcriptome of hPSCs and healthy primary neutrophils (Perez et al., Blood 136: 199-209 (2020)) indicated most expression patterns of key surface markers and transcription factors were identical in hPSC-derived neutrophils and primary neutrophils as compared to undifferentiated hPSCs. Consistent with flow cytometry analysis, RNA sequencing confirmed the expression of CD11b, CD15, CD16, CD66b, CD18 and MPO in hPSC-derived and primary neutrophils. hPSC-derived neutrophils demonstrated a relatively low expression level of CEACAM8 and a high expression level of MPO, similar to the expression levels in mature and immature primary neutrophils, respectively. Similarly, other surface receptors, including toll-like receptors (TLRs), adhesion molecules, such as SELL and ITGAX, key transcription factors, such as SPI1, CEBPA, and CEBPE, functional genes, such as PRTN3 and MPO, and genes involved in ROS production, such as NCF2 and NCF4, were expressed at levels similar to those found in primary immature or mature neutrophils.

Despite the similarities, significant differences between hPSC-derived and primary neutrophils were also apparent. For instance, transcription factors associated with early myeloid or granulocyte progenitors, such as RUNX1 and GFI1, retained high expression levels in hPSC-derived neutrophils, indicating an immature phenotype and/or high heterogeneity of neutrophil differentiation cultures. Chemokines and chemo-attractants, including C-X-C motif chemokine receptors (CXCRs) and formyl peptide receptors(FPRs), displayed lower expression levels than intermediate and mature neutrophils, suggesting that hPSC-derived neutrophils may be less sensitive to chemo-attractants than primary neutrophils. Fresh hPSC-neutrophils displayed expression patterns similar to mature, primary neutrophils in terms of N1 and N2 markers, but a unique N1 or N2 transcriptional profile was not observed in all neutrophils since they expressed a subset of N1 and N2 genes at both high and low levels, highlighting the need for more authentic markers enabling the tracking of N1 or N2 human neutrophils. Transcriptional heterogeneity was also observed in immature, intermediate and mature primary neutrophils, indicating the dynamics and plasticity of neutrophils.

Hematopoietic Colony Forming Assay and Wright-Giemsa Staining

Collected cells were grown in 1.5 mL of cytokine-containing MethoCult H4434 medium (StemCell Technologies, Vancouver) at 37° C. After 14 days, the hematopoietic colonies were scored for CFUs according to cellular morphology. To assess the morphology of cells, cells were fixed on glass slides and stained with Wright-Giemsa solution (Sigma-Aldrich).

Flow Cytometry Analysis

Differentiated cells were gently pipetted and filtered through a 70 or 100 μm strainer sitting on a 50 mL tube. The cells were then pelleted by centrifugation and washed three times with PBS−/− solution containing 1% BSA. The cells were stained with appropriately conjugated antibodies for 25 mM at room temperature in the dark and analyzed in an Accuri C6 plus cytometer (Beckton Dickinson) after washing with BSA-containing PBS−/− solution. Flow Jo software was used to process the collected flow data.

Sorted CD16− cells in the neutrophil differentiation culture were mainly composed of ˜20% FcεR1α+ basophils and ˜74% EPX+ eosinophils.

Flow cytometry analysis of anti-IgG4 (SmP)-FITC (FITC=fluorescein isothiocyanate) and IL13Rα2-FITC at different stages of neutrophil differentiation further confirmed stable expression of CARs in hPSCs.

Transwell Migration Assay

In brief, differentiated cells were resuspended in HBSS buffer and allowed to migrate towards fMLP (10 nM and 100 nM) for two hours. Cells that migrated to the lower chamber were released with 0.5 M EDTA and counted using Accuri C6 plus cytometer (Beckton Dickinson). The counts were normalized with the total numbers of cells added to each well. The data were then gated for live cells and analyzed with Flow Jo software.

A transwell-based blood brain barrier (BBB) model involving the use of human cerebral microvascular endothelial cells was used to evaluate further the activity of CAR neutrophils. While CAR-expressing and wild-type hPSC-neutrophils displayed similar transmigration activity across the BBB in response to fMLP, CAR-neutrophils demonstrated higher tumor-killing ability than wild-type neutrophils. Furthermore, CLTX-T-CAR hPSC-neutrophils retained high transmigration ability during their second trafficking across the BBB in response to inflammatory tumor cells, recapitulating many aspects of in vivo inflammation and cancer.

2-D Chemotaxis Assay

Differentiated cells were resuspended in HBBS with 20 mM HEPES and 0.5% FBS and loaded in collagen-coated IBIDI chemotaxis μ-slides and incubated at 37° C. for 30 mM to allow cells to adhere. fMLP (15 μL of 1,000 nM) was loaded into the right reservoir, yielding a final fMLP concentration of 187 nM. Cell migration was recorded every 60 s for 120 min using an LSM 710 (with Ziess EC Plan-NEOFLUAR 10X/0.3 objective) at 37° C. Cells were tracked with the ImageJ plug-in MTrackJ.

Phagocytosis

Phagocytosis was assessed using pHrodo Green E. coli BioParticles Conjugate according to the manufacturer's protocol. In brief, pHrodo Green E. coli beads were resuspended in 2 mL of PBS and sonicated with an ultrasonicator three times. Beads per assay (100 mL) were opsonized by mixing with an opsonizing reagent at a 1:1 ratio and incubated at 37° C. for 1 h. Beads were washed 3 times with mHBSS buffer by centrifugation at 4° C., 1,500 RCF for 15 min then finally resuspended in mHBSS buffer. Differentiated cells were resuspended in 100 μL of opsonized base solution and incubated at 37° C. for 1 h. Then the cells were analyzed with an Accuri C6 plus cytometer (Beckton Dickinson, NJ).

Phagocytosis of tumor cells by neutrophils was significantly reduced after treatment with 5 μM cytochalasin D (CytoD), a chemical that inhibits phagocytosis and neutrophil extracellular trap (NET) formation. PicoGreen staining of NET formation during neutrophil and tumor co-incubation in the absence or presence of the NET inhibitor propofol demonstrated a significant decrease of NET formation in neutrophils treated with 3 μg/ml or more of propofol. While CytoD, N-acetylcysteine (NAC), and propofol significantly blocked tumor lysis by CAR neutrophils, glioblastoma demonstrated higher viability under CytoD and NAC conditions.

Neutrophil-Mediated In Vitro Cytotoxicity Assay

Cell viability was analyzed through flow cytometry. In brief, 100 μL of tumor cells (30,000 cells/mL) mixed with 100 μL of neutrophil cells (90,000 cells/mL, 150,000 cells/mL, 300,000 cells/mL) were placed in 96-well plates. The plates were maintained at 37° C. in a 5% CO₂ incubator for 16 h. The suspension media were transferred into a new round-bottomed, 96-well plate, 50 μL of trypsin-EDTA were added to the wells, and the plates were incubated for 5 min at 37° C., after which the detached cells were transferred to round-bottomed, 96-well plates. The round-bottomed, 96-well plates were centrifuged at 300×g, 4° C. for 4 min, and the supernatants were discarded. Pellets were washed with 200 μL of flow BSA-containing PBS−/− solution and cells were resuspended in 100 μL of BSA-containing PBS−/− solution containing CD45 antibody and Calcein AM for 30 min at room temperature. Then the samples were analyzed by an Accuri C6 plus cytometer (Beckton Dickinson).

### Example 4: Analysis of Binding Specificity of CLTX T-CAR hPSC-Neutrophils

To explore further the molecular mechanisms underlying CLTX-T-CAR-enhanced cytotoxicity against tumor cells, the biological function of potential CLTX ligands, including chloride channels (CLCN3), phospholipid protein annexin A2 (ANXA2), and matrix metallopeptidase 2 (MMP2) was examined. An inducible Cas13d-mediated gene knockdown platform was used to suppress the candidate gene expression. After puromycin selection, approximately 78% of the transfected glioblastoma cells expressed Cas13d as indicated by eGFP expression in the presence of doxycycline (DOX). RT-PCR analysis confirmed the successful knockdown of CLCN3, ANXA2, and MMP2 in U87MG GBM cells. Non-targeting Cas13d single guide RNA (sgRNA) was used as a negative control (e.g., CLCN3 and AXAN2 sgRNAs were used as negative controls for MMP2 sgRNAs), and no obvious cross-gene knockdown effects were observed. Notably, knockdown of MMP2, but not CLCN3 or ANXA2, significantly reduced CLTX-T-CAR neutrophil-mediated tumor cell killing.

To further determine the relationship between CAR-neutrophil activity and MMP2, the gene expression levels of ANXA2, CLCN3 and MMP2 in different tumor cells were assessed. As expected, U87MG, GBM43 and SJ-GBM cells displayed the highest expression levels of MMP2. Linear regression analysis demonstrated tumor lysis of CLTX-T-CAR neutrophils is most likely dependent on MMP2 expression. As compared to GBM cells, MMP2 is expressed at a low to negligible level in normal SVG p12 glial cells and hPSC-derived somatic cells, consistent with their minimal apoptosis in CAR-neutrophil-mediated lysis. These findings further demonstrate that membrane protein MMP2 is required for CLTX-T-CAR recognition and activation of CAR-neutrophils to kill tumor cells. This also suggests the safety of CLTX-T-CAR neutrophils in future clinical application, given the low or negligible MMP2 expression on human normal tissues as compared to GBM.

### Example 5: Analysis of Intracellular Signaling in CLTX T-CAR hPSC-Neutrophils Bound to MMP2-Expressing Tumor Cells

Upon GBM stimulation, CLTX-T-CAR hPSC-neutrophils displayed stronger phosphorylated activation of Syk (p-Syk) than CLTX-NK-CAR hPSC-neutrophils. Notably, a significantly increased ratio of extracellular signal-regulated kinase (Erk) 1/2 (p-Erk1/2), a key signaling mediator involved in lymphoid-mediated cytotoxicity, was also observed. This indicates potential activation of the Syk-vav1-Erk pathway in activated neutrophils.

### Example 6: In Vivo Analysis

Three-Dimensional (3-D) Biomimetic GBM Model

A 3-D biomimetic GBM model was constructed to mimic an in vivo tumor niche-like microenvironment with a dense, extracellular matrix and heterogeneous tumor cell subtypes. As compared to the wild-type control, CLTX-T-CAR hPSC-neutrophils exhibited higher tumor-infiltration and tumor-killing activities under hypoxic (3% O₂) and normoxic (21% O₂) conditions. Pre-treating neutrophils with soluble MMP2 and cytochalasin D (CytoD) significantly reduced tumor-infiltrating activity and tumor cytotoxicity of CAR-neutrophils, consistent with observations in monolayer cell cultures.

To further explore the molecular mechanisms underlying anti- and pro-tumor activities of wild-type and CAR-neutrophils under hypoxia, N1 and N2 phenotype analysis was performed on the isolated neutrophils. Compared with normoxia, hypoxia significantly decreased expression of N1-specific markers, including ICAM-1, iNOS, TNFα and CCL3, and increased N2-specific markers, including CCL2, VEGF, CCL5, and arginase, in wild-type hPSC-neutrophils. On the contrary, CLTX-T-CAR hPSC-neutrophils retained high expression levels of N1 markers under hypoxia.

ELISA was used to detect human cytokine production release in the media after neutrophil-tumor co-culture, including tumor necrosis factor-α (TNFα) and IL-6. Both wild-type and CAR-neutrophils produced TNFα and IL-6 after tumor stimulation, and CAR-neutrophils maintained the highest levels of both cytokines under hypoxia and normoxia. Notably, hypoxia significantly reduced cytokine release in wild-type neutrophils. Taken together, CLTX-T-CAR hPSC-neutrophils sustained an anti-tumor phenotype and retained high transmigration ability and anti-tumor cytotoxicity under tumor-niche mimicking hypoxic conditions. Detailed results are shown in FIGS. 3 and 4.

GBM Xenograft Mouse Model

In a GBM in situ xenograft mouse model, 5×10⁵ luciferase-expressing GBM cells were intracranially injected into the brains of immunodeficient mice. Neutrophils were administrated intratumorally or intravenously to investigate their in vivo tumor-killing activities, in comparison to hPSC-derived CAR-NK cells. Notably, CLTX-T-CAR neutrophils were more effective in killing GBM cells than CLTX-NK-CAR NK cells in vitro, and the combinatory effect between CAR-neutrophils and CAR-NK cells was not observed. Detailed results are shown is FIGS. 5A-5H.

In the intratumoral injection experiment, tumor-bearing mice were administrated a single dose of PBS, 5×10⁶ CLTX-T-CAR hPSC-neutrophils, CLTX-NK-CAR hPSC-NK cells, or corresponding wild-type controls three hours after tumor cell inoculation. Bioluminescent imaging (BLI) was performed to monitor tumor growth weekly after initial imaging on day 3. As compared to PBS-treated mice, treatment with hPSC-derived neutrophils or NK cells significantly reduced tumor burden. hPSC-derived CLTX-NK-CAR NK cells and CLTX-T-CAR neutrophils displayed higher anti-tumor cytotoxicity than their wild-type controls in the mice that maintained a stable body weight. Notably, one of the PBS-treated tumor-bearing mice died at day 30 due to the overgrowth of tumor in the recipient brain. Results are shown in FIGS. 6A-6E.

In the intravenous injection experiment, 5×10⁶ CARneutrophils were administered weekly to the tumor-bearing mice. To track in vivo biodistribution and trafficking of CAR-neutrophils, neutrophils were labeled with Cy5 before systemic injection and fluorescence imaging was performed 1, 5 and 24 hours after systemic administration. Neutrophils trafficked through the whole mouse body in an hour and retained a similar biodistribution 5 hours after neutrophil injection. Compared to wild-type neutrophils, CAR-neutrophils effectively crossed the BBB and trafficked to the GBM xenograft in the mouse brain after 24 hours. No significant changes of body weight were observed across the experimental mouse groups during the intravenous administration study. Consistent with the intratumoral administration study, CAR-neutrophils displayed higher anti-tumor cytotoxicity than PBS and wild-type controls in mice according to BLI analysis as well as brightfield and H&E staining images of GBM xenografts. Notably, tumor-bearing mice treated with CLTX-T-CAR hPSC-neutrophils demonstrated a significantly reduced tumor burden as compared to those treated with CAR-NK cells, suggesting the superior ability of neutrophils in crossing the BBB and penetrating GBM xenograft in mice.

In contrast to CAR-neutrophils, weekly administration of wild-type hPSC-neutrophils or peripheral blood (PB) neutrophils significantly promoted the growth of tumor in the brain with or without CAR-NK cells and resulted in the death of tumor-bearing mice as early as day 21. Despite the rarity of extra-neural metastasis of GBM and unknown pathogenesis, systemic metastasis occurred in the tumor-bearing mice treated with wild-type hPSC-derived or PB-neutrophils as determined by BLI images of various ex vivo organs and/or tissues, suggesting a potential role of neutrophils in the extracranial metastasis of human GBM. Experimental results are detailed in FIGS. 7A-7H and FIGS. 12A-12G.

Human cytokine release, including TNFα and IL-6, was measured in the plasma of different experimental mouse groups. All non-PBS experimental groups produced detectable TNFα and IL-6 in plasma from day 5 to day 26, and CAR-neutrophils maintained the highest levels of both cytokines.

Human neutrophils were harvested from mouse blood and subjected for anti-tumor (N1) or pro-tumor (N2) phenotype analysis in an effort to explore further the underlying mechanism of neutrophil-mediated metastasis. Tumor xenografts significantly decreased expression of N1-specific markers, including iNOS and TNFα, and increased N2-specific markers, including VEGF and arginase in wild-type hPSC- or PB-neutrophils. On the contrary, CLTX-T-CAR hPSC-neutrophils retained high expression levels of N1 markers, which is consistent with their strong anti-tumor cytotoxicity and cytokine release in tumor-bearing mice. Collectively, the data demonstrate that hPSC-derived CAR-neutrophils can sustain anti-tumor activity, efficiently kill tumor cells under various tumor niche-like conditions, and prolong animal survival compared to peripheral blood neutrophils, hPSC neutrophils, and CLTX-NK CAR cells.

Experimental Model and Subject Details

Donor plasmid construction. The donor plasmids targeting AAVS1 locus were constructed as previously described (Chang et al., 2020). Briefly, to generate the CAG-IL13 T-CAR plasmid, the TQM-IL13 CAR fragment (Kim et al., 2020) was amplified from Addgene plasmid #154054 and then cloned into the AAVS1-Puro CAG-FUCCI donor plasmid (Addgene; #136934), replacing the FUCCI. For CAG-CLTX T-CAR plasmid, the chlorotoxin sequence containing a signal peptide was directly synthesized (GeneWiz) and used to replace the IL-13 sequence in CAG-IL13 T-CAR. For CAG-CLTX NK-CAR plasmid, the conjugated NKG2D, 2B4 and CD3-ζ sequence was directly synthesized and used to replace the CD4tm and CD3-ζ sequence in CAG-CLTX T-CAR. All CAR constructs were sequenced and submitted to Addgene (#157742, #157743 and #157744).

Maintenance and differentiation of hPSCs. H9, H1, 6-9-9 and 19-9-11 hPSC lines were obtained from WiCell and maintained on Matrigel- or iMatrix 511-coated plates in mTeSR plus medium. For neutrophil differentiation, hPSCs were dissociated with 0.5 mM EDTA and seeded onto iMatrix 511-coated 24-well plate at a cell density between 10,000 and 80,000 cells/cm² in mTeSR plus medium with 5 μM Y27632 for 24 hours (day −1). At day 0, cells were treated with 6 μM CHIR99021 (CHIR) in DMEM medium supplemented with 100 μg/mL ascorbic acid (DMEM/Vc), followed by a medium change with LasR basal medium from day 1 to day 4.50 ng/mL VEGF was added to the medium from day 2 to day 4. At day 4, medium was replaced by Stemline II medium (Sigma) supplemented with 10 μM SB431542, 25 ng/mL SCF and FLT3L. On day 6, SB431542-containing medium was aspirated and cells were maintained in Stemline II medium with 50 ng/mL SCF and FLT3L. At day 9 and day 12, the top half medium was aspirated and changed with 0.5 ml fresh Stemline II medium containing 50 ng/mL SCF, 50 ng/mL FLT3L and 25 ng/mL GM-CSF. Day 15 floating cells were gently harvested and filtered for terminal neutrophil differentiation in Stemline II medium supplemented with 1× GlutaMAX, 150 ng/mL G-CSF, and 2.5 μM retinoic acid agonist AM580. Half medium change was performed every 3 days, and mature neutrophils could be harvested for analysis starting from day 21.

Nucleofection and genotyping of hPSCs. To increase cell viability, 10 μM Y27632 was used to treat hPSCs 3-4 hr before nucleofection or overnight. Cells were then singularized by Accutase for 8-10 min, and 1-2.5×10⁶ hPSCs were nucleofected with 6 μg SpCas9 AAVS1 gRNA T2 (Addgene; #79888) and 6 μg CAR donor plasmids in 100 μl human stem cell nucleofection solution (Lonza; #VAPH-5012) or 200 μl room temperature PBS−/− using program B-016 in a Nucleofector 2b. The nucleofected cells were seeded into one well of a Matrigel-coated 6-well plate in 3 ml pre-warmed mTeSR plus or mTeSR1 with 10 μM Y27632. 24 hr later, the medium was changed with fresh mTeSR plus or mTeSR1 containing 5 μM Y27632, followed by a daily medium change. When cells were more than 80% confluent, drug selection was performed with 1 μg/ml puromycin (Puro) for 24 hr. Once cells recovered, 1 μg/ml Puro was applied continuously for about 1 week. Individual clones were then picked using a microscope inside a tissue culture hood and expanded for 2-5 days in each well of a 96-well plate pre-coated with Matrigel, followed by a PCR genotyping. The genomic DNA of single clone-derived hPSCs was extracted by scraping cells into 40 μl QuickExtract™ DNA Extraction Solution (Epicentre; #QE09050). 2×GoTaq Green Master Mix (Promega; #7123) was used to perform the genomic DNA PCR.

For positive genotyping, the following primer pair with an annealing temperature Tm of 65° C. was used: CTGTTTCCCCTTCCCAGGCAGGTCC [SEQ ID NO: 4] and TCGTCGCGGGTGGCGAGGCGCACCG [SEQ ID NO: 5]. For homozygous screening, we used the following set of primer sequences: CGGTTAATGTGGCTCTGGTT [SEQ ID NO: 6] and GAGAGAGATG GCTCCAGGAA [SEQ ID NO: 7] with an annealing temperature Tm of 60° C.

Hematopoietic Colony Forming Assay and Wright-Giemsa Staining. Collected cells were grown in 1.5 mL of cytokine containing MethoCult H4434 medium (StemCell Technologies, Vancouver) at 37° C. The hematopoietic colonies were scored for colony forming units (CFUs) according to cellular morphology. To assess the morphology of cells, neutrophils were fixed on glass slides and stained with Wright-Giemsa solution (Sigma-Aldrich).

Flow cytometry analysis. Differentiated cells were gently pipetted and filtered through a 70 or 100 μm strainer sitting on a 50 mL tube. The cells were then pelleted by centrifugation and washed twice with PBS−/− solution containing 1% bovine serum albumin (BSA). The cells were stained with appropriate conjugated antibodies for 25 mM at room temperature in dark, and analyzed in an Accuri C6 plus cytometer (Beckton Dickinson) after washing with BSA-containing PBS−/− solution. FlowJo software was used to process the collected flow data.

Transwell migration assay. Differentiated neutrophils were resuspended in HBSS buffer and allowed to migration for 2 hr towards fMLP (10 nM and 100 nM). Cells that migrated to the lower chamber were released with 0.5 M EDTA and counted using Accuri C6 plus cytometer (Beckton Dickinson). Live neutrophils were gated and analyzed in FlowJo software. The neutrophil counts were then normalized by the total numbers of cells added to each well.

2D chemotaxis assay. Differentiated neutrophils were resuspended in HBBS with 20 mM HEPES and 0.5% FBS, and loaded into collagen-coated IBIDI chemotaxis μ-slides, which were then incubated at 37° C. for 30 mM for cells to adhere. 15 μL of 1000 nM fMLP was loaded into the right reservoir yielding a final fMLP concentration of 187 nM. Cell migration was recorded every 60 s for a total of 120 min using LSM 710 (with Ziess EC Plan-NEOFLUAR 10×/0.3 objective) at 37° C. Cells were tracked with ImageJ plug-in MTrackJ.

Phagocytosis. Phagocytosis was assessed using pHrodo Green E. coli BioParticles Conjugate according to the manufacturer's protocol. In brief, pHrodo Green E. coli beads were resuspended in 2 mL of PBS and sonicated with an ultrasonicator 3 times. Beads per assay (100 mL) were opsonized by mixing with opsonizing reagent at a 1:1 ratio and incubated at 37° C. for 1 hr. Beads were washed 3 times with mHBSS buffer by centrifugation at 4° C., 1,500 RCF for 15 mM, and resuspended in mHBSS buffer. Differentiated neutrophils were resuspended in 100 μL of opsonized based solution and incubated at 37° C. for 1 hr, followed by flow cytometry analysis using a Accuri C6 plus cytometer (Beckton Dickinson).

Neutrophil-mediated in vitro cytotoxicity assay. The cell viability was analyzed by flow cytometry. In brief, 100 μL of tumor cells (50000 cells/mL) were mixed with 100 μL of 150,000, 250,000 and 500,000 cells/mL neutrophils in 96 well plates, and then incubated at 37° C., 5% CO₂ for 24 hr. To harvest all the cells, cell-containing media was firstly transferred into a new round-bottom 96-well plate, and 50 μL of trypsin-EDTA was added to the empty wells. After a 5-min incubation at 37° C., attached cells were dissociated and transferred into the same wells of round-bottom 96-well plate with suspension cultures. All of the cells were pelleted by centrifuging the 96-well plate at 300×g, 4° C. for 4 min, and washed with 200 μL of PBS−/− solution containing 0.5% BSA. The pelleted cell mixtures were then stained with CD45 antibody and Calcein AM for 30 min at room temperature, and analyzed in the Accuri C6 plus cytometer (Beckton Dickinson).

Inducible gene knockdown in glioblastoma cells. To achieve inducible gene knockdown in glioblastoma cells, a PiggyBac (PB)-based all-in-one inducible Cas13d plasmid (Addgene #155184) was implemented. The CLCN3, ANXA2, and MMP2 targeting sgRNAs were designed using an online tool (https://cas13design.nygenome.org/) and cloned into the Cas13d backbone to make all-in-one CLCN3, ANXA2, and MMP2 targeting plasmids. The resulting Cas13d plasmids along with a hyPBase plasmid (kindly provided by Dr. Pentao Liu) were then introduced into U87MG cells via PEI transfection. After 2 to 4 days, transfected cells were treated with 5 μg/mL puromycin for one or two days to select drug-resistant tumor cells. After recovering, survived tumor cells were maintained under puromycin condition to avoid potential silencing of the integrated transgenes.

Conjugate formation assay. To visualize immunological synapses, 100 μL of U87MG cells (50,000 cells/mL) were seeded onto wells of 96-well plate and incubated at 37° C. for 12 hours, allowing them to attach. 100 μL neutrophils (500,000 cells/mL) were then added onto the target U87MG cells and incubated for 6 hours before fixation with 4% paraformaldehyde (in PBS). Cytoskeleton staining was then performed using an F-actin Visualization Biochem Kit (Cytoskeleton Inc.).

Trogocytosis assay. The transfer of membrane and cellular content from tumor cell to neutrophils was investigated using both microscope and flow cytometry analysis. Target tumor cells were labeled with Calcein-AM (1 μM) for 30 min at 37° C. After washing with PBS, Calcein-AM labelled tumor cells were then incubated with neutrophils at a neutrophil-to-tumor ratio of 10:1. At different time points (between 0 to 6 hours), the resulting co-culture samples were imaged by a Leica DMi-8 fluorescent microscope. The floating neutrophils were collected for CD45 staining and analyzed in the Accuri C6 plus flow cytometer (Beckton Dickinson) after washing with PBS−/− solution containing 0.5% BSA.

Measurement of reactive oxygen species (ROS) production in neutrophils. 100 μL of U87MG cells (30,000 cells/mL) were seeded into wells of a 96-well plate 12 hours before adding neutrophils at a neutrophil-to-tumor ratio of 10:1. After co-incubation for 12 hours, the resulting cell mixture was treated with 10 μM H₂DCFDA at 37° C. for 50 min and then the fluorescence emission signal (480-600 nm) was collected in a SpectraMax iD3 microplate reader (Molecular Devices, Sunnyvale, CA, USA) with an excitation wavelength of 475 nm.

Blood-brain-barrier (BBB) transmigration assay. In vitro BBB model was constructed with HBEC-5i cells in a transwell cell culture plate. Briefly, HBEC-5i cells (1×10⁵ cells/well) were seeded onto the upper chamber of the transwell pre-coated with gelatin (1% w:v) in 24-well transwell plates (8 μm pore size, 6.5 mm diameter, Corning), and maintained in DMEM/F12 medium containing 10% FBS. 2×10⁵ neutrophils were then added to the upper chamber, and FBS-free medium with or without fMLP (10 nM) was added to the lower chamber. After 3 hours of incubation, cell cultures were collected from the upper or lower chamber for the calculation of neutrophil numbers. For the cytotoxicity analysis, 2×10⁴ U87MG cells were seeded at the lower chamber 12 hours before adding neutrophils (2×10⁵ cells) to the upper chamber, and FBS-free medium with fMLP (10 nM) was then added to the lower chamber. After 12 hours of incubation, tumor cell viability was determined by flow cytometry analysis. For the second migration analysis, 2×10⁵ neutrophils from the bottom chamber of first migration were seeded on the upper chamber of second transwell BBB model, and the migrated neutrophils toward target tumor cells in the bottom chamber was quantified.

Neutrophil infiltration of 3D tumor spheroids. The 3D tumor spheroids were obtained using the hanging drop method. Briefly, U87MG cells were suspended in MEM medium with 10% FBS and 0.3% methylcellulose at 2×10⁶ cells/mL and deposited onto an inverted lid of 96-well plate as an individual drop using a 20 μL pipettor. The cover lid was then placed back onto the PBS-filled bottom chamber and incubated at 37° C. and 5% CO₂. The hanging drops were monitored daily until cell aggregates were formed in ˜5-7 days. Each cell aggregate was transferred to a single well of a 24-well plate for the substream analysis. To assess the tumor penetration capability of CLTX T-CAR neutrophils, 2×10⁵ neutrophils/well were added to the wells of 24-well plate and incubated with the tumor spheroids. After co-incubation for 24 hours, the tumor spheroids were fixed and stained for CD45 and DAPI. For the cytotoxicity analysis, the mixture of neutrophils and tumor spheroids were stained with 1 μM Calcein-AM and 1 μM propidium iodide (PI). The stained cells were then imaged using a Leica DMi-8 fluorescent microscope.

GBM xenograft studies. All the mouse experiments were approved by the Purdue Animal Care and Use Committee (PACUC). The immunodeficient NOD.Cg-RAG^{1tm1Mom}IL2rg^{tm1Wjl}/SzJ (NRG) mice were bred and maintained by the Biological Evaluation Core at the Purdue University Center for Cancer Research. In situ xenograft murine models were constructed via intracranial injection of 5×10⁵ luciferase-expressing GBM cells into the brain of immunodeficient mice. 5×10⁶ neutrophils were administered via either intratumoral or intravenous injection to evaluate their in vivo anti-tumor activities. Tumor burden was monitored by bioluminescence imaging (BLI) and weight body of experimental mice was measured about once per week.

Statistical analysis. Data are presented as mean±standard deviation (SD). Statistical significance was determined by Student's t-test (two-tail) between two groups, and three or more groups were analyzed by one-way analysis of variance (ANOVA). P<0.05 was considered statistically significant.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCE

1. Afonso, P. V., McCann, C. P., Kapnick, S. M., and Parent, C. A. (2013). Discoidin domain receptor 2 regulates neutrophil chemotaxis in 3D collagen matrices. Blood 121, 1644-1650.
2. Bao, X., Lian, X., Dunn, K. K., Shi, M., Han, T., Qian, T., and Palecek, S. P. (2015). Chemically-defined albumin-free differentiation of human pluripotent stem cells to endothelial progenitor cells. Stem Cell Res. 15, 122-129.
3. Bertrand, J. Y., Chi, N. C., Santoso, B., Teng, S., Stainier, D. Y. R., and Traver, D. (2010). Haematopoietic stem cells derive directly from aortic endothelium during development. Nature 464, 108-111.
4. Boisset, J.-C., van Cappellen, W., Andrieu-Soler, C., Galjart, N., Dzierzak, E., and Robin, C. (2010). In vivo imaging of haematopoietic cells emerging from the mouse aortic endothelium. Nature 464, 116-120.
5. Brok-Volchanskaya, V. S., Bennin, D. A., Suknuntha, K., Klemm, L. C., Huttenlocher, A., and Slukvin, I. (2019a). Effective and Rapid Generation of Functional Neutrophils from Induced Pluripotent Stem Cells Using ETV2-Modified mRNA. Stem Cell Reports 13, 1099-1110.
6. Brok-Volchanskaya, V. S., Bennin, D. A., Suknuntha, K., Klemm, L. C., Huttenlocher, A., and Slukvin, I. (2019b). Effective and Rapid Generation of Functional Neutrophils from Induced Pluripotent Stem Cells Using ETV2-Modified mRNA. Stem Cell Reports 13, 1099-1110.
7. Cao, X., Yakala, G. K., van den Hil, F. E., Cochrane, A., Mummery, C. L., and Orlova, V. V. (2019). Differentiation and Functional Comparison of Monocytes and Macrophages from hiPSCs with Peripheral Blood Derivatives. Stem Cell Reports 12, 1282-1297.
8. Chan, X. Y., Volkova, E., Eoh, J., Black, R., Fang, L., Gorashi, R., Song, J., Wang, J., Elliott, M. B., Barreto-Ortiz, S. F., et al. (2021). HIF2A gain-of-function mutation modulates the stiffness of smooth muscle cells and compromises vascular mechanics. IScience 24.
9. Clarke, R. L., Yzaguirre, A. D., Yashiro-Ohtani, Y., Bondue, A., Blanpain, C., Pear, W. S., Speck, N. A., and Keller, G. (2013). The expression of Sox17 identifies and regulates haemogenic endothelium. Nat. Cell Biol. 15, 502-510.
10. Coffelt, S. B., Kersten, K., Doornebal, C. W., Weiden, J., Vrijland, K., Hau, C. S., Verstegen, N. J. M., Ciampricotti, M., Hawinkels, L. J. A. C., Jonkers, J., et al. (2015). IL-17-producing γδ T cells and neutrophils conspire to promote breast cancer metastasis. Nature 522, 345-348.
11. Coffelt, S. B., Wellenstein, M. D., and De Visser, K. E. (2016). Neutrophils in cancer: Neutral no more. Nat. Rev. Cancer 16, 431-446.
12. Devlin, J. C., Zwack, E. E., Tang, M. S., Li, Z., Fenyo, D., Torres, V. J., Ruggles, K. V., and Loke, P. (2020). Distinct Features of Human Myeloid Cell Cytokine Response Profiles Identify Neutrophil Activation by Cytokines as a Prognostic Feature during Tuberculosis and Cancer. J. Immunol. 204, 3389-3399.
13. Eruslanov, E. B., Singhal, S., and Albelda, S. M. (2017). Mouse versus Human Neutrophils in Cancer: A Major Knowledge Gap. Trends in Cancer 3, 149-160.
14. Feins, S., Kong, W., Williams, E. F., Milone, M. C., and Fraietta, J. A. (2019). An introduction to chimeric antigen receptor (CAR) T-cell immunotherapy for human cancer. Am. J. Hematol. 94, S3-S9.
15. Fidanza, A., Romano, N., Ramachandran, P., Tamagno, S., Lopez-Yrigoyen, M., Taylor, A. H., Easterbrook, J., Henderson, B., Axton, R., Henderson, N. C., et al. (2019). Single cell transcriptome analysis reveals markers of naïve and lineage-primed hematopoietic progenitors derived from human pluripotent stem cells. BioRxiv 602565.
16. Fridlender, Z. G., Sun, J., Kim, S, Kapoor, V., Cheng, G., Ling, L., Worthen, G. S., and Albelda, S. M. (2009). Polarization of Tumor-Associated Neutrophil Phenotype by TGF-β: “N1” versus “N2” TAN. Cancer Cell 16, 183-194.
17. Huang, X., Trinh, T., and Broxmeyer, H. E. (2018). Hypoxia Signaling Pathway in Stem Cell Regulation: Good and Evil. Curr. Stem Cell Reports 4, 149-157.
18. Huo, X., Li, H., Li, Z., Yan, C., Agrawal, I., Mathavan, S., Liu, J., and Gong, Z. (2019). Transcriptomic profiles of tumor-associated neutrophils reveal prominent roles in enhancing angiogenesis in liver tumorigenesis in zebrafish. Sci. Rep. 9.
19. Ilie, M., Hofman, V., Ortholan, C., Bonnetaud, C., Coëlle, C., Mouroux, J., and Hofman, P. (2012). Predictive clinical outcome of the intratumoral CD66b-positive neutrophil-to-CD8-positive T-cell ratio in patients with resectable nonsmall cell lung cancer. Cancer 118, 1726-1737.
20. Itatani, Y., Yamamoto, T., Zhong, C., Molinolo, A. A., Ruppel, J., Hegde, P., Taketo, M. M., and Ferrara, N. (2020). Suppressing neutrophil-dependent angiogenesis abrogates resistance to anti-VEGF antibody in a genetic model of colorectal cancer. Proc. Natl. Acad. Sci. U.S.A. 117, 21598-21608.
21. Itoh, Y. (2015). Membrane-type matrix metalloproteinases: Their functions and regulations. Matrix Biol.
22. Jaillon, S., Ponzetta, A., Di Mitri, D., Santoni, A., Bonecchi, R., and Mantovani, A. (2020). Neutrophil diversity and plasticity in tumour progression and therapy. Nat. Rev. Cancer 20, 485-503.
23. June, C. H., and Sadelain, M. (2018). Chimeric Antigen Receptor Therapy. N. Engl. J. Med. 379, 64-73.
24. June, C. H., O'Connor, R. S., Kawalekar, O. U., and Milone, M. C. (2018). CAR T cell immunotherapy for human cancer. Science (80-.). 359, 1361-1365.
25. Kalafati, L., Kourtzelis, I., Schulte-Schrepping, J., Li, X., Hatzioannou, A., Grinenko, T., Hagag, E., Sinha, A., Has, C., Dietz, S., et al. (2020). Innate Immune Training of Granulopoiesis Promotes Anti-tumor Activity. Cell 183, 771-785.e12.
26. Kargl, J., Zhu, X., Zhang, H., Yang, G. H. Y., Friesen, T. J., Shipley, M., Maeda, D. Y., Zebala, J. A., McKay-Fleisch, J., Meredith, G., et al. (2019). Neutrophil content predicts lymphocyte depletion and anti-PD1 treatment failure in NSCLC. JCI Insight 4.
27. Kim, I., Saunders, T. L., and Morrison, S. J. (2007). Sox17 Dependence Distinguishes the Transcriptional Regulation of Fetal from Adult Hematopoietic Stem Cells. Cell 130, 470-483.
28. Kissa, K., and Herbomel, P. (2010). Blood stem cells emerge from aortic endothelium by a novel type of cell transition. Nature 464, 112-115.
29. Klichinsky, M., Ruella, M., Shestova, O., Lu, X. M., Best, A., Zeeman, M., Schmierer, M., Gabrusiewicz, K., Anderson, N. R., Petty, N. E., et al. (2020). Human chimeric antigen receptor macrophages for cancer immunotherapy. Nat. Biotechnol. 1-7.
30. Lachmann, N., Ackermann, M., Frenzel, E., Liebhaber, S., Brennig, S., Happle, C., Hoffmann, D., Klimenkova, O., Lüttge, D., Buchegger, T., et al. (2015). Large-scale hematopoietic differentiation of human induced pluripotent stem cells provides granulocytes or macrophages for cell replacement therapies. Stem Cell Reports 4, 282-296.
31. Lecot, P., Sarabi, M., Pereira Abrantes, M., Mussard, J., Koenderman, L., Caux, C., Bendriss-Vermare, N., and Michallet, M. C. (2019). Neutrophil Heterogeneity in Cancer: From Biology to Therapies. Front. Immunol. 10.
32. Li, L., Qi, X., Sun, W., Abdel-Azim, H., Lou, S., Zhu, H., Prasadarao, N. V, Zhou, A., Shimada, H., Shudo, K., et al. (2016). Am80-GCSF synergizes myeloid expansion and differentiation to generate functional neutrophils that reduce neutropenia-associated infection and mortality. EMBO Mol. Med. 8, 1340-1359.
33. Li, Y., Hermanson, D. L., Moriarity, B. S., and Kaufman, D. S. (2018). Human iPSC-Derived Natural Killer Cells Engineered with Chimeric Antigen Receptors Enhance Anti-tumor Activity. Cell Stem Cell 23, 181-192.e5.
34. Lian, X., Bao, X., Al-Ahmad, A., Liu, J., Wu, Y., Dong, W., Dunn, K. K., Shusta, E. V., and Palecek, S. P. (2014). Efficient Differentiation of Human Pluripotent Stem Cells to Endothelial Progenitors via Small-Molecule Activation of WNT Signaling. Stem Cell Reports 3, 804-816.
35. Liang, J., Piao, Y., Holmes, L., Fuller, G. N., Henry, V., Tiao, N., and De Groot, J. F. (2014). Neutrophils promote the malignant glioma phenotype through S100A4. Clin. Cancer Res. 20, 187-198.
36. Lim, W. A., and June, C. H. (2017). The Principles of Engineering Immune Cells to Treat Cancer. Cell 168, 724-740.
37. Matlung, H. L., Babes, L., Zhao, X. W., van Houdt, M., Treffers, L. W., van Rees, D. J., Franke, K., Schornagel, K., Verkuijlen, P., Janssen, H., et al. (2018). Neutrophils Kill Antibody-Opsonized Cancer Cells by Trogoptosis. Cell Rep. 23, 3946-3959.e6.
38. McDermott, D. H., De Ravin, S. S., Jun, H. S., Liu, Q., Long Priel, D. A., Noel, P., Takemoto, C. M., Diode, T., Paul, S. M., Dunsmore, K. P., et al. (2010). Severe congenital neutropenia resulting from G6PC3 deficiency with increased neutrophil CXCR4 expression and myelokathexis. Blood 116, 2793-2802.
39. Mehta, R. S., and Rezvani, K. (2018). Chimeric antigen receptor expressing natural killer cells for the immunotherapy of cancer. Front. Immunol. 9.
40. Ng, E. S., Azzola, L., Bruveris, F. F., Calvanese, V., Phipson, B., Vlahos, K., Hirst, C., Jokubaitis, V. J., Yu, Q. C., Maksimovic, J., et al. (2016). Differentiation of human embryonic stem cells to HOXA+ hemogenic vasculature that resembles the aorta-gonad-mesonephros. Nat. Biotechnol. 34, 1168-1179.
41. Oatley, M., Boltikbasi, Ö. V., Svensson, V., Shvartsman, M., Ganter, K., Zirngibl, K., Pavlovich, P. V., Milchevskaya, V., Foteva, V., Natarajan, K. N., et al. (2020). Single-cell transcriptomics identifies CD44 as a marker and regulator of endothelial to haematopoietic transition. Nat. Commun. 11.
42. Ponzetta, A., Carriero, R., Carnevale, S., Barbagallo, M., Molgora, M., Perucchini, C., Magrini, E., Gianni, F., Kunderfranco, P., Polentarutti, N., et al. (2019). Neutrophils Driving Unconventional T Cells Mediate Resistance against Murine Sarcomas and Selected Human Tumors. Cell 178, 346-360.e24.
43. Qin, C., He, B., Dai, W., Zhang, H., Wang, X., Wang, J., Zhang, X., Wang, G., Yin, L., and Zhang, Q. (2014). Inhibition of metastatic tumor growth and metastasis via targeting metastatic breast cancer by chlorotoxin-modified liposomes. Mol. Pharm.
44. Rankin, E. B., Nam, J. M., and Giaccia, A. J. (2016). Hypoxia: Signaling the Metastatic Cascade. Trends in Cancer 2, 295-304.
45. Rosen, J., Blau, T., Grau, S. J., Barbe, M. T., Fink, G. R., and Galldiks, N. (2018). Extracranial Metastases of a Cerebral Glioblastoma: A Case Report and Review of the Literature. Case Rep. Oncol. 11, 591-600.
46. Saeki, K., Saeki, K., Nakahara, M., Matsuyama, S., Nakamura, N., Yogiashi, Y., Yoneda, A., Koyanagi, M., Kondo, Y., and Yuo, A. (2009). A Feeder-Free and Efficient Production of Functional Neutrophils from Human Embryonic Stem Cells. Stem Cells 27, 59-67.
47. Sagiv, J. Y., Michaeli, J., Assi, S., Mishalian, I., Kisos, H., Levy, L., Damti, P., Lumbroso, D., Polyansky, L., Sionov, R. V., et al. (2015). Phenotypic diversity and plasticity in circulating neutrophil subpopulations in cancer. Cell Rep. 10, 562-573.
48. Seo, Y. J., Cho, W. H., Kang, D. W., and Cha, S. H. (2012). Extraneural metastasis of glioblastoma multiforme presenting as an unusual neck mass. J. Korean Neurosurg. Soc. 51, 147-150.
49. Shaul, M. E., Levy, L., Sun, J., Mishalian, I., Singhal, S., Kapoor, V., Horng, W., Fridlender, G., Albelda, S. M., and Fridlender, Z. G. (2016). Tumor-associated neutrophils display a distinct N1 profile following TGFβ modulation: A transcriptomics analysis of pro- vs. antitumor TANs. Oncoimmunology 5.
50. Smith, J. R., Maguire, S., Davis, L. A., Alexander, M., Yang, F., Chandran, S., ffrench-Constant, C., and Pedersen, R. A. (2008). Robust, Persistent Transgene Expression in Human Embryonic Stem Cells Is Achieved with AAVS1-Targeted Integration. Stem Cells 26, 496-504.
51. Sweeney, C. L., Teng, R., Wang, H., Merling, R. K., Lee, J., Choi, U., Koontz, S., Wright, D. G., and Malech, H. L. (2016). Molecular Analysis of Neutrophil Differentiation from Human Induced Pluripotent Stem Cells Delineates the Kinetics of Key Regulators of Hematopoiesis. Stem Cells 34, 1513-1526.
52. Trump, L. R., Nayak, R. C., Singh, A. K., Emberesh, S., Wellendorf, A. M., Lutzko, C. M., and Cancelas, J. A. (2019). Neutrophils Derived from Genetically Modified Human Induced Pluripotent Stem Cells Circulate and Phagocytose Bacteria In Vivo. Stem Cells Transl. Med. 8, 557-567.
53. Wang, D., Starr, R., Chang, W. C., Aguilar, B., Alizadeh, D., Wright, S. L., Yang, X., Brito, A., Sarkissian, A., Ostberg, J. R., et al. (2020a). Chlorotoxin-directed CAR T cells for specific and effective targeting of glioblastoma. Sci. Transl. Med. 12.
54. Wang, J., Toregrosa-Allen, S., Elzey, B. D., Utturkar, S., Lanman, N. A., Bernal-Crespo, V., Behymer, M. M., Knipp, G. T., Yun, Y., Veronesi, M. C., et al. (2020b). Tumor-responsive, multifunctional CAR-NK cells cooperate with impaired autophagy to infiltrate and target glioblastoma. BioRxiv 2020.10.07.330043.
55. Wang, T., Cao, L., Dong, X., Wu, F., De, W., Huang, L., and Wan, Q. (2020c). LINC01116 promotes tumor proliferation and neutrophil recruitment via DDX5-mediated regulation of IL-1β in glioma cell. Cell Death Dis. 11.
56. Xie, X., Shi, Q., Wu, P., Zhang, X., Kambara, H., Su, J., Yu, H., Park, S. Y., Guo, R., Ren, Q., et al. (2020). Single-cell transcriptome profiling reveals neutrophil heterogeneity in homeostasis and infection. Nat. Immunol. 21, 1119-1133.
57. Yan, J., Kloecker, G., Fleming, C., Bousamra, M., Hansen, R., Hu, X., Ding, C., Cai, Y., Xiang, D., Donninger, H., et al. (2014). Human polymorphonuclear neutrophils specifically recognize and kill cancerous cells. Oncoimmunology 3, e950163.
58. Zhao, Y., Rahmy, S., Liu, Z., Zhang, C., and Lu, X. (2020). Rational targeting of immunosuppressive neutrophils in cancer. Pharmacol. Ther. 212.
59. Zhu, H., Lai, Y.-S., Li, Y., Blum, R. H., and Kaufman, D. S. (2018). Concise Review: Human Pluripotent Stem Cells to Produce Cell-Based Cancer Immunotherapy. Stem Cells 36, 134-145.
60. Chang, Y., Hellwarth, P. B., Randolph, L. N., Sun, Y., Xing, Y., Zhu, W., Lian, X. L., and Bao, X. (2020). Fluorescent indicators for continuous and lineage-specific reporting of cell-cycle phases in human pluripotent stem cells. Biotechnol. Bioeng. bit.27352.
61. Kim, G. B., Aragon-Sanabria, V., Randolph, L., Jiang, H., Reynolds, J. A., Webb, B. S., Madhankumar, A., Lian, X., Connor, J. R., Yang, J., et al. (2020). High-affinity mutant Interleukin-13 targeted CAR T cells enhance delivery of clickable biodegradable fluorescent nanoparticles to glioblastoma. Bioact. Mater. 5, 624-635.
62. Ng, E. S., Azzola, L., Bruveris, F. F., Calvanese, V., Phipson, B., Vlahos, K., Hirst, C., Jokubaitis, V. J., Yu, Q. C., Maksimovic, J., et al. (2016). Differentiation of human embryonic stem cells to HOXA+ hemogenic vasculature that resembles the aorta-gonad-mesonephros. Nat. Biotechnol. 34, 1168-1179.

1. A stage-specific process for manufacturing a population of neutrophils from human pluripotent stem cells (hPSCs) comprising the steps of:
(a) preparing hPSCs;
(b) stimulating said hPSCs with a glycogen synthase kinase 3 β (GSK3β) inhibitor to produce a population of CD34+ hemogenic endothelium cells;
(c) stimulating said CD34+ hemogenic endothelium cells with a transforming growth factor β (TGFβ) inhibitor to produce a population of CD45+ hematopoietic cells; and
(d) stimulating said CD45+ hematopoietic cells with granulocyte macrophage colony-stimulating factor (GM-CSF) and a retinoic acid receptor agonist to afford a population of CD11b+/CD16+ neutrophils.

2. The stage-specific process of claim 1, wherein said hPSCs comprise human embryonic stem cells (hESCs) and induced pluripotent stem cells (iPSCs).

3. The stage-specific process of claim 1, wherein said GSK3β inhibitor is CHIR99021, CHIR98014, or similar chemicals.

4. The stage-specific process of claim 1, wherein said TGFβ inhibitor is SB431542, A83-01 or similar chemicals.

5. The stage-specific process of claim 1, wherein said a retinoic acid receptor agonist is AM80, AM50, or similar chemicals.

6. The stage-specific process of claim 1, wherein step b. is carried out in the presence of vascular endothelial growth factor (VEGF).

7. The stage-specific process of claim 1, wherein step c. is carried out in the presence of stem cell factor (SCF) and FMS-like tyrosine kinase 3 ligand (FLT3L).

8. The stage-specific process for manufacturing a population of neutrophils of claim 1, wherein preparing hPSCs comprises:
(i) knocking a CAR expression gene construct into the AAVS1 safe harbor locus of an adeno-associated virus S1 (AAVS1) plasmid in human pluripotent stem cells (hPSCs) via CRISPR/Cas9-mediated homologous recombination;
(ii) isolating successfully targeted single cell-derived hPSC colonies or hPSC cell mixture to afford a stable CAR-expressing hPSC cell line; and
(iii) preparing hPSCs from said stable CAR-expressing hPSC cell line; and
wherein the population of CD11b+/CD16+ neutrophils comprises
a population of CAR neutrophils.

9. The stage-specific process of claim 8, which further comprises the initial steps of:
(a′) preparing a CAR expression gene construct; and (a″) constructing a AAVS1 plasmid.

10. The stage-specific process of claim 8, wherein the CAR comprises chlorotoxin or IL13, or other extracellular signaling domains, the transmembrane domain of CD4, CD28, CD32a or NKG2D, co-stimulatory domain 2B4, and the intracellular domain of CD3ζ or FcγR domains.

11. The stage-specific process of claim 10, wherein the CAR has the amino acid sequence of SEQ ID NO: 2.

12. The stage-specific process of claim 8, wherein the CAR has the amino acid sequence of SEQ ID NO: 1 or 3.

13. (canceled)

14. The stage-specific process of claim 2, where said hESCs comprises H9, H1 or other human embryonic stem cells.

15. The stage-specific process of claim 2, where said iPSCs comprises 6-9-9, 19-9-11 or other induced pluripotent stem cells.

16-19. (canceled)

20. The stage-specific process of claim 8, wherein step c. is carried out in the presence of stem cell factor (SCF) and FMS-like tyrosine kinase 3 ligand (FLT3L).

21. A process for manufacturing a population of chimeric antigen receptor (CAR) neutrophils from human pluripotent stem cells (hPSCs) comprising the steps of:
(a) constructing a PiggyBac transposon plasmid comprising a CAR expression gene;
(b) delivering the PiggyBac plasmid into a human pluripotent stem cell via nucleofection/electroporation;
(c) isolating successfully targeted single cell-derived human pluripotent stem cell (hPSC) colonies or hPSC cell mixture for stable CAR-expressing hPSC lines; and
(d) producing CAR-expressing neutrophils according to the process of:
(i) preparing hPSCs;
(ii) stimulating said hPSCs with a glycogen synthase kinase 3 β (GSK3β) inhibitor to produce a population of CD34+ hemogenic endothelium cells;
(iii) stimulating said CD34+ hemogenic endothelium cells with a transforming growth factor β (TGFβ) inhibitor to produce a population of CD45+ hematopoietic cells; and
(iv) stimulating said CD45+ hematopoietic cells with granulocyte macrophage colony-stimulating factor (GM-CSF) and a retinoic acid receptor agonist to afford a population of CD11b+/CD16+ neutrophils.

22. The process of claim 21, wherein said hPSCs comprise human embryonic stem cells (hESCs) and induced pluripotent stem cells (iPSCs).

23. The process of claim 22, where said hESCs comprises H9, H1 or other human embryonic stem cells.

24. The process of claim 22, where said iPSCs comprises 6-9-9, 19-9-11 or other induced pluripotent stem cells.

25. The process of claim 21, wherein the CAR comprises chlorotoxin or IL13, or other extracellular signaling domains, the transmembrane domain of CD4, CD28, CD32a or NKG2D, co-stimulatory domain 2B4, and the intracellular domain of CD3ζ or FcγR domains.

26. The process of claim 25, wherein the CAR has the amino acid sequence of SEQ ID NO: 2.

27. The process of claim 21, wherein the CAR has the amino acid sequence of SEQ ID NO: 1 or 3.

28. An engineered neutrophil cell line from human pluripotent stem cells (hPSCs) comprising a chimeric antigen receptor (CAR) having the amino acid sequence of SEQ ID NO: 1.

29. An engineered neutrophil cell line from human pluripotent stem cells (hPSCs) comprising a chimeric antigen receptor (CAR) comprises chlorotoxin or IL13, or other extracellular signaling domains, the transmembrane domain of CD4, CD28, CD32a or NKG2D, co-stimulatory domain 2B4, and the intracellular domain of CD3ζ or FcγR domains.

30. The engineered neutrophil cell line of claim 29, wherein the CAR has the amino acid sequence of SEQ ID NO: 2.

31. An engineered neutrophil cell line from human pluripotent stem cells (hPSCs) comprising a chimeric antigen receptor (CAR) having the amino acid sequence of SEQ ID NO: 3.

32. A pharmaceutical composition comprising:
a population of isolated neutrophils obtained by performing a stage-specific process for manufacturing a population of neutrophils from human pluripotent stem cells (hPSCs) comprising the steps of:
(a) preparing hPSCs;
(b) stimulating said hPSCs with a glycogen synthase kinase 3 β (GSK3β) inhibitor to produce a population of CD34+ hemogenic endothelium cells;
(c) stimulating said CD34+ hemogenic endothelium cells with a transforming growth factor β (TGFβ) inhibitor to produce a population of CD45+ hematopoietic cells; and
(d) stimulating said CD45+ hematopoietic cells with granulocyte macrophage colony-stimulating factor (GM-CSF) and a retinoic acid receptor agonist to afford a population of CD11b+/CD16+ neutrophils; and a pharmaceutically acceptable carrier.

33. A method of treating cancer in a subject in need thereof, which method comprises administering to the subject a therapeutically effective amount of a population of the neutrophils obtained by performing a stage-specific process for manufacturing a population of neutrophils from human pluripotent stem cells (hPSCs) comprising the steps of:
(a) preparing hPSCs;
(b) stimulating said hPSCs with a glycogen synthase kinase 3 β (GSK3β) inhibitor to produce a population of CD34+ hemogenic endothelium cells;
(c) stimulating said CD34+ hemogenic endothelium cells with a transforming growth factor β (TGFβ) inhibitor to produce a population of CD45+ hematopoietic cells; and
(d) stimulating said CD45+ hematopoietic cells with granulocyte macrophage colony-stimulating factor (GM-CSF) and a retinoic acid receptor agonist to afford a population of CD11b+/CD16+ neutrophils and a pharmaceutically acceptable carrier, whereupon the subject is treated for cancer.

34. The method of claim 33, wherein the cancer is a brain tumor, prostate cancer or other cancers.

35. The method of claim 34, wherein the brain tumor is a glioma.

36. The method of claim 35, wherein the glioma is a glioblastoma.

37. The method of claim 33, wherein the cancer expresses the protein matrix metallopeptidase 2, IL-13 receptor, PSMA, or other cancer antigens.

38. The method of claim 33, wherein the population of neutrophils or the pharmaceutical composition comprising same is administered systemically or intracranially.

39-42. (canceled)
